(19) United States
(12) Patent Application Publication
(10) Pub. No.: US 2007/0015217 A1
(43) Pub. Date: Jan. 18, 2007

(54) Nucleic acid molecules, polypeptides and uses therefor, including diagnosis and treatment of Alzheimer's disease

(76) Inventors: L. Kathryn Durham, New London, CT (US); David L. Friedman, Madison, CT (US); Herath Mudiyanselage Athula Chandrasiri Herath, Abingdom (GB); Lida H. Kimmel, Chester, CT (US); Rajesh Bhikhu Parekh, New Wendlebury (GB); David M. Potter, Ledyard, CT (US); Christian Rohlff, Oxford (GB); B. Michael Silber, Madison, CT (US); Thomas R. Stiger, Pawcatuck, CT (US); P. Trey Sunderland, Chevy Chase, MD (US); Robert Reid Townsend, Oxford (GB); W. Frost White, Ledyard, CT (US); Stephen A. Williams, Groton, CT (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/078,000

(22) Filed: Mar. 11, 2005

Related U.S. Application Data
(62) Division of application No. 09/826,290, filed on Apr. 3, 2001, now abandoned.
(60) Provisional application No. 60/194,504, filed on Apr. 3, 2000.
(60) Provisional application No. 60/253,647, filed on Nov. 28, 2000.

(51) Int. Cl. G01N 33/567; G01N 33/53

(52) U.S. Cl. 435/7.2

(57) ABSTRACT

The present invention provides methods and compositions for screening, diagnosis and prognosis of Alzheimer's disease, for monitoring the effectiveness of Alzheimer's disease treatment, and for drug development. Alzheimer's Disease-Associated Features (AFs), detectable by two-dimensional electrophoresis of cerebrospinal fluid, serum or plasma are described. The invention further provides Alzheimer's Disease-Associated Protein Isoforms (APIs) detectable in cerebrospinal fluid, serum or plasma, preparations comprising isolated APIs, antibodies, pharmaceutical compositions, diagnostic and therapeutic methods, and kits comprising or based on the same.

### INTRODUCTION

The present invention relates to the identification of proteins and protein isoforms that are associated with predisposition to Alzheimer's Disease and its onset and development, and of genes and nucleic acid molecules, encoding the same, and to their use for e.g., clinical screening, diagnosis, treatment, as well as for drug screening and drug development.

### BACKGROUND OF THE INVENTION

Alzheimer's Disease (AD) is an increasingly prevalent form of neurodegeneration that accounts for approximately 50%-60% of the overall cases of dementia among people over 65 years of age. It currently affects an estimated 15 million people worldwide and owing to the relative increase of elderly people in the population its prevalence is likely to increase over the next 2 to 3 decades. Alzheimer's disease is a progressive disorder with a mean duration of around 8.5 years between onset of clinical symptoms and death. Death of pyramidal neurons and loss of neuronal synapses in brains regions associated with higher mental functions results in the typical symptomology, characterized by gross and progressive impairment of cognitive function (Francis et al., 1999, J. Neurol. Neurosurg. Psychiatry 66:137-47). Currently, a diagnosis of Alzheimer's. disease requires a careful medical history and physical examination; a detailed neurological and psychiatric examination; laboratory blood studies to exclude underlying metabolic and medical illnesses that masquerade as AD; a mental status assessment and formal cognitive tests; and a computed tomographic scan or magnetic resonance image of the brain (Growdon, J H., 1995, Advances in the diagnosis of Alzheimer's disease. In: Iqbal, K., Mortimer, J A., Winblad, B., Wisniewski, H M eds Research Advances in Alzheimer's Disease and Related Disorders. New York, N.Y.: John Wiley & Sons Inc. 1995:139-153). Due to the time consuming nature of these tests, their expense, and their inconvenience to patients, it would be highly desirable to measure a substance or substances in body samples, such as samples of cerebrospinal fluid (CSF), blood or urine, that would lead to a positive diagnosis of Alzheimer's disease or that would help to exclude AD from the differential diagnosis. Since the CSF bathes the brain, changes in its protein composition may most accurately reveal alterations in brain protein expression patterns that are causatively or diagnostically linked to the disease.

Current candidate biomarkers for Alzheimer's disease include: (1) mutations in presenilin 1 (PS1), presenilin 2 (PS2) and amyloid precursor protein (APP) genes; (2) the detection of alleles of apoplipoprotein E (ApoE); and (3) altered concentrations of amyloid β-peptides (AP), tau protein, and neuronal thread protein (NTP) in the CSF. See, e.g., Neurobiology of Aging 19:109-116 (1998) for a review. Mutations in PS1, PS2 and APP genes are indicative of early-onset familial Alzheimer's disease. However, early-onset familial Alzheimer's disease is relatively rare; only 120 families worldwide are currently known to carry deterministic mutations (Neurobiology of Aging 19:109-116 (1998)). The detection of the e4 allele of ApoE has been shown to correlate with late-onset and sporadic forms of Alzheimer's disease. However, e4 alone cannot be used as a biomarker for Alzheimer's disease since e4 has been detected in many individuals not suffering from Alzheimer's disease and the absence of e4 does not exclude Alzheimer's disease (Neurobiology of Aging 19:109-116 (1998)).

A decrease in the Aβ peptide Aβ42 and an increase in tau protein in the CSF of Alzheimer's disease have been shown to correlate with the presence of Alzheimer's disease (Neurobiology of Aging 19:109-116 (1998)). However, the specificity and sensitivity of Aβ42 and tau protein as biomarkers of Alzheimer's disease are modest. For example, it has been difficult to determine a cutoff level of CSF tau protein that is diagnostically informative. Also, elevated levels of NTP in the CSF of postmortem subjects has been shown to correlate with the presence of Alzheimer's disease (Neurobiology of Aging 19:109-116 (1998)). Therefore, a need exists to identify sensitive and specific biomarkers for the diagnosis of Alzheimer's disease in living subjects.

### SUMMARY OF THE INVENTION

The present invention provides methods and compositions for screening, diagnosis and treatment of Alzheimer's disease and for screening and development of drugs for treatment of Alzheimer's disease.

A first aspect of the invention provides methods for identification of Alzheimer's disease that comprise analyzing a sample of CSF by two-dimensional electrophoresis to detect the presence or level of at least one Alzheimer's Disease-Associated Feature (AF), e.g., one or more of the AFs disclosed herein, or any combination thereof. These methods are also suitable for clinical screening, prognosis, monitoring the results of therapy, for identifying patients most likely to respond to a particular therapeutic treatment, drug screening and development, and identification of new targets for drug treatment.

A second aspect of the invention provides methods for diagnosis of Alzheimer's disease that comprise detecting in a sample of CSF the presence or level of at least one Alzheimer's Disease-Associated Protein Isoform (API), e.g., one or more of the APIs disclosed herein or any combination thereof.

A third aspect of the invention provides antibodies, e.g., monoclonal and polyclonal and chimeric (bispecific) antibodies capable of immunospecific binding to an API, e.g., an API disclosed herein.

A fourth aspect of the invention provides a preparation comprising an isolated API, i.e., an API substantially free from proteins or protein isoforms having a significantly different isoelectric point or a significantly different apparent molecular weight from the API.

A fifth aspect of the invention provides kits that may be used in the above recited methods and that may comprise single or multiple preparations, or antibodies, together with other reagents, labels, substrates, if needed, and directions for use. The kits may be used for diagnosis of disease, or may be assays for the identification of new diagnostic and/or therapeutic agents.

A sixth aspect of the invention provides methods of treating Alzheimer's disease, comprising administering to a subject a therapeutically effective amount of an agent that modulates (e.g., upregulates or downregulates) the expression or activity (e.g. enzymatic or binding activity), or both, of an API in subjects having Alzheimer's disease.

A seventh aspect of the invention provides methods of screening for agents that modulate (e.g., upregulate or downregulate) a characteristic of, e.g., the expression or the enzymatic or binding activity, of an API, an API analog, or an API-related polypeptide.

Other objects and advantages will become apparent from a review of the ensuing detailed description taken in conjunction with the following illustrative drawing.

### BRIEF DESCRIPTION OF THE FIGURE

FIG. 1 is an image obtained from 2-dimensional electrophoresis of normal CSF, which has been annotated to identify twelve landmark features, designated CSF1 to CSF12, and which are illustrative of an embodiment of an aspect of the present invention.

### DETAILED DESCRIPTION OF THE INVENTION

The present invention described in detail below provides methods, compositions and kits useful, e.g., for screening, diagnosis and treatment of Alzheimer's disease in a mammalian subject, and for drug screening and drug development. The invention also encompasses the administration of therapeutic compositions to a mammalian subject to treat or prevent Alzheimer's disease. The mammalian subject may be a non-human mammal, but is preferably human, more preferably a human adult, i.e. a human subject at least 21 (more particularly at least 35, at least 50, at least 60, at least 70, or at least 80) years old. For clarity of disclosure, and not by way of limitation, the invention will be described with respect to the analysis of CSF samples. However, as one skilled in the art will appreciate, based on the present description the assays and techniques described below can be applied to other types of samples, including a body fluid (e.g. blood, serum, plasma or saliva), a tissue sample from a subject at risk of having or developing Alzheimer's disease (e.g. a biopsy such as a brain biopsy) or homogenate thereof. The methods and compositions of the present invention are useful, such as for example, screening, diagnosis and treatment of a living subject, but may also be used for postmortem diagnosis in a subject, for example, to identify family members of the subject who are at risk of developing the same disease.

The following definitions are provided to assist in the review of the instant disclosure.

### Definitions

“Diagnosis” refers to diagnosis, prognosis, monitoring, selecting participants in clinical trials, and identifying patients most likely to respond to a particular therapeutic treatment. “Treatment” refers to therapy, prevention and prophylaxis.

“Agent” refers to all materials that may be used to prepare pharmaceutical and diagnostic compositions, or that may be compounds, nucleic acids, polypeptides, fragments, isoforms, or other materials that may be used independently for such purposes, all in accordance with the present invention.

“API analog” refers to a polypeptide that possesses similar or identical function(s) as an API but need not necessarily comprise an amino acid sequence that is similar or identical to the amino acid sequence of the API, or possess a structure that is similar or identical to that of the API. As used herein, an amino acid sequence of a polypeptide is “similar” to that of an API if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the API; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the API; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the API. As used herein, a polypeptide with “similar structure” to that of an API refers to a polypeptide that has a similar secondary, tertiary or quartemary structure as that of the API. The structure of a polypeptide can determined by methods known to those skilled in the art, including but not limited to, X-ray crystallography, nuclear magnetic resonance, and crystallographic electron microscopy.

“API fusion protein” refers to a polypeptide that comprises (i) an amino acid sequence of an API, an API fragment, an API-related polypeptide or a fragment of an API-related polypeptide and (ii) an amino acid sequence of a heterologous polypeptide (i.e., a non-API, non-API fragment or non-API-related polypeptide). “API homolog” refers to a polypeptide that comprises an amino acid sequence similar to that of an API but does not necessarily possess a similar or identical function as the API.

“API ortholog” refers to a non-human polypeptide that (i) comprises an amino acid sequence similar to that of an API and (ii) possesses a similar or identical function to that of the API.

“API-related polypeptide” refers to an API homolog, an API analog, an isoform of API, an API ortholog, or any combination thereof.

“Chimeric Antibody” refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816397, which are incorporated herein by reference in their entirety.)

“Derivative” refers to a polypeptide that comprises an amino acid sequence of a second polypeptide which has been altered by the introduction of amino acid residue substitutions, deletions or additions. The derivative polypeptide possess a similar or identical function as the second polypeptide.

“Fragment” refers to a peptide or polypeptide comprising an amino acid sequence of at least 5 amino acid residues (preferably, at least. 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. The fragment of an API may or may not possess a functional activity of the a second polypeptide.

“Fold change” includes “fold increase” and “fold decrease” and refers to the relative increase or decrease in abundance of an AF or the relative increase or decrease in expression or activity of a polypeptide (e.g. an API) in a first sample or sample set compared to a second sample (or sample set). An AF or polypeptide fold change may be measured by any technique known to those of skill in the art, albeit the observed increase or decrease will vary depending upon the technique used. Preferably, fold change is determined herein as described in the Examples infra.

“Isoform” refers to variants of a polypeptide that are encoded by the same gene, but that differ in their pI or MW, or both. Such isoforms can differ in their amino acid composition (e.g. as a result of alternative mRNA or premRNA processing, e.g. alternative splicing or limited proteolysis) and in addition, or in the alternative, may arise from differential post-translational modification (e.g., glycosylation, acylation, phosphorylation).

“Modulate” in reference to expression or activity of an API or an API-related polypeptide refers to any change, e.g., upregulation or downregulation, of the expression or activity of the API or an API-related polypeptide. Those skilled in the art, based on the present disclosure, will understand that such modulation can be determined by assays known to those of skill in the art.

“Treatment” refers to the administration of medicine or the performance of medical procedures with respect to a patent, for either prophylaxis (prevention) or to cure the infirmity or malady in the instance where the patient is afflicted.

The percent identity of two amino acid sequences or of two nucleic acid sequences can be or is generally determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti (1994) Comput. Appl. Biosci., 10 :3-5; and FASTA described in Pearson and Lipman (1988) Proc. Natl. Acad. Sci. 85:2444-8. Within FASTA, ktup is a control option that sets the sensitivity and speed of the search.

Cerebrospinal fluid (CSF) refers to the fluid that surrounds the bulk of the central nervous system, as described in Physiological Basis of Medical Practice (J. B. West, ed., Williams and Wilkins, Baltimore, Md. 1985). CSF includes ventricular CSF and lumbar CSF.

Alzheimer's Disease-Associated Features (AFs)

In one aspect of the invention, two-dimensional electrophoresis is used to analyze CSF from a subject, preferably a living subject, in order to detect or quantify the expression of one or more Alzheimer's Disease-Associated Features (AFs) for screening, treatment or diagnosis of Alzheimer's disease. As used herein, “two-dimensional electrophoresis” (2D-electrophoresis) means a technique comprising isoelectric focusing, followed by denaturing electrophoresis; this generates a two-dimensional gel (2D-gel) containing a plurality of separated proteins. Preferably, the step of denaturing electrophoresis uses polyacrylamide electrophoresis in the presence of sodium dodecyl sulfate (SDS-PAGE). Especially preferred are the highly accurate and automatable methods and apparatus (“the Preferred Technology”) described in International Application No. 97GB3307 (published as WO 98/23950) and in U.S. Application No. 08/980,574, both filed Dec. 1, 1997, each of which is incorporated herein by reference in its: entirety with particular reference to the protocol at pages 23-35. Briefly, the Preferred Technology provides efficient, computer-assisted methods and apparatus for identifying, selecting and characterizing biomolecules (e.g. proteins, including glycoproteins) in a biological sample. A two-dimensional array is generated by separating biomolecules on a two-dimensional gel according to their electrophoretic mobility and isoelectric point. A computer-generated digital profile of the array is generated, representing the identity, apparent molecular weight, isoelectric point, and relative abundance of a plurality of biomolecules detected in the two-dimensional array, thereby permitting computer-mediated comparison of profiles from multiple biological samples, as well as computer aided excision of separated proteins of interest.

A particular scanner for detecting fluorescently labeled proteins is described in WO 96/36882 and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686, the contents of each of which are incorporated herein by reference. These documents describe an image scanner designed specifically for automated, integrated operation at high speeds. The scanner can image gels that have been stained with fluorescent dyes or silver stains, as well as storage phosphor screens. The Basiji thesis provides a phase-sensitive detection system for discriminating modulated fluorescence from baseline noise due to laser scatter or homogeneous fluorescence, but the scanner can also be operated in a non-phase-sensitive mode. This phase-sensitive detection capability would increase the sensitivity of the instrument by an order of magnitude or more compared to conventional fluorescence imaging systems. The increased sensitivity would reduce the sample-preparation load on the upstream instruments while the enhanced image quality simplifies image analysis downstream in the process.

A more highly preferred scanner is the Apollo 2 scanner (Oxford Glycosciences, Oxford, UK), which is a modified version of the above described scanner. In the Apollo 2 scanner, the gel is transported through the scanner on a precision lead-screw drive system. This is preferable to laying the glass plate on the belt-driven system that is described in the Basiji thesis, as it provides a reproducible means of accurately transporting the gel past the imaging optics.

In the Apollo 2 scanner, the gel is secured against three alignment stops that rigidly hold the glass plate in a known position. By doing this in conjunction with the above precision transport system, the absolute position of the gel can be predicted and recorded. This ensures that co-ordinates of each feature on the gel can be determined more accurately and communicated, if desired, to a cutting robot for excision of the feature. In the Apollo 2 scanner, the carrier that holds the gel has four integral fluorescent markers for use to correct the image geometry. These markers are a quality control feature that confirms that the scanning has been performed correctly.

In comparison to the scanner described in the Basiji thesis, the optical components of the Apollo 2 scanner have been inverted. In the Apollo 2 scanner, the laser, mirror, waveguide and other optical components are above the glass plate being scanned. The scanner described in the Basiji thesis has these components underneath. In the Apollo 2 scanner, the glass plate is mounted onto the scanner gel side down, so that the optical path remains through the glass plate. By doing this, any particles of gel that may break away from the glass plate will fall onto the base of the instrument rather than into the optics. This does not affect the functionality of the system, but increases its reliability.

Still more preferred is the Apollo 3 scanner, in which the signal output is digitized to the full 16-bit data without any peak saturation or without square root encoding of the signal. A compensation algorithm has also been applied to correct for any variation in detection sensitivity along the path of the scanning beam. This variation is due to anomalies in the optics and differences in collection efficiency across the waveguide. A calibration is performed using a perspex plate with an even fluorescence throughout. The data received from a scan of this plate are used to determine the multiplication factors needed to increase the signal from each pixel level to a target level. These factors are then used in subsequent scans of gels to remove any internal optical variations.

“Feature” refers to a spot detected in a 2D gel, and the term “Alzheimer's Disease-Associated Features” (AF) refers to a feature that is differentially present in a sample (e.g. a sample of CSF) from a subject having Alzheimer's disease compared with a sample (e.g. -a sample of CSF) from a subject free from Alzheimer's disease. As used herein, a feature (or a protein isoform of API, as defined infra) is “differentially present” in a first sample with respect to a second sample when a method for detecting the feature, isoform or API (e.g., 2D electrophoresis or an immunoassay) gives a different signal when applied to the first and second samples. A feature, isoform or API is “increased” in the first sample with respect to the second if the method of detection indicates that the feature, isoform or API is more abundant in the first sample than in the second sample, or if the feature, isoform or API is detectable in the first sample and substantially undetectable in the second sample. Conversely, a feature, isoform or API is “decreased” in the first sample with respect to the second if the method of detection indicates that the feature, isoform or API is less abundant in the first sample than in the second sample or if the feature, isoform or API is undetectable in the first sample and detectable in the second sample.

Particularly, the relative abundance of a feature in two samples is determined in reference to its normalized signal, in two steps. First, the signal obtained upon detecting the feature in a sample is normalized by reference to a suitable background parameter, e.g., (a) to the total protein in the sample being analyzed (e.g., total protein loaded onto a gel); (b) to an Expression Reference Feature (ERF) i.e., a feature whose abundance is substantially invariant, within the limits of variability of the Preferred Technology, in the population of subjects being examined, e.g. the ERFs disclosed below, or (c) more preferably to the total signal detected as the sum of each of all proteins in the sample.

Secondly, the normalized signal for the feature in one sample or sample set is compared with the normalized signal for the same feature in another sample or sample set in order to identify features that are “differentially present” in the first sample (or sample set) with respect to the second.

In accordance with an aspect of the present invention, the AFs disclosed herein have been identified by comparing CSF samples from subjects having Alzheimer's disease against CSF samples from subjects free from Alzheimer's disease. Subjects free from Alzheimer's disease include subjects with no known disease or condition (normal subjects) and subjects with diseases (including neurological and neurodegenerative diseases) other than Alzheimer's disease.

Two groups of AFs have been identified through the methods and apparatus of the Preferred Technology. The first group consists of AFs that are decreased in the CSF of subjects having Alzheimer's disease as compared with the CSF of subjects free from Alzheimer's disease. These AFs can be described by apparent molecular weight (MW) and isoelectric point (pI) as provided in Table I.

TABLE I
AFs Decreased in CSF of Subjects Having Alzheimer's Disease
(a) Data from Mastergroup Analysis

| AF# | Fold Decrease^{#} | pI | MW (Da) | p value* |
|---|---|---|---|---|
| AF-1 | 1.41 | 4.79 | 150081 | 0.001695⁽¹⁾ |
| AF-2 | 1.34 | 4.28 | 21349 | 0.000133⁽¹⁾ |
| AF-3 | 1.47 | 8.10 | 34846 | 0.000083⁽¹⁾ |
| AF-4 | 1.56 | 4.38 | 21160 | 0.001192⁽¹⁾ |
| AF-5 | 1.51 | 7.34 | 36554 | 0.000010⁽¹⁾ |
| AF-6 | 1.46 | 4.91 | 29812 | 0.000003⁽¹⁾ |
| AF-7 | 1.24 | 4.25 | 20787 | 0.003558⁽¹⁾ |
| AF-8 | 1.44 | 4.93 | 187927 | 0.002221⁽¹⁾ |
| AF-9 | 1.34 | 5.21 | 136768 | 0.000799⁽¹⁾ |
| AF-10 | 1.30 | 5.19 | 17694 | 0.000400⁽¹⁾ |
| AF-13 | 1.37 | 6.01 | 184530 | 0.000100⁽¹⁾ |
| AF-14 | 1.52 | 4.72 | 63166 | 0.009387⁽¹⁾ |
| AF-15 | 1.38 | 4.47 | 38970 | 0.000437⁽¹⁾ |
| AF-16 | 1.22 | 5.19 | 46876 | 0.000697⁽¹⁾ |
| AF-17 | 1.38 | 5.82 | 50294 | 0.000126⁽¹⁾ |
| AF-18 | 1.30 | 4.87 | 49219 | 0.020661⁽¹⁾ |
| AF-19 | 1.24 | 4.82 | 12454 | 0.00146⁽¹⁾ |
| AF-20 | 1.30 | 4.43 | 16818 | 0.000322⁽¹⁾ |
| AF-21 | 1.30 | 5.40 | 141094 | 0.000560⁽¹⁾ |
| AF-22 | 1.36 | 4.93 | 133773 | 0.011000⁽¹⁾ |
| AF-23 | 1.21 | 4.50 | 32473 | 0.000209⁽⁴⁾ |
| AF-24 | 1.20 | 5.31 | 46663 | 0.000871⁽¹⁾ |
| AF-25 | 1.19 | 5.68 | 36700 | 0.00251⁽¹⁾ |
| AF-26 | 1.31 | 8.11 | 32305 | 0.002204⁽¹⁾ |
| AF-27 | 1.26 | 5.33 | 141371 | 0.010447⁽¹⁾ |
| AF-28 | 1.06 | 5.13 | 158568 | 0.000100⁽¹⁾ |
| AF-29 | 1.27 | 9.22 | 47059 | 0.000028⁽¹⁾ |
| AF-30 | 1.13 | 5.67 | 48057 | 0.000100⁽¹⁾ |
| AF-31 | 1.15 | 6.07 | 91258 | 0.012712⁽⁴⁾ |
| AF-32 | 1.15 | 6.17 | 48958 | 0.008321⁽⁴⁾ |
| AF-33 | 1.06 | 4.41 | 42104 | 0.000126⁽¹⁾ |
| AF-34 | 1.14 | 4.54 | 145408 | 0.017268⁽⁴⁾ |
| AF-35 | 1.22 | 5.21 | 18623 | 0.001094⁽¹⁾ |
| AF-36 | 1.17 | 5.78 | 14416 | 0.010744⁽¹⁾ |
| AF-37 | 1.29 | 6.91 | 33523 | 0.000087⁽¹⁾ |
| AF-38 | 1.18 | 6.47 | 29535 | 0.002759⁽¹⁾ |
| AF-39 | 1.30 | 7.50 | 35510 | 0.002858⁽¹⁾ |
| AF-40 | 1.22 | 7.29 | 38617 | 0.001187⁽¹⁾ |
| AF-41 | 1.11 | 5.85 | 17345 | 0.016690⁽⁴⁾ |
| AF-42 | 1.10 | 5.04 | 18662 | 0.002252⁽¹⁾ |
| AF-43 | 1.13 | 9.83 | 14065 | 0.003303⁽¹⁾ |
| AF-44 | 1.10 | 6.63 | 102328 | 0.020753⁽⁴⁾ |
| AF-45 | 1.09 | 6.04 | 46998 | 0.031910⁽⁴⁾ |
| AF-46 | 1.09 | 4.71 | 19802 | 0.008437⁽⁴⁾ |
| AF-47 | 1.09 | 5.99 | 49664 | 0.002187⁽¹⁾ |
| AF-48 | 1.32 | 5.32 | 122332 | 0.006582⁽¹⁾ |
| AF-49 | 1.07 | 6.94 | 27576 | 0.010068⁽⁴⁾ |
| AF-50 | 1.07 | 6.82 | 71337 | 0.035409⁽⁴⁾ |
| AF-51 | 1.04 | 5.70 | 34388 | 0.006156⁽⁴⁾ |
| AF-76 | 1.11 | 5.59 | 45537 | 0.001973⁽¹⁾ |
| AF-149 | 1.15 | 4.82 | 190721 | 0.003541⁽¹⁾ |
| AF-150 | 1.24 | 6.87 | 157592 | 0.000100⁽¹⁾ |
| AF-152 | 1.09 | 5.04 | 81703 | 0.002800⁽¹⁾ |
| AF-154 | 1.06 | 5.03 | 67307 | 0.000100⁽¹⁾ |
| AF-155 | 1.38 | 9.21 | 64021 | 0.000070⁽¹⁾ |
| AF-156 | 9.75 | 4.36 | 58083 | 0.001568⁽¹⁾ |
| AF-159 | 1.18 | 5.08 | 52008 | 0.000100⁽¹⁾ |
| AF-160 | 1.06 | 5.76 | 45729 | 0.001423⁽¹⁾ |
| AF-162 | 1.23 | 5.47 | 38663 | 0.001073⁽¹⁾ |
| AF-163 | 1.39 | 4.45 | 34879 | 0.001228⁽¹⁾ |
| AF-164 | 1.51 | 5.00 | 33485 | 0.01179⁽¹⁾ |
| AF-169 | 1.00 | 8.00 | 34362 | 0.000004⁽¹⁾ |
| AF-170 | 1.38 | 5.41 | 31886 | 0.005600⁽¹⁾ |
| AF-172 | 1.53 | 6.71 | 28747 | 0.01051⁽¹⁾ |
| AF-173 | 10.91 | 7.67 | 27476 | 0.003738⁽¹⁾ |
| AF-174 | 1.03 | 4.67 | 27811 | 0.002423⁽¹⁾ |
| AF-175 | 1.03 | 5.33 | 24936 | 0.044270⁽¹⁾ |
| AF-176 | 1.15 | 4.86 | 22248 | 0.012144⁽¹⁾ |
| AF-177 | 1.14 | 4.63 | 21103 | 0.006564⁽¹⁾ |
| AF-178 | 1.08 | 6.03 | 22247 | 0.05097⁽¹⁾ |
| AF-181 | 1.21 | 5.72 | 16336 | 0.004745⁽¹⁾ |
| AF-183 | 1.44 | 10.36 | 11160 | 0.000270⁽¹⁾ |
| AF-184 | 1.08 | 5.31 | 48769 | 0.004689⁽¹⁾ |
| AF-186 | 2.76 | 4.71 | 29693 | 0.002446⁽¹⁾ |
| AF-187 | 2.09 | 4.93 | 154156 | 0.000750⁽¹⁾ |
| AF-188 | 1.35 | 5.52 | 39355 | 0.007175⁽¹⁾ |
| AF-189 | 1.29 | 6.79 | 30719 | 0.000377⁽¹⁾ |
| AF-190 | 1.26 | 5.29 | 29663 | 0.000178⁽¹⁾ |
| AF-191 | 1.12 | 5.31 | 46663 | 0.000654⁽¹⁾ |

*The statistical technique used to calculate a given p value is indicated by
a footnote for each p value. The statistical techniques used for these group
analyzes were
⁽¹⁾a linear model, controlling for age and gender;
⁽²⁾classification trees;
⁽³⁾a logistic regression model and
⁽⁴⁾longitudinal analysis.
#Fold changes reported here are those calculated before adjustment for age
and gender.

(b) Data from Pooled Gel Analysis

| AF# | Fold Decrease^{#} | pI | MW (Da) |
|---|---|---|---|
| AF-78 | 2.80 | 5.59 | 158937 |
| AF-79 | 2.62 | 5.52 | 142378 |
| AF-80 | 2.78 | 5.56 | 142378 |
| AF-81 | 2.60 | 5.43 | 78299 |
| AF-82 | 7.25 | 6.69 | 74838 |
| AF-83 | 4.07 | 6.81 | 71920 |
| AF-84 | 4.44 | 6.94 | 73402 |
| AF-85 | 16.77 | 7.10 | 73878 |
| AF-86 | 4.86 | 5.24 | 67676 |
| AF-87 | 4.17 | 5.95 | 64179 |
| AF-88 | 6.48 | 5.36 | 66979 |
| AF-89 | 2.64 | 5.39 | 65155 |
| AF-90 | 4.81 | 7.61 | 62945 |
| AF-91 | 3.10 | 8.16 | 56352 |
| AF-92 | 2.63 | 6.18 | 50860 |
| AF-93 | 6.33 | 6.28 | 49268 |
| AF-94 | 11.17 | 4.38 | 45882 |
| AF-95 | 9.28 | 5.60 | 46036 |
| AF-96 | 11.07 | 4.43 | 44966 |
| AF-98 | 5.51 | 6.72 | 44664 |
| AF-99 | 10.1 | 5.92 | 44365 |
| AF-100 | 15.39 | 6.08 | 44068 |
| AF-101 | 9.82 | 4.47 | 44216 |
| AF-102 | 3.14 | 6.02 | 44216 |
| AF-103 | 3.57 | 5.93 | 42722 |
| AF-104 | 22.78 | 5.09 | 42184 |
| AF-105 | 4.11 | 5.19 | 42184 |
| AF-107 | 13.3 | 7.26 | 33226 |
| AF-108 | 19.0 | 7.54 | 33136 |
| AF-110 | 9.21 | 5.39 | 28237 |
| AF-111 | 14.85 | 5.68 | 27835 |
| AF-112 | 2.60 | 6.00 | 20681 |
| AF-114 | 8.98 | 6.80 | 18741 |
| AF-115 | 2.85 | 6.04 | 17422 |
| AF-116 | 6.85 | 6.68 | 14031 |
| AF-117 | 2.90 | 4.65 | 13983 |
| AF-118 | 15.19 | 6.94 | 11739 |
| AF-119 | 16.27 | 7.23 | 11699 |

#These features were identified as having a differential presence that is
significant on the basis of having at least a 2-fold difference in mean
intensity (i.e. a fold change threshold of at least 2) between Alzheimer's
CSF and normal CSF.

The second group consists of AFs that are increased in the CSF of subjects having Alzheimer's disease as compared with the CSF of subjects free from Alzheimer's disease. These AFs can be described by apparent molecular weight (MW) and isoelectric point (pI) as provided in Table II.

TABLE II
AFs Increased in CSF of Subjects Having Alzheimer's Disease
(a) Data from Mastergroup Analysis

| AF# | Fold Increase# | pI | MW (Da) | p value* |
|---|---|---|---|---|
| AF-52 | 2.81 | 6.30 | 32573 | 0.000009⁽⁴⁾ |
| AF-53 | 1.80 | 5.84 | 45302 | 0.016106⁽⁴⁾ |
| AF-54 | 1.76 | 5.12 | 17520 | 0.003235⁽¹⁾ |
| AF-55 | 1.29 | 8.10 | 12361 | 0.000482⁽¹⁾ |
| AF-56 | 1.49 | 8.56 | 52128 | 0.005771⁽¹⁾ |
| AF-57 | 1.46 | 6.30 | 68549 | 0.000274⁽¹⁾ |
| AF-58 | 1.40 | 5.01 | 14507 | 0.01182⁽¹⁾ |
| AF-59 | 1.37 | 6.74 | 33401 | 0.001351⁽¹⁾ |
| AF-60 | 1.38 | 5.39 | 33873 | 0.009818⁽¹⁾ |
| AF-61 | 1.34 | 6.76 | 54345 | Bag Tree 4 |
| | | | | Analysis⁽²⁾ |
| AF-62 | 1.31 | 6.60 | 31004 | 0.000027⁽¹⁾ |
| AF-63 | 1.24 | 5.97 | 14897 | 0.10696⁽¹⁾ |
| AF-64 | 1.20 | 6.67 | 68119 | 0.000731⁽¹⁾ |
| AF-65 | 1.22 | 7.19 | 58620 | 0.005833⁽⁴⁾ |
| AF-66 | 1.09 | 10.05 | 30092 | 0.000100⁽¹⁾ |
| AF-67 | 1.21 | 5.02 | 13735 | 0.006391⁽⁴⁾ |
| AF-68 | 1.21 | 9.06 | 35351 | 0.003575⁽¹⁾ |
| AF-69 | 1.19 | 5.01 | 46760 | 0.005125⁽⁴⁾ |
| AF-70 | 1.19 | 8.91 | 38789 | 0.021552⁽⁴⁾ |
| AF-71 | 1.20 | 6.44 | 68579 | 0.003848⁽¹⁾ |
| AF-72 | 1.15 | 5.00 | 43788 | 0.014917⁽⁴⁾ |
| AF-73 | 1.19 | 5.21 | 31615 | 0.000008⁽¹⁾ |
| AF-74 | 1.14 | 6.19 | 51934 | 0.054917⁽¹⁾ |
| AF-75 | 1.12 | 5.03 | 33671 | 0.002399⁽¹⁾ |
| AF-77 | 1.09 | 6.41 | 32196 | 0.035148⁽⁴⁾ |
| AF-151 | 1.13 | 5.28 | 137531 | Bag Tree 1 |
| | | | | Analysis⁽²⁾ |
| AF-153 | 1.23 | 9.85 | 69630 | 0.004906⁽¹⁾ |
| AF-157 | 1.70 | 4.99 | 55449 | 0.006272⁽¹⁾ |
| AF-161 | 1.00 | 5.18 | 44404 | 0.000600⁽¹⁾ |
| AF-165 | 1.88 | 7.17 | 34230 | 0.000035⁽¹⁾ |
| AF-166 | 1.20 | 8.54 | 33657 | 0.000009⁽¹⁾ |
| AF-167 | 1.31 | 5.69 | 33621 | 0.005400⁽¹⁾ |
| AF-168 | 1.00 | 7.66 | 33920 | 0.000013⁽¹⁾ |
| AF-171 | 1.10 | 4.98 | 29658 | 0.004242⁽¹⁾ |
| AF-179 | 1.64 | 5.26 | 20115 | Stepwise |
| | | | | Analysis⁽¹⁾ |
| AF-180 | 1.62 | 6.17 | 16255 | 0.005047⁽¹⁾ |
| AF-182 | 1.37 | 4.89 | 13651 | 0.005380⁽¹⁾ |
| AF-185 | 6.00 | 5.32 | 40323 | 0.005520⁽¹⁾ |
| AF-192 | 1.04 | 5.38 | 62756 | 0.000213⁽¹⁾ |

*The statistical technique used to calculate a given p value is indicated by
a footnote for each p value. The statistical techniques used for these group
analyzes were
⁽¹⁾a linear model, controlling for age and gender;
⁽²⁾classification trees;
⁽³⁾a logistic regression model and
⁽⁴⁾longitudinal analysis.
#Fold changes reported here are those calculated before adjustment for age
and gender.

(b) Data from Pooled Gel Analysis

| AF# | Fold Increase# | pI | MW (Da) |
|---|---|---|---|
| AF-121 | 11.7 | 5.42 | 105108 |
| AF-122 | 2.20 | 5.27 | 71060 |
| AF-123 | 6.35 | 7.31 | 64933 |
| AF-124 | 6.86 | 7.47 | 64736 |
| AF-125 | 2.34 | 4.77 | 61297 |
| AF-126 | 48.84 | 4.11 | 60374 |
| AF-127 | 6.79 | 4.98 | 59649 |
| AF-128 | 2.36 | 6.60 | 57865 |
| AF-129 | 2.90 | 5.29 | 54625 |
| AF-130 | 2.94 | 5.08 | 51880 |
| AF-131 | 5.19 | 6.54 | 50944 |
| AF-132 | 3.41 | 4.72 | 47414 |
| AF-133 | 3.08 | 5.12 | 44068 |
| AF-134 | 2.34 | 5.00 | 43516 |
| AF-137 | 4.41 | 4.98 | 36855 |
| AF-139 | 4.11 | 5.00 | 34295 |
| AF-140 | 110.32 | 6.80 | 32080 |
| AF-141 | 3.58 | 7.50 | 28440 |
| AF-142 | 2.66 | 6.75 | 27279 |
| AF-143 | 5.68 | 7.44 | 26066 |
| AF-144 | 2.71 | 6.56 | 20744 |
| AF-145 | 4.43 | 4.76 | 18069 |
| AF-146 | 2.18 | 4.94 | 12790 |
| AF-147 | 2.29 | 4.81 | 12790 |
| AF-148 | 5.04 | 5.32 | 11382 |

#These features were identified as having a differential presence that is
significant on the basis of having at least a 2-fold difference in mean
intensity (i.e. a fold change threshold of at least 2) between Alzheimer's
CSF and normal CSF.

For any given AF, the signal obtained upon analyzing CSF from subjects having Alzheimer's disease relative to the signal obtained upon analyzing CSF from subjects free from Alzheimer's disease will depend upon the particular analytical protocol and detection technique that is used. Accordingly, those skilled in the art will understand that any laboratory, based on the present description, can establish a suitable reference range for any AF in subjects free from Alzheimer's disease according to the analytical protocol and detection technique in use. In particular, at least one positive control CSF sample from a subject known to have Alzheimer's disease or at least one negative control CSF sample from a subject known to be free from Alzheimer's disease (and more preferably both positive and negative control samples) are included in each batch of test samples analyzed. In one embodiment, the level of expression of a feature is determined relative to a background value, which is defined as the level of signal obtained from a proximal region of the image that (a) is equivalent in area to the particular feature in question; and (b) contains no substantial discernable protein feature.

In a preferred embodiment, the signal associated with an AF in the CSF of a subject (e.g., a subject suspected of having or known to have Alzheimer's disease) is normalized with reference to one or more Expression Reference Features (ERFs) detected in the same 2D gel. As will be apparent to one of ordinary skill in the art, such ERFs may readily be determined by comparing different samples using techniques and protocols such as the Preferred Technology. Suitable ERFs include (but are not limited to) that described in the following table.

TABLE III
Expression Reference Features

| ERF# | pI | MW (Da) |
|---|---|---|
| ERF-1 | 5.94 | 18860 |
| ERF-2 | 6.04 | 47450 |

As those of skill in the art will readily appreciate, the measured MW and pI of a given feature or protein isoform will vary to some extent depending on the precise protocol used for each step of the 2D electrophoresis and for landmark matching. As used herein, the terms “MW” and “pI” are defined, respectively, to mean the apparent molecular weight in Daltons and the apparent isoelectric point of a feature or protein isoform as measured in careful accordance with the Reference Protocol identified in Section 6 below. When the Reference Protocol is followed and when samples are run in duplicate or a higher number of replicates, variation in the measured mean pI of an AF or API is typically less than 3% and variation in the measured mean MW of an AF or API is typically less than 5%. Where the skilled artisan wishes to diverge from the Reference Protocol, calibration experiments should be performed to compare the MW and pI for each AF or protein isoform as detected (a) by the Reference Protocol and (b) by the divergent.

The AFs of the invention can be used, for example, for detection, treatment, diagnosis, or the drug development or pharmaceutical products. In one embodiment of the invention, CSF from a subject (e.g., a subject suspected of having Alzheimer's disease) is analyzed by 2D electrophoresis for quantitative detection of one or more of the following AFs: AF-1, AF-2, AF-3, AF-4, AF-5, AF-6, AF-7, AF-8, AF-9, AF-10, AF-13, AF-14, AF-15, AF-16, AF-17, AF-18, AF-19, AF-20, AF-21, AF-22, AF-23, AF-24, AF-25, AF-26, AF-27, AF-28, AF-29, AF-30, AF-3 1, AF-32, AF-33, AF-34, AF-35, AF-36, AF-37, AF-38, AF-39, AF-40, AF-41, AF-42, AF-43, AF-44,AF-45, AF-46, AF-47, AF-48, AF-49, AF-50, AF-51, AF-76, AF-78, AF-79, AF-80, AF-81, AF-82, AF-83, AF-84, AF-85, AF-86, AF-87, AF-88, AF-89, AF-90, AF-91, AF-92, AF-93, AF-94, AF-95, AF-96, AF-98, AF-99, AF-100, AF-101, AF-102, AF-103, AF-104, AF-105, AF-107, AF-108, AF-110, AF-111, AF-112, AF-114, AF-115, AF-116, AF-117, AF-118, AF-119, AF-149, AF-150, AF-152, AF-154, AF-155, AF-156, AF-159, AF-160, AF-162, AF-163, AF-164, AF-169, AF-170, AF-172, AF-173, AF-174, AF-175, AF-176, AF-177, AF-178, AF-181, AF-183, AF-184, AF-186, AF-187, AF-188, AF-189, AF-190, AF-191 in any suitable combination. A decreased abundance of one or more in any suitable combination of such AFs in the CSF from the subject relative to CSF from a subject or subjects free from Alzheimer's disease (e.g., a control sample or a previously determined reference range) indicates the presence of Alzheimer's disease.

In another embodiment of the invention, CSF from a subject is analyzed by 2D electrophoresis for quantitative detection of one or more of the following AFs: AF-52, AF-53, AF-54, AF-55, AF-56, AF-57, AF-58, AF-59, AF-60, AF-61, AF-62, AF-63, AF-64, AF-65, AF-66, AF-67, AF-68, AF-69, AF-70, AF-71, AF-72, AF-73, AF-74, AF-75, AF-77, AF-121, AF-122, AF-123, AF-124, AF-125, AF-126, AF-127, AF-128, AF-129, AF-130, AF-131, AF-132, AF-133, AF-134, AF-131, AF-139, AF-140, AF-141, AF-142, AF-143, AF-144, AF-145, AF-146, AF-147, AF-148, AF-151, AF-153, AF-157, AF-161, AF-165, AF-166, AF-167, AF-168, AF-171, AF-179, AF-180, AF-182, AF-185, AF-192. An increased abundance of said one or more in any suitable combination of such AFs in the CSF from the subject relative to CSF from a subject or subjects free from Alzheimer's disease (e.g., a control sample or a previously determined reference range) indicates the presence of Alzheimer's disease.

In yet another embodiment, CSF from a subject is analyzed by 2D electrophoresis for quantitative detection of (a) one or more AFs or any suitable combination of them, whose decreased abundance indicates the presence of Alzheimer's Disease, i.e., AF-1, AF-2, AF-3, AF-4, AF-5, AF-6, AF-7, AF-8, AF-9, AF-10, AF-13, AF-14, AF-15, AF-16, AF-17, AF-18, AF-19, AF-20, AF-21, AF-22, AF-23, AF-24, AF-25, AF-26, AF-27, AF-28, AF-29, AF-30, AF-31, AF-32, AF-33, AF-34, AF-35, AF-36, AF-37, AF-38, AF-39, AF-40, AF-41, AF-42, AF-43, AF-44, AF-45, AF-46, AF-47, AF-48, AF-49, AF-50, AF-51, AF-76, AF-78, AF-79, AF-80, AF-81, AF-82, AF-83, AF-84, AF-85, AF-86, AF-87, AF-88, AF-89, AF-90, AF-91, AF-92, AF-93, AF-94, AF-95, AF-96, AF-98, AF-99, AF-100, AF-101, AF-102, AF-103, AF-104, AF-105, AF-107, AF-108, AF-110, AF-111, AF-112, AF-I14,AF-115, AF-116, AF-117, AF-118, AF-119, AF-149, AF-150, AF-152, AF-154, AF-155, AF-156, AF-159, AF-160, AF-162, AF-163, AF-164, AF-169, AF-170, AF-172, AF-173, AF-174, AF-175, AF-176, AF-177, AF-178, AF-181, AF-183, AF-184, AF-186, AF-187, AF-188, AF-189, AF-190, AF-191 and (b) one or more AFs or any combination of them, whose increased abundance indicates the presence of Alzheimer's Disease i.e., AF-52, AF-53, AF-54, AF-55, AF-56, AF-57, AF-58, AF-59, AF-60, AF-61, AF-62, AF-63, AF-64, AF-65, AF-66, AF-67, AF-68, AF-69, AF-70, AF-71, AF-72, AF-73, AF-74, AF-75, AF-77, AF-121, AF-122, AF-123, AF-124, AF-125, AF-126, AF-127, AF-128, AF-129, AF-130, AF-131, AF-132, AF-133, AF-134, AF-137, AF-139, AF-140, AF-141, AF-142, AF-143, AF-144, AF-145, AF-146, AF-147, AF-148, AF-151, AF-153, AF-157, AF-161, AF-165, AF-166, AF-167, AF-168, AF-171, AF-179, AF-180, AF-182, AF-185, AF-192.

In yet another embodiment of the invention, CSF from a subject is analyzed by 2D electrophoresis for quantitative detection of one or more of the following AFs: AF-1, AF-2, AF-3, AF-4, AF-5, AF-6, AF-7, AF-8, AF-9, AF-10, AF-13, AF-14, AF-15, AF-16, AF-17, AF-18, AF-19, AF-20, AF-21, AF-22, AF-23, AF-24, AF-25, AF-26, AF-27, AF-28, AF-29, AF-30, AF-31, AF-32, AF-33, AF-34, AF-35, AF-36, AF-37, AF-38, AF-39, AF-40, AF-41, AF-42, AF-43, AF-44, AF45, AF-46, AF-47, AF-48, AF-49, AF-50, AF-51, AF-52, AF-53, AF-54, AF-55, AF-56, AF-57, AF-58, AF-59, AF-60, AF-61, AF-62, AF-63, AF-64, AF-65, AF-66, AF-67, AF-68, AF-69, AF-70, AF-71, AF-72, AF-73, AF-74, AF-75, AF-76, AF-77, AF-78, AF-79, AF-80, AF-81, AF-82, AF-83, AF-84, AF-85, AF-86, AF-87, AF-88, AF-89, AF-90, AF-91, AF-92, AF-93, AF-94, AF-95, AF-96, AF-98, AF-99, AF-100, AF-100, AF-102, AF-103, AF-104, AF-105, AF-107, AF-108, AF-110, AF-111, AF-112, AF-114, AF-115, AF-116, AF-117, AF-118, AF- 19, AF-121, AF-122, AF-123, AF-124, AF-125, AF-126, AF-127, AF-128, AF-129, AF-130, AF-131, AF-132, AF-133, AF-134, AF-137, AF-139, AF-140, AF-141, AF-142, AF-143, AF-144, AF-145, AF-146, AF-147, AF-148, AF-149, AF-150, AF-151, AF-152, AF-153, AF-154, AF-155, AF-156, AF-157, AF-159, AF-160, AF-161, AF-162, AF-163, AF-164, AF-165, AF-166, AF-167, AF-168, AF-169, AF-170, AF-171, AF-172, AF-173, AF-174, AF-175,.AF-176, AF-177, AF-178, AF-179, AF-180, AF-181, AF-182, AF-183, AF-184, AF-186, AF-187, AF-188, AF-189, AF-190, AF-191, AF-185, AF-192, wherein the ratio of the one or more AFs relative to an Expression Reference Feature (ERF) indicates that Alzheimer's disease is present. In a specific embodiment, a decrease in one or more AF/ERF ratios in a sample being tested relative to the AF/ERF ratios in a control sample or a reference range indicates the presence of Alzheimer's disease; AF-1, AF-2, AF-3, AF-4, AF-5, AF-6, AF-7, AF-8, AF-9, AF-10, AF-13, AF-14, AF-15, AF-16, AF-17, AF-18, AF-19, AF-20, AF-21, AF-22, AF-23, AF-24, AF-25, AF-26, AF-27, AF-28, AF-29, AF-30, AF-31, AF-32, AF-33, AF-34, AF-35, AF-36, AF-37, AF-38, AF-39, AF-40, AF-41, AF-42, AF-43, AF-44, AF-45, AF-46, AF-47, AF-48, AF-49, AF-50, AF-51, AF-76, AF-78, AF-79, AF-80, AF-81, AF-82, AF-83, AF-84, AF-85, AF-86, AF-87, AF-88, AF-89, AF-90, AF-91, AF-92, AF-93, AF-94, AF-95, AF-96, AF-98, AF-99, AF-100, AF-101, AF-102, AF-103, AF-104, AF-105, AF-107, AF-108, AF-110, AF-111, AF-112, AF-114, AF-115, AF-116, AF-117, AF-118, AF- 119, AF-149, AF-150, AF-152, AF-154, AF-155, AF-156, AF-159, AF-160, AF-162, AF-163, AF-164, AF-169, AF-170, AF-172, AF-173, AF-174, AF-175, AF-176, AF-177, AF-178, AF-181, AF-183, AF-184, AF-186, AF-187, AF-188, AF-189, AF-190, AF-191 are suitable AFs for this purpose. In another specific embodiment, one may measure one or more Afs in a test sample, and compare them to an ERF, as a method for detecting the presence of Alzheimer's disease. Thus, an increase in one or more AF/ERF ratios in a test sample relative to the AF/ERF ratios in a control sample or a reference range indicates the presence of Alzheimer's disease; AF-52, AF-53, AF-54, AF-55, AF-56, AF-57, AF-58, AF-59, AF-60, AF-61, AF-62, AF-63, AF-64, AF-65, AF-66, AF-67, AF-68, AF-69, AF-70, AF-71, AF-72, AF-73, AF-74, AF-75, AF-121, AF-122, AF-123, AF-124, AF-125, AF-126, AF-127, AF-128, AF-129, AF-130, AF-131, AF-132, AF-133, AF-134, AF-137, AF-139, AF-140, AF-141, AF-142, AF-143, AF-i44, AF-145, AF-146, AF-147, AF-148, AF-151, AF-153, AF-157, AF-161, AF-165, AF-166, AF-167, AF-168, AF-171, AF-179, AF-180, AF-182, AF-185, AF-192 are suitable AFs for this purpose.

In a further embodiment of the invention, CSF from a subject is analyzed by 2D electrophoresis for quantitative detection of (a) one or more AFs, or any suitable combination of them, whose decreased AF/ERF ratio(s) in a test sample relative to the AF/ERF ratio(s) in a control sample indicates the presence of Alzheimer's Disease, i.e., AF-1, AF-2, AF-3, AF-4, AF-5, AF-6, AF-7, AF-8, AF-9, AF-10, AF-13, AF-14, AF-15, AF-16, AF-17, AF-18, AF-19, AF-20, AF-21, AF-22, AF-23, AF-24, AF-25, AF-26, AF-27, AF-28, AF-29, AF-30, AF-31, AF-32, AF-33, AF-34, AF-35, AF-36, AF-37, AF-38, AF-39, AF-40, AF-41, AF-42, AF-43, AF-44, AF-45, AF46, AF47, AF-48, AF-49, AF-50, AF-51, AF-76, AF-77, AF-78, AF-79, AF-80, AF-81, AF-82, AF-83, AF-84, AF-85, AF-86, AF-87, AF-88, AF-89, AF-90, AF-91, AF-92, AF-93, AF-94, AF-95, AF-96, AF-98, AF-99, AF-100, AF-101, AF-102, AF-103, AF-104, AF-105, AF-107, AF-108, AF-110, AF-111, AF-112, AF-114, AF-115, AF-116, AF-117, AF-118, AF-119, AF-149, AF-150, AF-152, AF-154, AF-155, AF-156, AF-159, AF-160, AF-162, AF-163, AF-164, AF-169, AF-170, AF-172, AF-173, AF-174, AF-175, AF-176, AF-177, AF-178, AF-181, AF-183, AF-184, AF-186, AF-187, AF-188, AF-189, AF-190, AF-191; (b) one or more AFs, or any combination of them, whose increased AF/ERF ratio(s) in a test sample relative to the AF/ERF ratio(s) in a control sample indicates the presence of Alzheimer's Disease, i.e., AF-52, AF-53, AF-54, AF-55, AF-56, AF-57, AF-58, AF-59, AF-60, AF-61, AF-62, AF-63, AF-64, AF-65, AF-66, AF-67, AF-68, AF-69, AF-70, AF-71, AF-72, AF-73, AF-74, AF-75, AF-77, AF-121, AF-122, AF-123, AF-124, AF-125, AF-126, AF-127, AF-128, AF-129, AF-130,AF-131,AF-132,AF-133,AF-134,AF-137,AF-139,AF-140,AF-141,AF-142, AF-143, AF-144, AF-145, AF-146, AF-147, AF-148, AF-151, AF-153, AF-157, AF-161, AF-165, AF-166, AF-167, AF-168, AF-171, AF-179, AF-180, AF-182, AF-185, AF-192.

In a preferred embodiment, CSF from a subject is analyzed for quantitative detection of a plurality of AFs.

Alzheimer's Disease-Associated Protein Isoforms (APIs)

In another aspect of the invention, CSF from a subject, is analyzed for quantitative detection of one or more Alzheimer's Disease-Associated Protein Isoforms (APIs), e.g. for screening, treatment or diagnosis of Alzheimer's Disease or for development of pharmaceutical products. As is well known in the art, a given protein may be expressed as one or more variants forms (isoforms) that differ in amino acid composition (e.g. as a result of alternative mRNA or premRNA processing, e.g. alternative splicing or limited proteolysis) or as a result of differential post-translational modification (e.g., glycosylation, phosphorylation, acylation), or both, so that proteins of identical amino acid sequence can differ in their pI, MW, or both. “Alzheimer's Disease-Associated Protein Isoform” refers to a protein isoform that is differentially present in CSF from a subject having Alzheimer's disease compared with CSF from a subject free from Alzheimer's disease.

Two groups of APIs are described herein by the amino acid sequencing of AFs. APIs were isolated, subjected to proteolysis, and analyzed by mass spectrometry using in this instance the methods and apparatus of the Preferred Technology, it being understood that the preferred technology is set forth as representative but not restrictive of the invention. One skilled in the art can identify sequence information from proteins analyzed by mass spectrometry and/or tandem mass spectrometry using various spectral interpretation methods and database searching tools. Examples of some of these methods and tools can be found at the Swiss Institute of Bioinformatics web site at http://www.expasy.ch/, and the European Molecular Biology Laboratory web site at www.mann.embl-heidelberg.de/Services/PeptideSearch/. Identification of APIs was performed using the SEQUEST search program (Eng et al., 1994, J. Am. Soc. Mass Spectrom. 5:976-989) with uninterpreted tandem mass spectra of tryptic digest peptides as described in the Examples, infra.

The first group comprises of APIs that are decreased in the CSF of subjects having Alzheimer's disease as compared with the CSF of subjects free from Alzheimer's disease. The amino acid sequences of peptides produced from these APIs by proteolysis using trypsin and identified by tandem mass spectrometry and database searching using the SEQUEST program are listed in Table IV, in addition to their corresponding pIs and MWs. For one API, the partial sequence information derived from tandem mass spectrometry was not found to be described in any known public database. This API is listed as ‘NOVEL’ in Table IV, and the partial amino acid sequence information derived from manually interpreting the MS/MS spectrum of tryptic peptides of this API as described in the Example infra, is given in Table IX. Table IV. APIs Decreased in CSF of Subjects having Alzheimer's disease

TABLE IV
APIs Decreased in CSF of Subjects having Alzheimer's disease

| | | Amino Acid Sequences of | | |
|---|---|---|---|---|
| AF# | API# | Tryptic Digest Peptides | pI | MW(Da) |
| AF-1 | API-47 | EDYICYAR, | 4.79 | 150081 |
| | | GKPPPSFSWTR, | | |
| | | QPEYAWQR | | |
| AF-1 | API-242 | IIMLFTDGGEER, | 4.79 | 150081 |
| | | FVVTDGGITR | | |
| AF-2 | API-1 | SGELEQEEER, | 4.28 | 21349 |
| | | EEEEEMAVVPQGLFR | | |
| AF-3 | API-48 | LVNIYDSMPLR, | 8.10 | 34846 |
| | | VIVVWNNIGEK, | | |
| | | YLELFQR | | |
| AF-5 | API-49 | DCSGVSLHLTR | 7.34 | 36554 |
| AF-6 | API-2 | TEAYLEAIR | 4.91 | 29812 |
| AF-8 | API-194 | DGNPFYFTDHR | 4.93 | 187927 |
| AF-9 | API-3 | AETYEGVYQCTAR | 5.21 | 136768 |
| AF-10 | API-50 | FWDYLR, | 5.19 | 17694 |
| | | GEVQAMLGQSTEELR | | |
| | | KVEQAVETEPEPELR | | |
| | | SELEEQLTPVAEETR | | |
| AF-10 | API-51 | VNSDGGLVALR | 5.19 | 17694 |
| AF-13 | API-4 | HYDGSYSTFGER, | 6.01 | 184530 |
| | | VGFYESDVMGR, | | |
| | | LPPNVVEESAR | | |
| AF-14 | API-52 | ADLSGITGAR, | 4.72 | 63166 |
| | | EIGELYLPK | | |
| AF-14 | API-243 | FEDGVLDPDYPR | 4.72 | 63166 |
| AF-15 | API-53 | ELDESLQVAER | 4.47 | 38970 |
| AF-15 | API-244 | TEVQLEHLSR | 4.47 | 38970 |
| AF-16 | API-54 | EGPVLILGR | 5.19 | 46876 |
| AF-17 | API-5 | EPGEFALLR, | 5.82 | 50294 |
| | | TALASGGVLDASGDYR, | | |
| | | YEAAVPDPR, | | |
| | | VAMHLVCPSR | | |
| AF-18 | API-55 | IVIGMDVAASEFYR, | 4.87 | 49219 |
| | | LGAEVYHTLK | | |
| AF-18 | API-245 | VEQATQAIPMER | 4.87 | 49219 |
| AF-21 | API-6 | LSPYVNYSFR, | 5.40 | 141094 |
| | | AETYEGVYQCTAR, | | |
| | | GKPPPSFSWTR, | | |
| | | IDGDTIIFSNVQER | | |
| AF-22 | API-56 | EGLDLQVLEDSGR, | 4.93 | 133773 |
| | | LICSELNGR, | | |
| | | RTMRDQDTGK | | |
| AF-22 | API-57 | YIFHNFMER, | 4.93 | 133773 |
| | | SPEQQETVLDGNLIIR, | | |
| | | NGIDIYSLTVDSR, | | |
| | | ILDDLSPR | | |
| AF-23 | API-7 | IPTTFENGR | 4.50 | 32473 |
| AF-23 | API-8 | EDEEEEEGENYQK, | 4.50 | 32473 |
| | | GEAGAPGEEDIQGPTK, | | |
| | | HLEEPGETQNAFLNER | | |
| AF-24 | API-9 | EGPVLILGR, | 5.31 | 46663 |
| | | IVQFSPSGK, | | |
| | | NNLVIFHR | | |
| AF-25 | API-10 | ASSIIDELFQDR | 5.68 | 36700 |
| AF-26 | API-14 | TMLLQPAGSLGSYSYR, | 8.11 | 32305 |
| | | APEAQVSVQPNFQQDK | | |
| AF-27 | API-15 | WLQGSQELPR | 5.33 | 141371 |
| AF-27 | API-58 | LSPYVNYSFR, | 5.33 | 141371 |
| | | AETYEGVYQCTAR, | | |
| | | GKPPPSFSWTR, | | |
| | | IDGDTIIFSNVQER, | | |
| | | NALGAIHHTISVR | | |
| AF-28 | API-16 | IALVITDGR | 5.13 | 158568 |
| AF-28 | API-59 | ALYLQYTDETFR, | 5.13 | 158568 |
| | | QSEDSTFYLGER, | | |
| | | GAYPLSIEPIGVR | | |
| AF-29 | API-196 | LVGGPMDASVEEEGVR | 9.22 | 47059 |
| | | ALDFAVGEYNK | | |
| AF-30 | API-17 | LAAAVSNFGYDLYR, | 5.67 | 48057 |
| | | TSLEDFYLDEER | | |
| AF-31 | API-60 | YIETDPANR, | 6.07 | 91258 |
| | | AGALNSNDAFVLK, | | |
| | | HVVPNEVVVQR | | |
| AF-32 | API-18 | EPGEFALLR, | 6.17 | 48958 |
| | | TALASGGVLDASGDYR, | | |
| | | VAMHLVCPSR | | |
| AF-34 | API-61 | TGLEAISNHK, | 4.54 | 145408 |
| | | FFEECDPNK | | |
| AF-35 | API-62 | QQTEWQSGQR, | 5.21 | 18623 |
| | | SELEEQLTPVAEETR | | |
| AF-37 | API-19 | DVIATDKEDVAFK, | 6.91 | 33523 |
| | | ENFSCLTR, | | |
| | | FVEGLPINDFSR, | | |
| | | EVGVYEALK | | |
| AF-38 | API-63 | LSELIQPLPLER, | 6.47 | 29535 |
| | | LVHGGPCDK, | | |
| | | EKPGVYTNVCR, | | |
| | | YTNWIQK | | |
| AF-39 | API-64 | CSVFYGAPSK | 7.50 | 35510 |
| AF-39 | API-65 | LVNIYDSMPLR, | 7.50 | 35510 |
| | | YLELFQR | | |
| AF-40 | API-20 | ITWSNPPAQGAR, | 7.29 | 38617 |
| | | VGGVQSLGGTGALR, | | |
| | | IGADFLAR, | | |
| | | NFGLYNER, | | |
| | | HIYLLPSGR | | |
| AF-41 | API-22 | LEGEACGVYTPR | 5.85 | 17345 |
| AF-42 | API-66 | LIVHNGYCDGR | 5.04 | 18662 |
| AF-43 | API-67 | LGPLVEQGR, | 9.83 | 14065 |
| | | LEEQAQQIR | | |
| AF-43 | API-68 | LVGGPMDASVEEEGVR | 9.83 | 14065 |
| AF-44 | API-69 | EELLPAQDIK | 6.63 | 102328 |
| AF-44 | API-70 | GCPTEEGCGER, | 6.63 | 102328 |
| | | AASGTQNNVLR | | |
| AF-45 | API-23 | ALYYDLISSPDIHGTYK, | 6.04 | 46998 |
| | | ELLDTVTAPQK, | | |
| | | LAAAVSNFGYDLYR, | | |
| | | TSLEDFYLDEER | | |
| AF-46 | API-24 | THPHFVIPYR | 4.71 | 19802 |
| AF-46 | API-197 | QSLEASLAETEGR, | 4.71 | 19802 |
| | | YENEVALR | | |
| AF-46 | API-198 | YEELQQTAGR | 4.71 | 19802 |
| AF-47 | API-25 | EPGEFALLR, | 5.99 | 49664 |
| | | TALASGGVLDASGDYR, | | |
| | | YEAAVPDPR, | | |
| | | VAMHLVCPSR | | |
| AF-48 | API-71 | YLELESSGHR, | 5.32 | 122332 |
| | | AFLFQESPR | | |
| AF-49 | API-26 | GLVSWGNIPCGSK, | 6.94 | 27576 |
| | | EKPGVYTNVCR | | |
| | | DSCQGDSGGPLVCGDHLR | | |
| AF-49 | API-27 | TMLLQPAGSLGSYSYR | 6.94 | 27576 |
| AF-50 | API-72 | NVPLPVIAELPPK | 6.82 | 71337 |
| AF-50 | API-73 | CFEPQLLR, | 6.82 | 71337 |
| | | EQPPSLTR | | |
| AF-50 | API-199 | YWNDCEPPDSR, | 6.82 | 71337 |
| | | DSPVLIDFFEDTER, | | |
| | | GGEGTGYFVDFSVR | | |
| AF-50 | API-200 | VYLFDFPEGK, | 6.82 | 71337 |
| | | CISIYSSER | | |
| AF-51 | API-28 | ASSIIDELFQDR | 5.70 | 34388 |
| AF-51 | API-30 | SADTLWDIQK, | 5.70 | 34388 |
| | | LKDDEVAQLK, | | |
| | | LIAPVAEEEATVPNNK | | |
| AF-76 | API-86 | EGPVLILGR, | 5.59 | 45537 |
| | | NNLVIFHR | | |
| AF-79 | API-201 | LPPNVVEESAR | 5.52 | 142378 |
| AF-81 | API-88 | LVESGGGLVQPGGSLR | 5.43 | 78299 |
| AF-81 | API-202 | GEASVCVEDWESGDR, | 5.43 | 78299 |
| | | VSSQNIQDFPSVLR | | |
| AF-82 | API-89 | LLEACTFHSAK, | 6.69 | 74838 |
| | | HSTVLENLPDK | | |
| AF-83 | API-90 | DQYELLCR, | 6.81 | 71920 |
| | | QMDFELLCQNGAR, | | |
| | | IECVSAENTEDCIAK, | | |
| | | SPDFQLFSSSHGK, | | |
| | | GSNFQWNQLQGK, | | |
| | | CGLVPVLAENYK, | | |
| | | WCTISNQEANK, | | |
| | | FDQFFGEGCAPGSQR, | | |
| | | EPVDNAENCHLAR, | | |
| | | WCAIGHEETQK, | | |
| | | HSTVLENLPDK | | |
| AF-84 | API-91 | DNPQTHYYAVAVVK, | 6.94 | 73402 |
| | | DQYELLCR, | | |
| | | QMDFELLCQNGAR, | | |
| | | VTCVAEELLK, | | |
| | | WCTISNQEANK, | | |
| | | EPVDNAENCHLAR, | | |
| | | FDQFFGEGCAPGSQR, | | |
| | | HSTVLENLPDK | | |
| AF-85 | API-92 | IPIEDGSGEVVLSR | 7.10 | 73878 |
| AF-85 | API-93 | DQYELLCR, | 7.10 | 73878 |
| | | FDQFFGEGCAPGSQR, | | |
| | | SPDFQLFSSSHGK, | | |
| | | EPVDNAENCHLAR, | | |
| | | CGLVPVLAENYK, | | |
| | | HSTVLENLPDK | | |
| AF-87 | API-95 | ECCHGDLLECADDR, | 5.95 | 64179 |
| | | IYEATLEDCCAK, | | |
| | | LGEYGFQNALIVR, | | |
| | | DVFLGTFLYEYSR, | | |
| | | FQPLVDEPK | | |
| AF-89 | API-97 | GYTQQLAFR, | 5.39 | 65155 |
| | | AGDFLEANYMNLQR | | |
| AF-90 | API-98 | LPLEYSYGEYR | 7.61 | 62945 |
| AF-91 | API-99 | LFEELVR, | 8.16 | 56352 |
| | | DPVQEAWAEDVDLR, | | |
| | | GIFPVLCK, | | |
| | | GDYPLEAVR | | |
| AF-100 | API-101 | LSCAEDYLSLVLNR, | 6.08 | 44068 |
| | | LGEYGFQNALIVR, | | |
| | | YICENQDTISTK, | | |
| | | CCTESLVNR, | | |
| | | DVFLGTFLYEYSR, | | |
| | | HPDYSVSLLLR | | |
| AF-103 | API-102 | INHGILYDEEK, | 5.93 | 42722 |
| | | EIMENYNIALR, | | |
| | | ITCTEEGWSPTPK | | |
| AF-104 | API-103 | YVMLPVADQEK | 5.09 | 42184 |
| AF-105 | API-104 | GSPAINVAVHVFR | 5.19 | 42184 |
| AF-107 | API-107 | ITVVDALHEIPVK, | 7.26 | 33226 |
| | | DNLAIQTR | | |
| AF-107 | API-210 | KLVVENVDVLTQMR | 7.26 | 33226 |
| AF-108 | API-108 | GYCAPGMECVK, | 7.54 | 33136 |
| | | GTCEQGPSIVTPPK, | | |
| | | AGAAAGGPGVSGVCVCK | | |
| AF-114 | API-111 | See Table IX | 6.80 | 18741 |
| | API-112 | | | |
| AF-117 | API-113 | KVEQAVETEPEPELR, | 4.65 | 13983 |
| | | SELEEQLTPVAEETR | | |
| AF-119 | API-114 | GTFATLSELHCDK, | 7.23 | 11699 |
| | | VVAGVANALAHK, | | |
| | | LLVVYPWTQR | | |
| AF-149 | API-214 | VEEVKPLEGR | 4.82 | 190721 |
| AF-150 | API-144 | GPPGPPGGVVVR, | 6.87 | 157592 |
| | | VEVLAGDLR | | |
| AF-152 | API-146 | FTFEYSR, | 5.04 | 81703 |
| | | FTDSENVCQER | | |
| AF-152 | API-147 | VIALINDQR | 5.04 | 81703 |
| AF-152 | API-148 | TATSEYQTFFNPR, | 5.04 | 81703 |
| | | ELLESYIDGR | | |
| AF-154 | API-150 | QEDDLANINQWVK, | 5.03 | 67307 |
| | | LCQDLGPGAFR | | |
| AF-154 | API-151 | DVVLTTTFVDDIK, | 5.03 | 67307 |
| | | AIEDYINEFSVR | | |
| AF-154 | API-152 | WLQGSQELPR | 5.03 | 67307 |
| AF-155 | API-215 | LVGGPMDASVEEEGVR, | 9.21 | 64021 |
| | | ALDFAVGEYNK | | |
| AF-156 | API-153 | DQDGEILLPR | 4.36 | 58083 |
| AF-159 | API-158 | TSLEDFYLDEER | 5.08 | 52008 |
| AF-159 | API-159 | EPGEFALLR, | 5.08 | 52008 |
| | | TALASGGVLDASGDYR | | |
| AF-159 | API-160 | YYTVFDR, | 5.08 | 52008 |
| | | QVFGEATK | | |
| AF-163 | API-165 | IPTTFENGR, | 4.45 | 34879 |
| | | CPNPPVQENFDVNK, | | |
| | | NILTSNNIDVK, | | |
| | | NPNLPPETVDSLK | | |
| AF-163 | API-166 | GEAGAPGEEDIQGPTK | 4.45 | 34879 |
| AF-164 | API-167 | ELDESLQVAER, | 5.00 | 33485 |
| | | FMETVAEK, | | |
| | | EILSVDCSTNNPSQAK | | |
| AF-169 | API-173 | LGQYASPTAK, | 8.00 | 34362 |
| | | GSFEFPVGDAVSK, | | |
| | | EELVYELNPLDHR | | |
| AF-170 | API-174 | ELDESLQVAER | 5.41 | 31886 |
| AF-170 | API-175 | GSPAINVAVHVFR, | 5.41 | 31886 |
| | | AADDTWEPFASGK | | |
| AF-170 | API-176 | SWFEPLVEDMQR, | 5.41 | 31886 |
| | | LGADMEDVCGR, | | |
| | | LEEQAQQIR, | | |
| | | SELEEQLTPVAEETR, | | |
| | | AATVGSLAGQPLQER | | |
| AF-172 | API-179 | GPCWCVDR, | 6.71 | 28747 |
| | | HLDSVLQQLQTEVYR | | |
| AF-172 | API-180 | KPNLQVFLGK, | 6.71 | 28747 |
| | | GLVSWGNIPCGSK, | | |
| | | EKPGVYTNVCR, | | |
| | | DSCQGDSGGPLVCGDHLR | | |
| AF-173 | API-181 | SNLDEDIIAEENIVSR, | 7.67 | 27476 |
| | | NEQVEIR | | |
| AF-174 | API-182 | SVTEQGAELSNEER | 4.67 | 27811 |
| AF-175 | API-183 | APEAQVSVQPNFQQDK, | 5.33 | 24936 |
| | | TMLLQPAGSLGSYSYR, | | |
| | | AQGFTEDTIVFLPQTDK | | |
| AF-176 | API-184 | TMLLQPAGSLGSYSYR, | 4.86 | 22248 |
| | | AQGFTEDTIVFLPQTDK | | |
| AF-178 | API-185 | LPFVINDGK | 6.03 | 22247 |
| AF-178 | API-217 | TMLLQPAGSLGSYSYR, | 6.03 | 22247 |
| | | AQGFTEDTIVFLPQTDK, | | |
| | | APEAQVSVQPNFQQDK | | |
| AF-178 | API-219 | TQGFTEDAIVFLPQTDK | 6.03 | 22247 |
| AF-181 | API-187 | HVGDLGNVTADK, | 5.72 | 16336 |
| | | GDGPVQGIINFEQK | | |
| AF-183 | API-189 | LVGGPMDASVEEEGVR, | 10.36 | 11160 |
| | | ALDFAVGEYNK | | |
| AF-184 | API-190 | ELLDTVTAPQK, | 5.31 | 48769 |
| | | TSLEDFYLDEER | | |
| AF-186 | API-238 | IPTTFENGR | 4.71 | 29693 |
| AF-187 | API-239 | QPEYAVVQR | 4.93 | 154156 |
| AF-190 | API-240 | ELDVLQGR, NNYMYAR | 5.29 | 29663 |

The second group comprises APIs that are increased in the CSF of subjects having Alzheimer's disease as compared with the CSF of subjects free from Alzheimer's disease. The amino acid sequences of peptides produced from these APIs by proteolysis using trypsin and identified by tandem mass spectrometry and database searching using the SEQUEST program are listed in Table V, in addition to their corresponding pIs and MWs.

TABLE V
APIs Increased In CSF of Subjects Having Alzheimer's Disease

| | | Amino Acid Sequences of | | |
|---|---|---|---|---|
| AF# | API# | Trypic Digest Peptides | pI | MW (Da) |
| AF-52 | API-74 | GLQDEDGYR, | 6.30 | 32573 |
| | | FACYYPR | | |
| AF-53 | API-33 | AVMDDFAAFVEK, | 5.84 | 45302 |
| | | YICENQDSISSK | | |
| AF-54 | API-221 | SELEEQLTPVAEETR | 5.12 | 17520 |
| AF-55 | API-34 | LVGGPMDASVEEEGVR, | 8.10 | 12361 |
| | | ALDFAVGEYNK | | |
| AF-56 | API-75 | NYCGLPGEYWLGNDK, | 8.56 | 52128 |
| | | IRPFFPQQ, | | |
| | | LESDVSAQMEYCR, | | |
| | | DNDGWLTSDPR | | |
| AF-56 | API-246 | AGALNSNDAFVLK, | 8.56 | 52128 |
| | | TGAQELLR | | |
| AF-57 | API-35 | MTLDDFR | 6.30 | 68549 |
| AF-57 | API-76 | VFLDCCNYITELR | 6.30 | 68549 |
| AF-57 | API-222 | QSLEASLAETEGR | 6.30 | 68549 |
| AF-58 | API-77 | KVEQAVETEPEPELR | 5.01 | 14507 |
| AF-59 | API-36 | TSLEDFYLDEER | 6.74 | 33401 |
| AF-60 | API-37 | GEVQAMLGQSTEELR, | 5.39 | 33873 |
| | | KVEQAVETEPEPELR, | | |
| | | SELEEQLTPVAEETR, | | |
| AF-61 | API-78 | QELSEAEQATR, | 6.76 | 54345 |
| | | TIYTPGSTVLYR, | | |
| | | IPIEDGSGEVVLSR | | |
| AF-62 | API-38 | GLQDEDGYR, | 6.60 | 31004 |
| | | ITQVLHFTK, | | |
| | | FACYYPR | | |
| AF-63 | API-79 | IWDVVEK, | 5.97 | 14897 |
| | | QPVPGQQMTLK, | | |
| | | EVVADSVWVDVK, | | |
| | | DSCVGSLVVK | | |
| AF-64 | API-80 | DFDFVPPVVR, | 6.67 | 68119 |
| | | SNLDEDIIAEENIVSR, | | |
| | | IPIEDGSGEVVLSR | | |
| AF-65 | API-81 | CLVNLIEK, | 7.19 | 58620 |
| | | FLCTGGVSPYADPNTCR | | |
| AF-65 | API-223 | VGDTLNLNLR | 7.19 | 58620 |
| AF-66 | API-82 | VFLDCCNYITELR, | 10.05 | 30092 |
| | | FISLGEACK | | |
| AF-66 | API-83 | LVGGPMDASVEEEGVR, | 10.05 | 30092 |
| | | ALDFAVGEYNK | | |
| AF-67 | API-39 | AADDTWEPFASGK | 5.02 | 13735 |
| AF-68 | API-84 | LISWYDNEFGYSNR, | 9.06 | 35351 |
| | | VPTANVSVVDLTCR, | | |
| AF-68 | API-85 | ISYQSSSTEER | 9.06 | 35351 |
| AF-69 | API-40 | TQVNTQAEQLR, | 5.01 | 46760 |
| | | ALVQQMEQLR | | |
| AF-69 | API-247 | VLSLAQEQVGGSPEK, | 5.01 | 46760 |
| | | AEMADQAAAWLTR, | | |
| | | QGSFQGGFR | | |
| AF-70 | API-41 | LVMGIPTFGR, | 8.91 | 38789 |
| | | EGDGSCFPDALDR, | | |
| | | FSNTDYAVGYMLR, | | |
| | | GNQWVGYDDQESVK, | | |
| | | QHFTTLIK | | |
| AF-70 | API-224 | DAIPEDLPPLTADFAEDK, | 8.91 | 38789 |
| | | YLYEIAR | | |
| AF-71 | API-42 | VFLDCCNYITELR, | 6.44 | 68579 |
| | | SNLDEDIIAEENIVSR, | | |
| | | GYTQQLAFR | | |
| AF-72 | API-43 | IDQTVEELR, | 5.00 | 43788 |
| | | TQVNTQAEQLR, | | |
| | | ALVQQMEQLR, | | |
| | | LEPYADQLR | | |
| AF-73 | API-44 | AADDTWEPFASGK | 5.21 | 31615 |
| AF-74 | API-45 | GECQAEGVLFFQGDR, | 6.19 | 51934 |
| | | YYCFQGNQFLR | | |
| AF-74 | API-248 | TIYTPGSTVLYR, | 6.19 | 51934 |
| | | TVMVNIENPEGIPVK | | |
| AF-75 | API-46 | ELDESLQVAER, | 5.03 | 33671 |
| | | EILSVDCSTNNPSQAK | | |
| AF-75 | API-225 | LGPLVEQGR, | 5.03 | 33671 |
| | | AATVGSLAGQPLQER | | |
| AF-121 | API-116 | DNCCILDER, | 5.42 | 105108 |
| | | YEASILTHDSSIR, | | |
| | | TSTADYAMFK, | | |
| | | VAQLEAQCQEPCK, | | |
| | | VELEDWNGR, | | |
| | | YLQEIYNSNNQK, | | |
| | | RLDGSVDFK, | | |
| AF-123 | API-118 | GLIDEVNQDFTNR, | 7.31 | 64933 |
| | | ADSGEGDFLAEGGGVR | | |
| AF-124 | API-119 | GLIDEVNQDFTNR, | 7.47 | 64736 |
| | | ESSSHHPGIAEFPSR | | |
| AF-125 | API-120 | SGNENGEFYLR | 4.77 | 61297 |
| AF-126 | API-121 | DQDGEILLPR, | 4.11 | 60374 |
| | | DCQPGLCCAFQR | | |
| AF-126 | API-122 | DQDGEILLPR | 4.11 | 60374 |
| AF-127 | API-123 | SLDFTELDVAAEK, | 4.98 | 59649 |
| | | ALQDQLVLVAAK | | |
| AF-128 | API-124 | LNMGITDLQGLR, | 6.60 | 57865 |
| | | VGDTLNLNLR | | |
| AF-129 | API-125 | KLCMAALK, | 5.29 | 54625 |
| | | ELPEHTVK, | | |
| | | THLPEVFLSK, | | |
| | | HLSLLTTLSNR, | | |
| | | FEDCCQEK, | | |
| | | LPEATPTELAK, | | |
| | | VCSQYAAYGEK, | | |
| | | YTFELSR, | | |
| | | LCDNLSTK | | |
| AF-129 | API-126 | SLDFTELDVAAEK, | 5.29 | 54625 |
| | | DPTFIPAPIQAK | | |
| AF-130 | API-127 | LQSLFDSPDFSK, | 5.08 | 51880 |
| | | LAAAVSNFGYDLYR, | | |
| | | TSLEDFYLDEER | | |
| AF-130 | API-128 | EPGEFALLR, | 5.08 | 51880 |
| | | TALASGGVLDASGDYR, | | |
| | | VAMHLVCPSR | | |
| AF-132 | API-130 | DHAVDLIQK, | 4.72 | 47414 |
| | | TEQWSTLPPETK, | | |
| | | VLSLAQEQVGGSPEK, | | |
| | | QGSFQGGFR, | | |
| | | ADGSYAAWLSR, | | |
| | | AEMADQASAWLTR | | |
| AF-133 | API-131 | TQVNTQAEQLR, | 5.12 | 44068 |
| | | LEPYADQLR | | |
| AF-134 | API-132 | LEPYADQLR | 5.00 | 43516 |
| AF-137 | API-134 | ELDESLQVAER, | 4.98 | 36855 |
| | | KYNELLK | | |
| AF-137 | API-135 | AQLGDLPWQVAIK, | 4.98 | 36855 |
| | | VFSLQWGEVK | | |
| AF-137 | API-232 | LGPIEAIQK | 4.98 | 36855 |
| AF-137 | API-233 | LGPLVEQGR, | 4.98 | 36855 |
| | | LEEQAQQIR | | |
| AF-137 | API-234 | KMEENEK | 4.98 | 36855 |
| AF-139 | API-136 | ELDESLQVAER, | 5.00 | 34295 |
| | | IDSLLENDR, | | |
| | | EDALNETRESETKLK, | | |
| | | EILSVDCSTNNPSQAK, | | |
| | | TLLSNLEEAK | | |
| AF-139 | API-137 | SELEEQLTPVAEETR, | 5.00 | 34295 |
| | | AATVGSLAGQPLQER | | |
| AF-140 | API-138 | GLQDEDGYR, | 6.80 | 32080 |
| | | FACYYPR | | |
| AF-141 | API-139 | LLEVPEGR, | 7.50 | 28440 |
| | | TNFDNDIALVR | | |
| AF-142 | API-140 | SNLDEDIIAEENIVSR, | 6.75 | 27279 |
| | | VELLHNPAFCSLATTK | | |
| AF-142 | API-141 | LSELIQPLPLER, | 6.75 | 27279 |
| AF-143 | API-142 | LLIYWASTR, | 7.44 | 26066 |
| | | SGTASVVCLLNNFYPR, | | |
| AF-144 | API-143 | EVDSGNDIYGNPIK, | 6.56 | 20744 |
| | | SDGSCAWYR | | |
| AF-151 | API-145 | AETYEGVYQCTAR, | 5.28 | 137531 |
| | | GKPPPSFSWTR, | | |
| | | IDGDTIIFSNVQER | | |
| AF-153 | API-149 | LNMGITDLQGLR, | 9.85 | 69630 |
| | | VGDTLNLNLR | | |
| AF-157 | API-155 | EPGEFALLR, | 4.99 | 55449 |
| | | TALASGGVLDASGDYR, | | |
| | | YEAAVPDPR | | |
| AF-161 | API-161 | IDQTVEELR, | 5.18 | 44404 |
| | | TQVNTQAEQLR, | | |
| | | SLAPYAQDTQEK, | | |
| | | ALVQQMEQLR, | | |
| | | LEPYADQLR, | | |
| | | RVEPYGENFNK | | |
| AF-161 | API-162 | TSLEDFYLDEER | 5.18 | 44404 |
| AF-161 | API-163 | AVFPSIVGR, | 5.18 | 44404 |
| | | SYELPDGQVITIGNER, | | |
| | | AGFAGDDAPR, | | |
| | | GYSFTTTAER, | | |
| | | QEYDESGPSIVHR, | | |
| | | VAPEEHPVLLTEAPLNPK | | |
| AF-165 | API-168 | EELVYELNPLDHR, | 7.17 | 34230 |
| | | EPFLSCCQFAESLR | | |
| AF-166 | API-169 | GLCVATPVQLR, | 8.54 | 33657 |
| | | EELVYELNPLDHR | | |
| AF-167 | API-170 | ASSIIDELFQDR, | 5.69 | 33621 |
| | | TLLSNLEEAK | | |
| AF-167 | API-171 | GEVQAMLGQSTEELRLEEQ | 5.69 | 33621 |
| | | AQQIR, | | |
| | | SELEEQLTPVAEETR | | |
| AF-168 | API-237 | ALEESNYELEGK | 7.66 | 33920 |
| AF-168 | API-172 | GSFEFPVGDAVSK, | 7.66 | 33920 |
| | | GLCVATPVQLR, | | |
| | | EELVYELNPLDHR, | | |
| | | EPFLSCCQFAESLR | | |
| AF-171 | API-177 | TMLLQPAGSLGSYSYR, | 4.98 | 29658 |
| | | AQGFTEDTIVFLPQTDK | | |
| AF-171 | API-178 | GSPAINVAVHVFR, | 4.98 | 29658 |
| | | AADDTWEPFASGK | | |
| AF-179 | API-186 | LIVHNGYCDGR, | 5.26 | 20115 |
| | | QEELCLAR, | | |
| | | FSGTWYAMAK | | |
| AF-180 | API-220 | CSVFYGAPSK, | 6.17 | 16255 |
| | | GLQDEDGYR | | |
| AF-182 | API-188 | AADDTWEPFASGK | 4.89 | 13651 |
| AF-185 | API-191 | VGYVSGWGR | 5.32 | 40323 |
| AF-185 | API-192 | SGNENGEFYLR, | 5.32 | 40323 |
| | | ADQVCINLR | | |

Those skilled in the art will understand, based upon the present description, that a given API can be described according to the data provided for that API in Table IV or V. The API is a protein comprising a peptide sequence described for that API (preferably comprising a plurality of, more preferably all of, the peptide sequences described for that API) and has a pI of about the value stated for that API (preferably within about 10%, more preferably within about 5% still more preferably within about 1% of the stated value) and has a MW of about the value stated for that API (preferably within about 10%, more preferably within about 5%, still more preferably within about 1% of the stated value).

In one embodiment, CSF from a subject is analyzed for quantitative detection of one or more of the following APIs: API-1, API-2, API-3, API-4, API-5, API-6, API-7, API-8, API-9, API-10, API-14, API-15, API-16, API-17, API-18, API-19, API-20, API-22, API-23, API-24, API-25, API-26, API-27, API-28, API-30, API47, API-48, API-49, API-50, API-51, API-52, API-53, API-54, API-55, API-56, API-57, API-58, API-59, API-60, API-61, API-62, API-63, API-64, API-65, API-66, API-67, API-68, API-69, API-70, API-71, API-72, API-73, API-86, API-88, API-89, API-90, API-91, API-92, API-93, API-95, API-97, API-98, API-99, API-I01, API-102, API-103, API-104, API-107, API-108, API-111, API-112, API-113, API-114, API-144, API-146, API-147, API-148, API-150, API-151, API-152, API-153, API-158, API-159, API-160, API-165, API-166, API-167, API-173, API-174, API-175, API-176, API-179, API-180, API-181, API-182, API-183, API-184, API-185, API-187, API-189, API-190, API-194, API-196, API-197, API-198, API-199, API-200, API-201, API-202, API-210, API-214, API-215, API-217, API-219, API-238, API-239, API-240, API-242, API-243, API-244, API-245 or any suitable combination of them, wherein a decreased abundance of the API or APIs (or any suitable combination of them) in the CSF from the subject relative to CSF from a subject or subjects free from Alzheimer's disease (e.g., a control sample or a previously determined reference range) indicates the presence of Alzheimer's disease.

In another embodiment of the invention, CSF from a subject is analyzed for quantitative detection of one or more of the following APIs: API-33, API-34, API-35, API-36, API-37, API-38, API-39, API-40, API-41, API-42, API-43, API44, API-45, API-46, API-74, API-75, API-76, API-77, API-78, API-79, API-80, API-81, API-82, API-83, API-84, API-85, API-116, API-118, API-119, API-120, API-121, API-122, API-123, API-124, API-125, API-126, API-127, API-128, API-130, API-131, API-132, API-134, API-135, API-136, API-137, API-138, API-139, API-140, API-141, API-142, API-143, API-145, API-149, API-155, API-161, API-162, API-163, API-168, API-169, API-170, API-171, API-172, API-177, API-178, API-186, API-188, API-191, API-192, API-220, API-221, API-222, API-223, API-224, API-225, API-232, API-233, API-234, API-237, API-246, API-247, API-248, or any suitable combination of them, wherein an increased abundance of the API or APIs (or any suitable combination of them) in CSF from the subject relative to CSF from a subject or subjects free from Alzheimer's disease (e.g., a control sample or a previously determined reference range) indicates the presence of Alzheimer's disease.

In a further embodiment, CSF from a subject is analyzed for quantitative detection of (a) one or more APIs, or any suitable combination of them, whose decreased abundance indicates the presence of Alzheimer's disease, ie., API-1, API-2, API-3, API4, API-5, API- 6, API-7, API-8, API-9, API-10, API-14, API-15, API-16, API-17, API-18, API-19, API-20, API-22, API-23, API-24, API-25, API-26, API-27, API-28, API-30, API-47, API-48, API-49, API-50, API-51, API-52, API-53, API-54, API-55, API-56, API-57, API-58, API-59, API-60, API-61, API-62, API-63, API-64, API-65, API-66, API-67, API-68, API-69, API-70, API-71, API-72, API-73, API-86, API-88, API-89, API-90, API-91, API-92, API-93, API-95, API-97, API-98, API-99, API-101, API-102, API-103, API-104, API-107, API-108, API-111, API-112, API-113, API-114, API-144, API-146, API-147, API-148, API-150, API-151, API-152, API-153, API-158, API-159, API-160, API-165, API-166, API-167, API-173, API-174, API-175, API-176, API-179, API-180, API-181, API-182, API-1 83, API-184, API-185, API-187, API-189, API-190, API-194, API-196, API-197, API-198, API-199, API-200, API-201, API-202, API-210, API-214, API-215, API-217, API-219, API-238, API-239, API-240, API-242, API-243, API-244, API-245; and one or more APIs, or any suitable combination of them, whose increased abundance indicates the presence of Alzheimer's disease, i.e., API-33, API-34, API-35, API-36, API-37, API-38, API-39, API-40, API-41, API-42, API-43, API-44, API-45, API-46, API-74, API-75, API-76, API-77, API-78, API-79, API-80, API-81, API-82, API-83, API-84, API-85, API-116, API-118, API-119, API-120, API-121, API-122, API-123, API-124, API-125, API-126, API-127, API-128, API-130, API-131, API-132, API-134, API-135, API-136, API-137, API-138, API-139, API-140, API-141, API-142, API-143, API-145, API-149, API-155, API-161, API-162, API-163, API-168, API-169, API-170, API-171, API-172, API-177, API-178, API-186, API-188, API-191, API-192, API-220, API-221, API-222, API-223, API-224, API-225, API-232, API-233, API-234, API-237, API-246, API-247, API-248.

In yet a further embodiment, CSF from a subject is analyzed for quantitative detection of one or more APIs and one or more previously known biomarkers of Alzheimer's disease (e.g., tau, NTP, Aβ2). In accordance with this embodiment, the abundance of each API and known biomarker relative to a control or reference range indicates whether a subject has Alzheimer's disease.

Preferably, the abundance of an API is normalized to an Expression Reference Protein Isoform (ERPI). ERPIs can be identified by partial amino acid sequence characterization of ERFs, which are described above, and which may be accomplished using e.g. the methods and apparatus of the Preferred Technology. The partial amino acid sequences of an ERPI is presented in Table VI.

TABLE VI

| | | Amino Acid Sequences of Tryptic |
|---|---|---|
| ERPI# | ERF# | Digest Peptides |
| ERPI-1 | ERF-2 | ELLDTVTAPQK, |
| | | LAAAVSNFGYDLYR, |
| | | TSLEDFYLDEER, |
| | | ALYYDLISSPDIHGTYK |

As shown above, the APIs described herein include previously unknown proteins, as well as isoforms of known proteins where the isoforms were not previously known to be associated with Alzheimer's disease. For each API, the present invention additionally provides: (a) a preparation comprising the isolated API; (b) a preparation comprising one or more fragments of an API; and (c) antibodies that bind to said API, to said fragments, or both to said API and to said fragments. As used herein, an API is “isolated” when it is present in a preparation that is substantially free of other proteins, i.e., a preparation in which less than 10% (particularly less than 5%, more particularly less than 1%) of the total protein present is contaminating protein(s). Another protein is a protein or protein isoform having a significantly different pI or MW from those of the isolated API, as determined by 2D electrophoresis. As used herein, a “significantly different” pI or MW is one that permits the other protein to be resolved from the API on 2D electrophoresis, performed according to the Reference Protocol.

In one embodiment, an isolated protein is provided, that comprises a peptide with the amino acid sequence identified in Table IV or V for an API, said protein having a pI and MW within 10% (particularly within 5%, more particularly within 1%) of the values identified in Table IV or V for that API.

The APIs of the invention can be qualitatively or quantitatively detected by any method known to those skilled in the art, including but not limited to the Preferred Technology described herein, kinase assays, enzyme assays, binding assays and other functional assays, immunoassays, and western blotting. In one embodiment, the APIs are separated on a 2-D gel by virtue of their MWs and pIs and are visualized by staining the gel. In one embodiment, the APIs are stained with a fluorescent dye and imaged with a fluorescence scanner. Sypro Red (Molecular Probes, Inc., Eugene, Oreg.) is a suitable dye for this purpose. A preferred fluorescent dye is Pyridinium, 4-[2-[4-(dipentylamino)-2-trifluoromethylphenyl]ethenyl]-1-(sulfobutyl)-, inner salt. See U.S. application Ser. No. 09/412,168, filed on Oct. 5, 1999, which is incorporated herein by reference in its entirety.

Alternatively, APIs can be detected in an immunoassay. In one embodiment, an immunoassay is performed by contacting a sample with an anti-API antibody under conditions such that immunospecific binding can occur if the API is present, and detecting or measuring the amount of any immunospecific binding by the antibody. Anti-API antibodies can be produced by the methods and techniques described herein; examples of such antibodies known in the art are set forth in Table VII. These antibodies shown in Table VII are already known to bind to the protein of which the API is itself a family member. Particularly, the anti-API antibody preferentially binds to the API rather than to other isoforms of the same protein. In a particular embodiment, the anti-API antibody binds to the API with at least 2-fold greater affinity, more particularly at least 5-fold greater affinity, still more preferably at least 10-fold greater affinity, than to said other isoforms of the same protein.

APIs can be transferred from a gel to a suitable membrane (e.g. a PVDF membrane) and subsequently probed in suitable assays that include, without limitation, competitive and non-competitive assay systems using techniques such as western blots and “sandwich” immunoassays using anti-API antibodies as described herein, e.g., the antibodies identified in Table VII, or others raised against the APIs of interest as those skilled in the art will appreciate based on the present description. The immunoblots can be used to identify those anti-API antibodies displaying the selectivity required to immuno-specifically differentiate an API from other isoforms encoded by the same gene.

TABLE VII
Known Antibodies That Recognize APIs or API-Related Polypeptides

| Protein family | | | |
|---|---|---|---|
| of which API | | | |
| is a member | Antibody | Manufacturer | Cat. No. |
| API-1 | Chromogranin A | BIODESIGN INTERNATIONAL | M54219M |
| API-3 | ANTI-Human CD56 ANTIGEN | RDI RESEARCH DIAGNOSTICS, | RDI-CBL159 |
| | (NEURAL CELL ADHESION | INC | |
| | MOLECULE) | | |
| API-4 | Gel | DAKO - 1998 CATALOGUE | A0033 |
| API-6 | ANTI-Human CD56 ANTIGEN | RDI RESEARCH DIAGNOSTICS, | RDI-CBL159 |
| | (NEURAL CELL ADHESION | INC | |
| | MOLECULE) | | |
| API-7 | Apolipoprotein D, Clone: | ACCURATE CHEMICAL & | MED-CLA457 |
| | 36C6, Mab anti-Human, | SCIENTIFIC CORPORATION | |
| | paraffin, IH/WB | | |
| API-10 | Goat anti-Clusterin (human) | RDI RESEARCH DIAGNOSTICS, | RDI- |
| | | INC | CLUSTRCabG |
| API-15 | Monoclonal mouse anti- | RDI RESEARCH DIAGNOSTICS, | RDI-TRK1A2- |
| | human IgA1 | INC | 2B5 |
| API-16 | Monoclonal anti Human | BIODESIGN INTERNATIONAL | M22090M |
| | Collagen Type VI | | |
| API-22 | RABBIT anti-human INSULIN | RDI RESEARCH DIAGNOSTICS, | RDI-IGFBP2abr |
| | GROWTH FACTOR BINDING | INC | |
| | PROTEIN 2 | | |
| API-28 | Goat anti-Clusterin (human) | RDI RESEARCH DIAGNOSTICS, | RDI- |
| | | INC | CLUSTRCabG |
| API-30 | Lactic Dehydrogenase (LDH) | ACCURATE CHEMICAL & | BYA-6019-1 |
| | (H-subunit), Clone: HH-17, | SCIENTIFIC CORPORATION | |
| | Mab anti-Human | | |
| API-33 | Albumin, Human, Chicken | ACCURATE CHEMICAL & | IMS-01-026-02 |
| | anti- | SCIENTIFIC CORPORATION | |
| API-34 | Cystatin C, Rabbit anti-Human | ACCURATE CHEMICAL & | AXL-574 |
| | | SCIENTIFIC CORPORATION | |
| API-37 | Apolipoprotein E, LDL, VLDL, | ACCURATE CHEMICAL & | YM-5029 |
| | Clone: 3D12, Mab anti- | SCIENTIFIC CORPORATION | |
| | Human, frozen/paraffin | | |
| API-38 | C4 Complement, Chicken | ACCURATE CHEMICAL & | IMS-01-032-02 |
| | anti-Human | SCIENTIFIC CORPORATION | |
| API-39 | Transthyretin, Prealbuminm, | ACCURATE CHEMICAL & | MED-CLA 193 |
| | 55 kD, Rabbit anti-Human | SCIENTIFIC CORPORATION | |
| API-40 | Apolipoprotein A (HDL), | ACCURATE CHEMICAL & | ACL-20076A |
| | Plasminogen absorbed, | SCIENTIFIC CORPORATION | |
| | Sheep anti-Human | | |
| API-42 | C3 Complement, Chicken | ACCURATE CHEMICAL & | IMS-01-001-02 |
| | anti-Human | SCIENTIFIC CORPORATION | |
| API-43 | Apolipoprotein A (HDL), | ACCURATE CHEMICAL & | ACL-20076A |
| | Plasminogen absorbed, | SCIENTIFIC CORPORATION | |
| | Sheep anti-Human | | |
| API-44 | Transthyretin, Prealbuminm, | ACCURATE CHEMICAL & | MED-CLA 193 |
| | 55 kD, Rabbit anti-Human | SCIENTIFIC CORPORATION | |
| API-45 | Hemopexin, Beta-1, Rabbit | ACCURATE CHEMICAL & | YN-RHHPX |
| | anti-Human, precipitating | SCIENTIFIC CORPORATION | |
| API-46 | Goat anti-Clusterin (human) | RDI RESEARCH DIAGNOSTICS, | RDI- |
| | | INC | CLUSTRCabG |
| API-47 | ANTI-Human CD56 ANTIGEN | RDI RESEARCH DIAGNOSTICS, | RDI-CBL159 |
| | (NEURAL CELL ADHESION | INC | |
| | MOLECULE) | | |
| API-50 | Apolipoprotein E, LDL, VLDL, | ACCURATE CHEMICAL & | YM-5029 |
| | Clone: 3D12, Mab anti- | SCIENTIFIC CORPORATION | |
| | Human, frozen/paraffin | | |
| API-52 | Alpha-1-Antichymotrypsin, | ACCURATE CHEMICAL & | AXL-145/2 |
| | Rabbit anti-Human | SCIENTIFIC CORPORATION | |
| API-53 | Goat anti-Clusterin (human) | RDI RESEARCH DIAGNOSTICS, | RDI- |
| | | INC | CLUSTRCabG |
| API-55 | Monoclonal anti-Neuron | BIODESIGN INTERNATIONAL | M37403M |
| | Specific Enolase | | |
| API-58 | ANTI-Human CD56 ANTIGEN | RDI RESEARCH DIAGNOSTICS, | RDI-CBL159 |
| | (NEURAL CELL ADHESION | INC | |
| | MOLECULE) | | |
| API-60 | Gelsolin, plasma + cytoplasmic, | ACCURATE CHEMICAL & | YBG-4628-6210 |
| | Sheep anti- | SCIENTIFIC CORPORATION | |
| API-62 | Apolipoprotein E, LDL, VLDL, | ACCURATE CHEMICAL & | YM-5029 |
| | Clone: 3D12, Mab anti- | SCIENTIFIC CORPORATION | |
| | Human, frozen/paraffin | | |
| API-64 | C4 Complement, Chicken | ACCURATE CHEMICAL & | IMS-01-032-02 |
| | anti-Human | SCIENTIFIC CORPORATION | |
| API-66 | Retinal Binding Protein, | ACCURATE CHEMICAL & | AXL-163/2 |
| | Rabbit anti-Human | SCIENTIFIC CORPORATION | |
| API-67 | Apolipoprotein E, LDL, VLDL, | ACCURATE CHEMICAL & | YM-5029 |
| | Clone: 3D12, Mab anti- | SCIENTIFIC CORPORATION | |
| | Human, frozen/paraffin | | |
| API-69 | Complement Factor B, C3 | ACCURATE CHEMICAL & | AXL-466/2 |
| | proactivator, Rabbit anti- | SCIENTIFIC CORPORATION | |
| | Human | | |
| API-72 | Gel | DAKO - 1998 CATALOGUE | A0475 |
| API-74 | C4 Complement, Chicken | ACCURATE CHEMICAL & | IMS-01-032-02 |
| | anti-Human | SCIENTIFIC CORPORATION | |
| API-75 | Fibrinogen, Fibrin I, B-beta | ACCURATE CHEMICAL & | NYB-18C6 |
| | chain (Bβ 1-42), Clone: 18C6, | SCIENTIFIC CORPORATION | |
| | Mab anti-Human | | |
| API-76 | C3 Complement, Chicken | ACCURATE CHEMICAL & | IMS-01-001-02 |
| | anti-Human | SCIENTIFIC CORPORATION | |
| API-77 | Apolipoprotein E, LDL, VLDL, | ACCURATE CHEMICAL & | YM-5029 |
| | Clone: 3D12, Mab anti- | SCIENTIFIC CORPORATION | |
| | Human, frozen/paraffin | | |
| API-78 | C3 Complement, Chicken | ACCURATE CHEMICAL & | IMS-01-001-02 |
| | anti-Human | SCIENTIFIC CORPORATION | |
| API-79 | C3 Complement, Chicken | ACCURATE CHEMICAL & | IMS-01-001-02 |
| | anti-Human | SCIENTIFIC CORPORATION | |
| API-80 | C3 Complement, Chicken | ACCURATE CHEMICAL & | IMS-01-001-02 |
| | anti-Human | SCIENTIFIC CORPORATION | |
| API-81 | Complement Factor B, C3 | ACCURATE CHEMICAL & | AXL-466/2 |
| | proactivator, Rabbit anti- | SCIENTIFIC CORPORATION | |
| | Human | | |
| API-82 | C3 Complement, Chicken | ACCURATE CHEMICAL & | IMS-01-001-02 |
| | anti-Human | SCIENTIFIC CORPORATION | |
| API-84 | Glyceraldehyde-3-Phosphate | BIODESIGN INTERNATIONAL | H86504M |
| | Dehydrogenase | | |
| API-90 | Monoclonal mouse anti- | RDI RESEARCH DIAGNOSTICS, | RDI-TRK4L2- |
| | lactoferrin | INC | LF2B8 |
| API-92 | C3 Complement, Chicken | ACCURATE CHEMICAL & | IMS-01-001-02 |
| | anti-Human | SCIENTIFIC CORPORATION | |
| API-93 | Monoclonal mouse anti- | RDI RESEARCH DIAGNOSTICS, | RDI-TRK4L2- |
| | lactoferrin | INC | LF2B8 |
| API-95 | Albumin, Human, Chicken | ACCURATE CHEMICAL & | IMS-01-026-02 |
| | anti- | SCIENTIFIC CORPORATION | |
| API-97 | C3 Complement, Chicken | ACCURATE CHEMICAL & | IMS-01-001-02 |
| | anti-Human | SCIENTIFIC CORPORATION | |
| API-98 | C8 Complement, Goat anti- | ACCURATE CHEMICAL & | BMD-G35 |
| | Human | SCIENTIFIC CORPORATION | |
| API-101 | Albumin, Human, Chicken | ACCURATE CHEMICAL & | IMS-01-026-02 |
| | anti- | SCIENTIFIC CORPORATION | |
| API-102 | Factor H (Complement), | ACCURATE CHEMICAL & | IMS-01-066-02 |
| | Chicken anti-Human | SCIENTIFIC CORPORATION | |
| API-103 | Goat anti-Haptoglobin | BIODESIGN INTERNATIONAL | L15320G |
| API-104 | Transthyretin, Prealbuminm, | ACCURATE CHEMICAL & | MED-CLA 193 |
| | 55 kD, Rabbit anti-Human | SCIENTIFIC CORPORATION | |
| API-113 | Apolipoprotein E, LDL, VLDL, | ACCURATE CHEMICAL & | YM-5029 |
| | Clone: 3D12, Mab anti- | SCIENTIFIC CORPORATION | |
| | Human, frozen/paraffin | | |
| API-118 | Monoclonal anti-human | BIODESIGN INTERNATIONAL | N77190M |
| | Fibrinogen | | |
| API-119 | Monoclonal anti-human | BIODESIGN INTERNATIONAL | N77190M |
| | Fibrinogen | | |
| API-123 | AT1 (306) | SANTA CRUZ | sc-579 |
| | | BIOTECHNOLOGY, INC - | |
| | | RESEARCH ANTIBODIES 98/99 | |
| API-124 | C4 Complement, Chicken | ACCURATE CHEMICAL & | IMS-01-032-02 |
| | anti-Human | SCIENTIFIC CORPORATION | |
| API-126 | AT1 (306) | SANTA CRUZ | sc-579 |
| | | BIOTECHNOLOGY, INC - | |
| | | RESEARCH ANTIBODIES 98/99 | |
| API-130 | C4 Complement, Chicken | ACCURATE CHEMICAL & | IMS-01-032-02 |
| | anti-Human | SCIENTIFIC CORPORATION | |
| API-131 | Apolipoprotein A (HDL), | ACCURATE CHEMICAL & | ACL-20076A |
| | Plasminogen absorbed, | SCIENTIFIC CORPORATION | |
| | Sheep anti-Human | | |
| API-132 | Apolipoprotein A (HDL), | ACCURATE CHEMICAL & | ACL-20076A |
| | Plasminogen absorbed, | SCIENTIFIC CORPORATION | |
| | Sheep anti-Human | | |
| API-134 | Goat anti-Clusterin (human) | RDI RESEARCH DIAGNOSTICS, | RDI- |
| | | INC | CLUSTRCabG |
| API-136 | Goat anti-Clusterin (human) | RDI RESEARCH DIAGNOSTICS, | RDI- |
| | | INC | CLUSTRCabG |
| API-137 | Apolipoprotein E, LDL, VLDL, | ACCURATE CHEMICAL & | YM-5029 |
| | Clone: 3D12, Mab anti- | SCIENTIFIC CORPORATION | |
| | Human, frozen/paraffin | | |
| API-138 | C4 Complement, Chicken | ACCURATE CHEMICAL & | IMS-01-032-02 |
| | anti-Human | SCIENTIFIC CORPORATION | |
| API-140 | C3 Complement, Chicken | ACCURATE CHEMICAL & | IMS-01-001-02 |
| | anti-Human | SCIENTIFIC CORPORATION | |
| API-142 | Kappa Chain, Mab anti- | ACCURATE CHEMICAL & | BMD-021D |
| | Human | SCIENTIFIC CORPORATION | |
| API-143 | Tissue Inhibitor of Matrix | ACCURATE CHEMICAL & | MED-CLA498 |
| | Metalloproteinase 2 (TIMP2) | SCIENTIFIC CORPORATION | |
| | (NO X w/TIMP1), Clone: 3A4, | | |
| | Mab anti-Human, paraffin, IH | | |
| API-145 | ANTI-Human CD56 ANTIGEN | RDI RESEARCH DIAGNOSTICS, | RDI-CBL159 |
| | (NEURAL CELL ADHESION | INC | |
| | MOLECULE) | | |
| API-149 | C4 Complement, Chicken | ACCURATE CHEMICAL & | IMS-01-032-02 |
| | anti-Human | SCIENTIFIC CORPORATION | |
| API-150 | Sheep anti-Alpha 2 | BIODESIGN INTERNATIONAL | K90038C |
| | Antiplasmin | | |
| API-161 | Apolipoprotein A (HDL), | ACCURATE CHEMICAL & | ACL-20076A |
| | Plasminogen absorbed, | SCIENTIFIC CORPORATION | |
| | Sheep anti-Human | | |
| API-165 | Apolipoprotein D, Clone: | ACCURATE CHEMICAL & | MED-CLA457 |
| | 36C6, Mab anti-Human, | SCIENTIFIC CORPORATION | |
| | paraffin, IH/WB | | |
| API-167 | Goat anti-Clusterin (human) | RDI RESEARCH DIAGNOSTICS, | RDI- |
| | | INC | CLUSTRCabG |
| API-168 | C4 Complement, Chicken | ACCURATE CHEMICAL & | IMS-01-032-02 |
| | anti-Human | SCIENTIFIC CORPORATION | |
| API-169 | C4 Complement, Chicken | ACCURATE CHEMICAL & | IMS-01-032-02 |
| | anti-Human | SCIENTIFIC CORPORATION | |
| API-170 | Goat anti-Clusterin (human) | RDI RESEARCH DIAGNOSTICS, | RDI- |
| | | INC | CLUSTRCabG |
| API-171 | Apolipoprotein E, LDL, VLDL, | ACCURATE CHEMICAL & | YM-5029 |
| | Clone: 3D12, Mab anti- | SCIENTIFIC CORPORATION | |
| | Human, frozen/paraffin | | |
| API-172 | C4 Complement, Chicken | ACCURATE CHEMICAL & | IMS-01-032-02 |
| | anti-Human | SCIENTIFIC CORPORATION | |
| API-173 | C4 Complement, Chicken | ACCURATE CHEMICAL & | IMS-01-032-02 |
| | anti-Human | SCIENTIFIC CORPORATION | |
| API-174 | Goat anti-Clusterin (human) | RDI RESEARCH DIAGNOSTICS, | RDI- |
| | | INC | CLUSTRCabG |
| API-175 | Transthyretin, Prealbuminm, | ACCURATE CHEMICAL & | MED-CLA 193 |
| | 55 kD, Rabbit anti-Human | SCIENTIFIC CORPORATION | |
| API-176 | Apolipoprotein E, LDL, VLDL, | ACCURATE CHEMICAL & | YM-5029 |
| | Clone: 3D12, Mab anti- | SCIENTIFIC CORPORATION | |
| | Human, frozen/paraffin | | |
| API-178 | Transthyretin, Prealbuminm, | ACCURATE CHEMICAL & | MED-CLA 193 |
| | 55 kD, Rabbit anti-Human | SCIENTIFIC CORPORATION | |
| API-179 | IGFBP6 (M-20) | SANTA CRUZ | sc-6008 |
| | | BIOTECHNOLOGY, INC- | |
| | | RESEARCH ANTIBODIES 98/99 | |
| API-181 | C3 Complement, Chicken | ACCURATE CHEMICAL & | IMS-01-001-02 |
| | anti-Human | SCIENTIFIC CORPORATION | |
| API-182 | Rabbit anti-14-3-3B (Broadly | RDI RESEARCH DIAGNOSTICS, | RDI-1433BNabr |
| | Reactive) | INC | |
| API-186 | Retinol Binding Protein, | ACCURATE CHEMICAL & | AXL-163/2 |
| | Rabbit anti-Human | SCIENTIFIC CORPORATION | |
| API-187 | Anti-Superoxide Dismutase | RDI RESEARCH DIAGNOSTICS, | RDI-SODabg |
| | (Cu/Zn-SOD) IgG fraction | INC | |
| | (POLYCLONAL) | | |
| API-188 | Transthyretin, Prealbuminm, | ACCURATE CHEMICAL & | MED-CLA 193 |
| | 55 kD, Rabbit anti-Human | SCIENTIFIC CORPORATION | |
| API-189 | Cystatin C, Rabbit anti-Human | ACCURATE CHEMICAL & | AXL-574 |
| | | SCIENTIFIC CORPORATION | |
| API-191 | Goat anti-Haptoglobin | BIODESIGN INTERNATIONAL | L15320G |
| API-194 | ANTI-Human CD56 ANTIGEN | RDI RESEARCH DIAGNOSTICS, | RDI-CBL159 |
| | (NEURAL CELL ADHESION | INC | |
| | MOLECULE) | | |
| API-196 | Cystatin C, Rabbit anti-Human | ACCURATE CHEMICAL & | AXL-574 |
| | | SCIENTIFIC CORPORATION | |
| API-201 | Gel | DAKO - 1998 CATALOGUE | A0033 |
| API-215 | Cystatin C, Rabbit anti-Human | ACCURATE CHEMICAL & | AXL-574 |
| | | SCIENTIFIC CORPORATION | |
| API-220 | C4 Complement, Chicken | ACCURATE CHEMICAL & | IMS-01-032-02 |
| | anti-Human | SCIENTIFIC CORPORATION | |
| API-221 | Apolipoprotein E, LDL, VLDL, | ACCURATE CHEMICAL & | YM-5029 |
| | Clone: 3D12, Mab anti- | SCIENTIFIC CORPORATION | |
| | Human, frozen/paraffin | | |
| API-223 | C4 Complement, Chicken | ACCURATE CHEMICAL & | IMS-01-032-02 |
| | anti-Human | SCIENTIFIC CORPORATION | |
| API-225 | Apolipoprotein E, LDL, VLDL, | ACCURATE CHEMICAL & | YM-5029 |
| | Clone: 3D12, Mab anti- | SCIENTIFIC CORPORATION | |
| | Human, frozen/paraffin | | |
| API-233 | Apolipoprotein E, LDL, VLDL, | ACCURATE CHEMICAL & | YM-5029 |
| | Clone: 3D12, Mab anti- | SCIENTIFIC CORPORATION | |
| | Human, frozen/paraffin | | |
| API-238 | Apolipoprotein D, Clone: | ACCURATE CHEMICAL & | MED-CLA457 |
| | 36C6, Mab anti-Human, | SCIENTIFIC CORPORATION | |
| | paraffin, IH/WB | | |
| API-239 | ANTI-Human CD56 ANTIGEN | RDI RESEARCH DIAGNOSTICS, | RDI-CBL159 |
| | (NEURAL CELL ADHESION | INC | |
| | MOLECULE) | | |

In one embodiment, binding of antibody in tissue sections can be used to detect API localization or the level of one or more APIs. In a specific embodiment, antibody to an API can be used to assay a tissue sample (e.g., a brain biopsy) from a subject for the level of the API where a substantially changed level of API is indicative of Alzheimer's disease. As used herein, a “substantially changed level” means a level that is increased or decreased compared with the level in a subject free from Alzheimer's disease or a reference level. If desired, the comparison can be performed with a matched sample from the same subject, taken from a portion of the body not affected by Alzheimer's disease.

Any suitable immunoassay can be used to detect an API, including, without limitation, competitive and non-competitive assay systems using techniques such as western blots, radioimmunoassays, ELISAs (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, an API can be detected in a fluid sample (e.g., CSF, blood, urine, or tissue homogenate) by means of a two-step sandwich assay. In the first step, a capture reagent (e.g., an anti-API antibody) is used to capture the API. Examples of such antibodies known in the art are set forth in Table VII. The capture reagent can optionally be immobilized on a solid phase. In the second step, a directly or indirectly labeled detection reagent is used to detect the captured API. In one embodiment, the detection reagent is a lectin. A lectin can be used for this purpose that preferentially binds to the API rather than to other isoforms that have the same core protein as the API or to other proteins that share the antigenic determinant recognized by the antibody. In a preferred embodiment, the chosen lectin binds to the API with at least 2-fold greater affinity, more preferably at least 5-fold greater affinity, still more preferably at least 10-fold greater affinity, than to said other isoforms that have the same core protein as the API or to said other proteins that share the antigenic. determinant recognized by the antibody. Based on the present description, a lectin that is suitable for detecting a given API can readily be identified by those skilled in the art using methods well known in the art, for instance upon testing one or more lectins enumerated in Table I on pages 158-159 of Sumar et al., Lectins as Indicators of Disease-Associated Glycoforms, In: Gabius H-J & Gabius S (eds.), 1993, Lectins and Glycobiology, at pp. 158-174 (which is incorporated herein by reference in its entirety). Lectins with the desired oligosaccharide specificity can be identified, for example, by their ability to detect the API in a 2D gel, in a replica of a 2D gel following transfer to a suitable solid substrate such as a nitrocellulose membrane, or in a two-step assay following capture by an antibody. In an alternative embodiment, the detection reagent is an antibody, e.g., an antibody that immunospecifically detects other post-translational modifications, such as an antibody that immunospecifically binds to phosphorylated amino acids. Examples of such antibodies include those that bind to phosphotyrosine (BD Transduction Laboratories, catalog nos.: P11230-050/P1 1230-150; P11120; P38820; P39020), those that bind to phosphoserine (Zymed Laboratories Inc., South San Francisco, Calif., catalog no. 61-8100) and those that bind to phosphothreonine (Zymed Laboratories Inc., South San Francisco, Calif., catalog nos. 71-8200, 13-9200).

If desired, a gene encoding an API, a related gene (e.g. a gene having sequence homology), or related nucleic acid sequences or subsequences, including complementary sequences, can also be used in hybridization assays. A nucleotide encoding an API, or subsequences thereof comprising at least 8 nucleotides, preferably at least 12 nucleotides, and most preferably at least 15 nucleotides can be used as a hybridization probe. Hybridization assays can be used for detection, treatment, diagnosis, or monitoring of conditions, disorders, or disease states, associated with aberrant expression of genes encoding APIs, or for differential diagnosis of subjects with signs or symptoms suggestive of Alzheimer's disease. In particular, such a hybridization assay can be carried out by a method comprising contacting a subject's sample containing nucleic acid with a nucleic acid probe capable of hybridizing to a DNA or RNA that encodes an API, under conditions such that hybridization can occur, and detecting or measuring any resulting hybridization. Nucleotides can be used for therapy of subjects having Alzheimer's disease, as described below.

The invention also provides diagnostic kits, comprising an anti-API antibody. In addition, such a kit may optionally comprise one or more of the following: (1) instructions for using the anti-API antibody for diagnosis, prognosis, therapeutic monitoring or any suitable combination of these applications; (2) a labeled binding partner to the antibody; (3) a solid phase (such as a reagent strip) upon which the anti-API antibody is immobilized; and (4) a label or insert indicating regulatory approval for diagnostic, prognostic or therapeutic use or any suitable combination thereof If no labeled binding partner to the antibody is provided, the anti-API antibody itself can be labeled with a detectable marker, e.g., a chemiluminescent, enzymatic, fluorescent, or radioactive moiety.

The invention also provides a kit comprising a nucleic acid probe capable of hybridizing to RNA encoding an API. In a specific embodiment, a kit comprises in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding an API, such as by polymerase chain reaction (see, e.g., Innis et al., 1990, PCR Protocols, Academic Press, Inc., San Diego, Calif.), ligase chain reaction (see EP 320,308) use of Qβ replicase, cyclic probe reaction, or other methods known in the art.

Kits are also provided which allow for the detection of a plurality of APIs or a plurality of nucleic acids each encoding an API. A kit can optionally further comprise a predetermined amount of an isolated API protein or a nucleic acid encoding an API, e.g., for use as a standard or control.

Statistical Techniques for Identifying APIs and API Clusters

Uni-variate differential analysis tools, such as fold changes, wilcoxon rank sum test and t-test, are useful in identifying individual AFs or APIs that are diagnostically associated with Alzheimer's disease or in identifying individual APIs that regulate the disease process. However, those skilled in the art will appreciate that the disease process is associated with a suitable combination of AFs or APIs (and to be regulated by a suitable combination of APIs), rather than individual AFs and APIs in isolation. The strategies for discovering such suitable combinations of AFs and APIs differ from those for discovering individual AFs and APIs. In such cases, each individual AF and API can be regarded as one variable and the disease can be regarded as a joint, multi-variate effect caused by interaction of these variables.

The following steps can be used to identify markers from data produced by the Preferred Technology.

The first step is to identify a collection of AFs or APIs that individually show significant association with Alzheimer's disease. The association between the identified individual AFs or individual APIs and AD need not be as highly significant when a collection of AFs and APIs as is desirable when an individual AF or API is used as a diagnostic. Any of the tests discussed above (fold changes, wilcoxon rank sum test, etc.) can be used at this stage. Once a suitable collection of AFs or APIs has been identified, a sophisticated multi-variate analysis capable of identifying clusters can then be used to estimate the significant multivariate associations with Alzheimer's disease.

Linear Discriminant Analysis (LDA) is one such procedure, which can be used to detect significant association between a cluster of variables (ie., AFs or APIs) and Alzheimer's disease. In performing LDA, a set of weights is associated with each variable (i.e., AF or API) so that the linear combination of weights and the measured values of the variables can identify the disease state by discriminating between subjects having Alzheimer's disease and subjects free from Alzheimer's disease. Enhancements to the LDA allow stepwise inclusion (or removal) of variables to optimize the discriminant power of the model. The result of the LDA is therefore a cluster of AFs or APIs which can be used for diagnosis, treatment or development of pharmaceutical products. Other enhanced variations of LDA, such as Flexible Discriminant Analysis permit the use of non-linear combinations of variables to discriminate a disease state from a state in which there is no disease. The results of the discriminant analysis can be verified by post-hoc tests and also by repeating the analysis using alternative techniques such as classification trees.

A further category of AFs or APIs can be identified by qualitative measures by comparing the percentage feature presence of an AF or API of one group of samples (e.g., samples from diseased subjects) with the percentage feature presence of an AF or API in another group of samples (e.g., samples from control subjects). The “percentage feature presence” of an AF or API is the percentage of samples in a group of samples in which the AF or API is detectable by the detection method of choice. For example, if an AF is detectable in 95 percent of samples from diseased subjects, the percentage feature presence of that AF in that sample group is 95 percent. If only 5 percent of samples from non-diseased subjects have detectable levels of the same AF, detection of that AF in the sample of a subject would suggest that it is likely that the subject has Alzheimers disease.

Use in Clinical Studies

The diagnostic methods and compositions of the present invention can assist in monitoring a clinical study, e.g. to evaluate therapies for Alzheimer's disease. In one embodiment, chemical compounds are tested for their ability to restore AF or API levels in a subject having Alzheimer's disease to levels found in subjects free from Alzheimer's disease or, in a treated subject (e.g. after treatment with a cholinesterase inhibitor), to preserve AF or API levels at or near levels seen in subjects free from Alzheimer's disease. The levels of one or more AFs or APIs can be assayed.

In another embodiment, the methods and compositions of the present invention are used to screen individuals for entry into a clinical study to identify individuals having Alzheimer's disease; individuals already having Alzheimer's disease can then be excluded from the study or can be placed in a separate cohort for treatment or analysis. If desired, the candidates can concurrently be screened to identify individuals with Lewy Body disease and/or senile dementia or other known measured of Alzheimer's disease; procedures for these screens are well known-in the-art (Harding and Halliday, 1998, Neuropathol. Appl. Neurobiol. 24:195-201).

Purification of APIs

In particular aspects, the invention provides isolated mammalian APIs, preferably human APIs, and fragments thereof which comprise an antigenic determinant (i.e., can be recognized by an antibody) or which are otherwise functionally active, as well as nucleic acid sequences encoding the foregoing. “Functionally active” as used herein refers to material displaying one or more functional activities associated with a full-length (wild-type) API, e.g., binding to an API substrate or API binding partner, antigenicity (binding to an anti-API antibody), immunogenicity, enzymatic activity and the like.

In specific embodiments, the invention provides fragments of an API comprising at least 5 amino acids, at least 10 amino acids, at least 50 amino acids, or at least 75 amino acids. Fragments lacking some or all of the regions of an API are also provided, as are proteins (e.g., fusion proteins) comprising such fragments. Nucleic acids encoding the foregoing are provided.

Once a recombinant nucleic acid which encodes the API, a portion of the API, or a precursor of the API is identified, the gene product can be analyzed. This can be achieved by assays based on the physical or functional properties of the given product, including, for example, radioactive labeling of the product followed by analysis by gel electrophoresis, immunoassay, etc.

The APIs identified herein can be isolated and purified by standard methods including chromatography (e.g., ion exchange, affinity, and sizing column chromatography), centrifugation, differential solubility, or by any other standard technique for the purification of proteins.

Alternatively, once a recombinant nucleic acid that encodes the API is identified, the entire amino acid sequence of the API can be deduced from the nucleotide sequence of the gene coding region contained in the recombinant nucleic acid. As a result, the protein can be synthesized by standard chemical methods known in the art (e.g., see Hunkapiller et al., 1984, Nature 310:105-111).

In another alternative embodiment, native APIs can be purified from natural sources, by standard methods such as those described above (e.g., immunoaffinity purification).

In a preferred embodiment, APIs are isolated by the Preferred Technology described supra. For preparative-scale runs, a narrow-range “zoom gel” having a pH range of 2 pH units or less is preferred for the isoelectric step, according to the method described in Westermeier, 1993, Electrophoresis in Practice (VCH, Weinheim, Germany), pp. 197-209 (which is incorporated herein by reference in its entirety); this modification permits a larger quantity of a target protein to be loaded onto the gel, and thereby increases the quantity of isolated API that can be recovered from the gel. When used in this way for preparative-scale runs, the Preferred Technology typically provides up to 100 ng, and can provide up to 1000 ng, of an isolated API in a single run. Those of skill in the art will appreciate that a zoom gel can be used in any separation strategy which employs gel isoelectric focusing.

The invention thus provides an isolated API, an isolated API-related polypeptide, and an isolated derivative or fragment of an API or an API-related polypeptide; any of the foregoing can be produced by recombinant DNA techniques or by chemical synthetic methods.

Isolation of DNA Encoding an API

Particular embodiments for the cloning of a gene encoding an API, are presented below by way of example and not of limitation.

The nucleotide sequences of the present invention, including DNA and RNA, and comprising a sequence encoding an API or a fragment thereof, or an API-related polypeptide, may be synthesized using methods known in the art, such as using conventional chemical approaches or polymerase chain reaction (PCR) amplification. The nucleotide sequences of the present invention also permit the identification and cloning of the gene encoding an API homolog or API ortholog including, for example, by screening cDNA libraries, genomic libraries or expression libraries.

For example, to clone a gene encoding an API by PCR techniques, anchored degenerate oligonucleotides (or a set of most likely oligonucleotides) can be designed for all API peptide fragments identified as part of the same protein. PCR reactions under a variety of conditions can be performed with relevant cDNA and genomic DNAs (e.g., from brain tissue or from cells of the immune system) from one or more species. Also vectorette reactions can be performed on any available cDNA and genomic DNA using the oligonucleotides (which preferably are nested) as above. Vectorette PCR is a method that enables the amplification of specific DNA fragments in situations where the sequence of only one primer is known. Thus, it extends the application of PCR to stretches of DNA where the sequence information is only available at one end. (Arnold C, 1991, PCR Methods Appl. 1(1):39-42; Dyer K D, Biotechniques, 1995, 19(4):550-2). Vectorette PCR may be performed with probes that are, for example, anchored degenerate oligonucleotides (or most likely oligonucleotides) coding for API peptide fragments, using as a template a genomic library or cDNA library pools.

Anchored degenerate oligonucleotides (and most likely oligonucleotides) can be designed for all API peptide fragments. These oligonucleotides may be labelled and hybridized to filters containing cDNA and genomic DNA libraries. Oligonucleotides to different peptides from the same protein will often identify the same members of the library. The cDNA and genomic DNA libraries may be obtained from any suitable or desired mammalian species, for example from humans.

Nucleotide sequences comprising a nucleotide sequence encoding an API or API fragment of the present invention are useful, for example, for their ability to hybridize selectively with complementary stretches of genes encoding other proteins. Depending on the application, a variety of hybridization conditions may be employed to obtain nucleotide sequences at least about 30%, 35%, 40%, 45%, 50%, 55%, 60%, 65%, 70%, 75%, 80%, 85%, 90%, 95% or 99% identical, or 100% identical, to the sequence of a nucleotide encoding an API.

For a high degree of selectivity, relatively stringent conditions are used to form the duplexes, such as low salt or high temperature conditions. As used herein, “highly stringent conditions” means hybridization to filter-bound DNA in 0.5 M NaHPO4, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. 1, Green Publishing Associates, Inc., and John Wiley & Sons, Inc., New York, at p. 2.10.3; incorporated herein by reference in its entirety.) For some applications, less stringent conditions for duplex formation are required. As used herein “moderately stringent conditions” means washing in 0.2×SSC/0.1% SDS at 42° C. (Ausubel et al., 1989, supra). Hybridization conditions can also be rendered more stringent by the addition of increasing amounts of formamide, to destabilize the hybrid duplex. Thus, particular hybridization conditions can be readily manipulated, and will generally be chosen depending on the desired results. In general, convenient hybridization temperatures in the presence of 50% formamide are: 42° C. for a probe which is 95 to 100% identical to the fragment of a gene encoding an API, 37° C. for 90 to 95% identity and 32° C. for 70 to 90% identity.

In the preparation of genomic libraries, DNA fragments are generated, some of which will encode parts or the whole of an API. Any suitable method for preparing DNA fragments may be used in the present invention. For example, the DNA may be cleaved at specific sites using various restriction enzymes. Alternatively, one may use DNAse in the presence of manganese to fragment the DNA, or the DNA can be physically sheared, as for example, by sonication. The DNA fragments can then be separated according to size by standard techniques, including but not limited to agarose and polyacrylamide gel electrophoresis, column chromatography and sucrose radient centrifugation. The DNA fragments can then be inserted into suitable vectors, including but not limited to plasmids, cosmids, bacteriophages lambda or T4, and yeast artificial chromosome (YAC). (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II; Ausubel F. M. et al., eds., 1989, Current Protocols in Molecular Biology, Vol. 1, Green Publishing Associates, Inc., and John Wiley & sons, Inc., New York). The genomic library may be screened by nucleic acid hybridization to labeled probe (Benton and Davis, 1977, Science 196:180; Grunstein and Hogness, 1975, Proc. Natl. Acad. Sci. U.S.A. 72:3961).

Based on the present description, the genomic libraries may be screened-with labeled degenerate oligonucleotide probes corresponding to the amino acid sequence of any peptide of the API using optimal approaches well known in the art. Any probe used is at least 10 nucleotides, at least 15 nucleotides, at least 20 nucleotides, at least 25 nucleotides, at least 30 nucleotides, at least 40 nucleotides, at least 50 nucleotides, at least 60 nucleotides, at least 70 nucleotides, at least 80 nucleotides, or at least 100 nucleotides. Preferably a probe is 10 nucleotides or longer, and more preferably 15 nucleotides or longer.

In Tables IV and V above, some APIs disclosed herein correspond to isoforms of previously identified proteins encoded by genes whose sequences are publicly known. To screen such a gene, any probe may be used that is complementary to the gene or its complement; preferably the probe is 10 nucleotides or longer, more preferably 15 nucleotides or longer; The SWISS-PROT and trEMBL databases (held by the Swiss Institute of Bioinformatics (SIB) and the European Bioinformatics Institute (EBI) which are available at http://www.expasy.ch/) and the GenBank database (held by the National Institute of Health (NIH) which is available at http://www.ncbi.nlm.nih.gov/) provide protein sequences comprising the amino acid sequences listed for the APIs in Tables IV and V under the following accession numbers and each sequence is incorporated herein by reference:

TABLE VIII
Nucleotide sequences encoding APIs, API Related Proteins, or ERPIs

| | | Accession Numbers of |
|---|---|---|
| AF# | API# | Identified Sequences |
| AF-1 | API-47 | O15179 |
| AF-1 | API-242 | AAF03259 |
| AF-2 | API-1 | P10645 |
| AF-3 | API-48 | Q9UBQ6 |
| AF-5 | API-49 | P19021 |
| AF-6 | API-2 | P47868 |
| AF-8 | API-194 | AAB60937 |
| AF-9 | API-3 | O15179 |
| AF-10 | API-50 | P02649 |
| AF-10 | API-51 | P55290 |
| AF-13 | API-4 | P01023 |
| AF-14 | API-52 | P01011 |
| AF-14 | API-243 | P04004 |
| AF-15 | API-53 | P10909 |
| AF-15 | API-244 | AAC50896 |
| AF-16 | API-54 | P19021 |
| AF-17 | API-5 | O43505 |
| AF-18 | API-55 | P09104 |
| AF-18 | API-245 | P51693 |
| AF-21 | API-6 | O15179 |
| AF-22 | API-56 | O94985 |
| AF-22 | API-57 | AAD05198 |
| AF-23 | API-7 | P05090 |
| AF-23 | API-8 | P05060 |
| AF-24 | API-9 | P19021 |
| AF-25 | API-10 | P10909 |
| AF-26 | API-14 | P41222 |
| AF-27 | API-15 | P20758 |
| AF-27 | API-58 | O15179 |
| AF-28 | API-16 | Q04857 |
| AF-28 | API-59 | P00450 |
| AF-29 | API-196 | P01034 |
| AF-30 | API-17 | P36955 |
| AF-31 | API-60 | P06396 |
| AF-32 | API-18 | O43505 |
| AF-34 | API-61 | Q14800 |
| AF-35 | API-62 | P02649 |
| AF-37 | API-19 | P40925 |
| AF-38 | API-63 | Q92876 |
| AF-39 | API-64 | P01028 |
| AF-39 | API-65 | Q9UBQ6 |
| AF-40 | API-20 | P17174 |
| AF-41 | API-22 | P18065 |
| AF-42 | API-66 | P02753 |
| AF-43 | API-67 | P02649 |
| AF-43 | API-68 | P01034 |
| AF-44 | API-69 | P00751 |
| AF-44 | API-70 | P10643 |
| AF-45 | API-23 | P36955 |
| AF-46 | API-24 | P05067 |
| AF-46 | API-197 | P13645 |
| AF-46 | API-198 | P13647 |
| AF-47 | API-25 | O43505 |
| AF-48 | API-71 | Q99435 |
| AF-49 | API-26 | Q92876 |
| AF-49 | API-27 | P41222 |
| AF-50 | API-72 | P01871 |
| AF-50 | API-73 | P00748 |
| AF-50 | API-199 | P04196 |
| AF-50 | API-200 | AAC34741 |
| AF-51 | API-28 | P10909 |
| AF-51 | API-30 | P07195 |
| AF-52 | API-74 | P01028 |
| AF-53 | API-33 | P02768 |
| AF-54 | API-221 | P02649 |
| AF-55 | API-34 | P01034 |
| AF-56 | API-75 | P02675 |
| AF-56 | API-246 | P06396 |
| AF-57 | API-35 | P35527 |
| AF-57 | API-76 | P01024 |
| AF-57 | API-222 | P13645 |
| AF-58 | API-77 | P02649 |
| AF-59 | API-36 | P36955 |
| AF-60 | API-37 | P02649 |
| AF-61 | API-78 | P01024 |
| AF-62 | API-38 | P01028 |
| AF-63 | API-79 | P01024 |
| AF-64 | API-80 | P01024 |
| AF-65 | API-81 | P00751 |
| AF-65 | API-223 | P01028 |
| AF-66 | API-82 | P01024 |
| AF-66 | API-83 | P01034 |
| AF-67 | API-39 | P02766 |
| AF-68 | API-84 | P04406 |
| AF-68 | API-85 | P04279 |
| AF-69 | API-40 | P06727 |
| AF-69 | API-247 | CAB89302 |
| AF-70 | API-41 | P36222 |
| AF-70 | API-224 | 229552 (gb) |
| AF-71 | API-42 | P01024 |
| AF-72 | API-43 | P06727 |
| AF-73 | API-44 | P02766 |
| AF-74 | API-45 | P02790 |
| AF-74 | API-248 | P01024 |
| AF-75 | API-46 | P10909 |
| AF-75 | API-225 | P02649 |
| AF-76 | API-86 | P19021 |
| AF-79 | API-201 | P01023 |
| AF-81 | API-88 | AAA52900 |
| AF-81 | API-202 | AAC48775 |
| AF-82 | API-89 | P09571 |
| AF-83 | API-90 | P09571 |
| AF-84 | API-91 | P09571 |
| AF-85 | API-92 | P01024 |
| AF-85 | API-93 | P09571 |
| AF-87 | API-95 | P08835 |
| AF-89 | API-97 | P01024 |
| AF-90 | API-98 | P07358 |
| AF-91 | API-99 | S64635 |
| AF-100 | API-101 | P08835 |
| AF-103 | API-102 | Q03591 |
| AF-104 | API-103 | P06866 |
| AF-105 | API-104 | P02766 |
| AF-107 | API-107 | Q16270 |
| AF-107 | API-210 | BAA25513 |
| AF-108 | API-108 | O88812 |
| AF-117 | API-113 | P02649 |
| AF-119 | API-114 | P02023 |
| AF-121 | API-116 | P04469 |
| AF-123 | API-118 | P02671 |
| AF-124 | API-119 | P02671 |
| AF-125 | API-120 | Q12805 |
| AF-126 | API-121 | O43532 |
| AF-126 | API-122 | AAF02676 |
| AF-127 | API-123 | P01019 |
| AF-128 | API-124 | P01028 |
| AF-129 | API-125 | P02774 |
| AF-129 | API-126 | P01019 |
| AF-130 | API-127 | P36955 |
| AF-130 | API-128 | O43505 |
| AF-132 | API-130 | P01028 |
| AF-133 | API-131 | P06727 |
| AF-134 | API-132 | P06727 |
| AF-137 | API-134 | P10909 |
| AF-137 | API-135 | P05156 |
| AF-137 | API-232 | Q9Y6R4 |
| AF-137 | API-233 | P02649 |
| AF-137 | API-234 | P33176 |
| AF-139 | API-136 | P10909 |
| AF-139 | API-137 | P02649 |
| AF-140 | API-138 | P01028 |
| AF-141 | API-139 | P09871 |
| AF-142 | API-140 | P01024 |
| AF-142 | API-141 | Q92876 |
| AF-143 | API-142 | 751423A |
| AF-144 | API-143 | P16035 |
| AF-149 | API-214 | AAB60937 |
| AF-150 | API-144 | Q02246 |
| AF-151 | API-145 | O15179 |
| AF-152 | API-146 | P43652 |
| AF-152 | API-147 | P51693 |
| AF-152 | API-148 | P00734 |
| AF-153 | API-149 | P01028 |
| AF-154 | API-150 | P08697 |
| AF-154 | API-151 | P02748 |
| AF-154 | API-152 | P01877 |
| AF-155 | API-215 | P01034 |
| AF-156 | API-153 | AF177396 |
| AF-157 | API-155 | O43505 |
| AF-159 | API-158 | P36955 |
| AF-159 | API-159 | O43505 |
| AF-159 | API-160 | P07339 |
| AF-161 | API-161 | P06727 |
| AF-161 | API-162 | P36955 |
| AF-161 | API-163 | P02570 |
| AF-163 | API-165 | P05090 |
| AF-163 | API-166 | P05060 |
| AF-164 | API-167 | P10909 |
| AF-165 | API-168 | P01028 |
| AF-166 | API-169 | P01028 |
| AF-167 | API-170 | P10909 |
| AF-167 | API-171 | AAD02505 |
| AF-168 | API-237 | P13645 |
| AF-168 | API-172 | P01028 |
| AF-169 | API-173 | P01028 |
| AF-170 | API-174 | P10909 |
| AF-170 | API-175 | P02766 |
| AF-170 | API-176 | AAD02505 |
| AF-171 | API-177 | P41222 |
| AF-171 | API-178 | P02766 |
| AF-172 | API-179 | P24592 |
| AF-172 | API-180 | AAD51475 |
| AF-173 | API-181 | P01024 |
| AF-174 | API-182 | P29361 |
| AF-175 | API-183 | P41222 |
| AF-176 | API-184 | P41222 |
| AF-178 | API-185 | P47971 |
| AF-178 | API-217 | P41222 |
| AF-178 | API-219 | Q29562 |
| AF-179 | API-186 | P02753 |
| AF-180 | API-220 | P01028 |
| AF-181 | API-187 | P00441 |
| AF-182 | API-188 | P02766 |
| AF-183 | API-189 | P01034 |
| AF-184 | API-190 | P36955 |
| AF-185 | API-191 | P00737 |
| AF-185 | API-192 | Q12805 |
| AF-186 | API-238 | P05090 |
| AF-187 | API-239 | O15179 |
| AF-190 | API-240 | NP_055108 (gb) |
| AF-192 | API-241 | P19021 |
| ERF-2 | ERPI-1 | P36955 |

When no nucleotide sequence is known that encodes a protein comprising an amino acid sequence of a given API, degenerate probes can be used for screening. In Table IX, a degenerate set of probes is provided for API-111 and API-112. The partial amino acid sequences listed in Table IX were derived from manual interpretation of the tandem mass spectra of tryptic digest peptides of the API. In the method of tandem mass spectroscopy used for sequencing peptides in the present invention, the following pairs of amino acids could not be distinguished from each other: leucine and isoleucine; and, under certain circumstances, phenylalanine and oxidized methionine. As used herein, an amino acid sequence “as determined by mass spectrometry” refers to the set of amino acid sequences containing at the indicated positions, one or other member of the designated pairs of amino acids. For example, the amino acid sequence P[L/I]A indicates the amino acid sequences PLA and PIA. As will be obvious to one of skill in the art, a sequence containing n designated pairs indicates 2n amino acid sequences. In Table IX, each possible amino acid sequence is listed for each sequence determined by mass spectroscopy, and preferred and fully degenerate sets of probes for each possible amino acid sequence are provided.

TABLE IX
Amino Acid Sequences and Probes for APIs

| | | Amino Acid Sequences of Tryptic Digest Peptides | | | | | |
|---|---|---|---|---|---|---|---|
| | | as Determined by Mass Spectrometry | | | | | |
| | | Mass of | | | | | |
| | | singly | | | | | |
| | | protonated | | | | | |
| | | peptide | Partial | N-terminal | C-terminal | Preferred | Degenerate |
| AF# | API# | (Da)* | sequence | Mass (Da)* | Mass (Da)* | Probes | Probes |
| AF-114 | API-111 | 1097.57 | HQV | 0 | 733.50 | CACCAGGTG | CAYCARGTN |
| AF-114 | API-112 | 1547.74 | PGLGM | 0 | 1076.63 | CCCGGCCT | CCNGGNYT |
| | | | | | | GGGCATG | NGGNATG |
| AF-114 | API-112 | 1547.74 | PGLGF | 0 | 1076.63 | CCCGGCCT | CCNGGNYT |
| | | | | | | GGGCTTC | NGGNTTY |
| AF-114 | API-112 | 1547.74 | PGIGM | 0 | 1076.63 | CCCGGCAT | CCNGGNAT |
| | | | | | | CGGCATG | HGGNATG |
| AF-114 | API-112 | 1547.74 | PGIGF | 0 | 1076.63 | CCCGGCAT | CCNGGNAT |
| | | | | | | CGGCTTC | HGGNTTY |
| AF-114 | API-112 | 1547.74 | GPLGM | 0 | 1076.63 | GGCCCCCT | GGNCCNYT |
| | | | | | | GGGCATG | NGGNATG |
| AF-114 | API-112 | 1547.74 | GPLGF | 0 | 1076.63 | GGCCCCCT | GGNCCNYT |
| | | | | | | GGGCTTC | NGGNTTY |
| AF-114 | API-112 | 1547.74 | GPIGM | 0 | 1076.63 | GGCCCCAT | GGNCCNAT |
| | | | | | | CGGCATG | HGGNATG |
| AF-114 | API-112 | 1547.74 | GPIGF | 0 | 1076.63 | GGCCCCAT | GGNCCNAT |
| | | | | | | CGGCTTC | HGGNTTY |

*The masses determined by mass spectrometry have an error of mass measurement of 100 parts-per-million (ppm) or less. For a given measured mass, M, having an error of mass measurement of z ppm, the error of mass measurement can be calculated as (M × z ÷ 1000000).

As used herein, the “mass of the singly protonated peptide” is the mass of the singly protonated tryptic digest peptide measured by mass spectrometry (having an error of measurement of approximately 100 parts-per-million or less) and corresponds to the total mass of the constituent amino acid residues of the peptide with the addition of a water molecule (H₂O) and a single proton (H⁺). As used herein, an “amino acid residue” refers to an amino acid residue of the general structure: —NH—CHR—CO— and which have the following symbols, elemental compositions and monoisotopic masses:

Amino acid residue elemental compositions and monoisotopic masses

| | | Elemental | Monoisotopic |
|---|---|---|---|
| Amino Acid | Symbol | Composition | mass (Da) |
| Alanine | A | C₃H₅NO | 71.037114 |
| Arginine | R | C₆H₁₂N₄O | 156.10111 |
| Asparagine | N | C₄H₆N₂O₂ | 114.042927 |
| Aspartic Acid | D | C₄H₅NO₃ | 115.026943 |
| Carboxyamido | C | C₅H₈N₂O₂S | 160.03065 |
| Cysteine¹ | | | |
| Glutamic Acid | E | C₅H₇NO₃ | 129.042593 |
| Glutamine | Q | C₃H₈N₂O₂ | 128.058577 |
| Glycine | G | C₂H₃NO | 57.021464 |
| Histadine | H | C₆H₇N₃O | 137.058912 |
| Isoleucine | I | C₆H₁₁NO | 113.084064 |
| Leucine | L | C₆H₁₁NO | 113.084064 |
| Lysine | K | C₆H₁₂N₂O | 128.094963 |
| Methionine | M | C₅H₉NOS | 131.040485 |
| Oxidised | M* | C₅H₉NO₂S | 147.035340 |
| Methionine | | | |
| Phenylalanine | F | C₉H₉NO | 147.068414 |
| Proline | P | C₅H₇NO | 97.052764 |
| Serine | S | C₃H₅NO₂ | 87.032028 |
| Threonine | T | C₄H₇NO₂ | 101.047678 |
| Tryptophan | W | C₁₁H₁₀ N₂O | 186.079313 |
| Tyrosine | Y | C₉H₉NO₂ | 163.063328 |
| Valine | V | C₅H₉NO | 99.068414 |

¹All Cysteines are modified to the carboxyamino derivative during our production process.

As used herein “tryptic digest peptides” are peptides produced through treatment of the protein with the enzyme trypsin. Trypsin cleaves specifically at the carboxyl side of lysine (Lys) and arginine (Arg) residues, so that the tryptic digest peptides generated should have a Lys or Arg as the C-terminal amino acid, unless the peptide fragment was obtained from the C-terminal of the protein. Similarly, the amino acid directly preceding the N-terminal amino acid of the tryptic digest peptides should also be a Lys or Arg, unless the peptide was obtained from the N-terminal of the protein. The mass of a tryptic digest peptide corresponds to the total mass of the constituent amino acid residues of the peptide with the addition of a water molecule (H₂O). As used herein, the “partial sequence” is an amino acid-sequence within the tryptic digest peptide determined from the interpretation of the tandem mass spectrum of the peptide. As used herein, the “N-terminal mass” is the mass measured by mass spectrometry (having an error of measurement of approximately 100 parts-per-million or less) of the portion of the tryptic digest peptide extending from the start of the partial sequence to the N-terminus of the peptide. This is a neutral mass corresponding to the total mass of the constituent amino acid residues extending from the partial sequence to the N-terminus of the peptide. As used herein, the “C-terminal mass” is the mass measured by mass spectrometry (having an error of measurement of approximately 100 parts-per-million or less) of the portion of the tryptic digest peptide extending from the end of the partial sequence to the- C-terminus of the peptide. This mass corresponds to the total mass of the constituent amino acid residues extending from the end of the partial sequence to the C-terminus of the peptide with the addition of a water molecular (H₂O), and a single proton (H⁺). In Table IX, supra, the preferred and degenerate sets of probes are described using GCG Nucleotide Ambiguity Codes as employed in GCG SeqWeb™ sequence analysis software (SeqWeb™ version 1.1, part of Wisconsin Package Version 10, Genetics Computer Group, Inc.). These Nucleotide Ambiguity Codes have the following meaning:

| GCG Code | Meaning |
|---|---|
| A | A |
| C | C |
| G | G |
| T | T |
| U | T |
| M | A or C |
| R | A or G |
| W | A or T |
| S | C or G |
| Y | C or T |
| K | G or T |
| V | A or C or G |
| H | A or C or T |
| D | A or G or T |
| B | C or G or T |
| X | G or A or T or C |
| N | G or A or T |
| | or C |

GCG uses the letter codes for amino acid codes and nucleotide ambiguity proposed by UPAC-IUB. These codes are compatible with the codes used by the EMBL, GenBank, and PIR databases. See IUPAC, Commission on Nomenclature of Organic Chemistry. A Guide to IUPAC Nomenclature of Organic Compounds (Recommendations 1993), Blackwell Scientific publications, 1993.

When a library is screened, clones with insert DNA encoding the API of interest or a fragment thereof will hybridize to one or more members of the corresponding set of degenerate oligonucleotide probes (or their complement). Hybridization of such oligonucleotide probes to genomic libraries is carried out using methods known in the art. For example, hybridization with one of the above-mentioned degenerate sets of oligonucleotide probes, or their complement (or with any member of such a set, or its complement) can be performed under highly stringent or moderately stringent conditions as defined above, or can be carried out in 2×SSC, 1.0% SDS at 50° C. and washed using the washing conditions described supra for highly stringent or moderately stringent hybridization.

In yet another aspect of the invention, clones containing nucleotide sequences encoding the entire API, a fragment of an API, an API-related polypeptide, or a fragment of an API-related polypeptide or any of the foregoing may also be obtained by screening expression libraries. For example, DNA from the relevant source is isolated and random fragments are prepared and ligated into an expression vector (e.g., a bacteriophage, plasmid, phagemid or cosmid) such that the inserted sequence in the vector is capable of being expressed by the host cell into which the vector is then introduced. Various screening assays can then be used to select for the expressed API or API-related polypeptides. In one embodiment, the various anti-API antibodies of the invention can be used to identify the desired clones using methods known in the art. See, for example, Harlow and Lane, 1988, Antibodies: A Laboratory Manual, Cold Spring Harbor Laboratory Press, Cold Spring Harbor, N.Y., Appendix IV. Colonies-or plaques from the library are brought into contact with the antibodies to identify those clones that bind antibody.

In an embodiment, colonies or plaques containing DNA that encodes an API, a fragment of an API, an API-related polypeptide, or a fragment of an API-related polypeptide can be detected using DYNA Beads according to Olsvick et al., 29th ICAAC, Houston, Tex. 1989, incorporated herein by reference. Anti-API antibodies are crosslinked to tosylated DYNA Beads M280, and these antibody-containing beads are then contacted with colonies or plaques expressing recombinant polypeptides. Colonies or plaques expressing an API or API-related polypeptide are identified as any of those that bind the beads.

Alternatively, the anti-API antibodies can be nonspecifically immobilized to a suitable support, such as silica or Celite® resin. This material is then used-to adsorb to bacterial colonies expressing the API protein or API-related polypeptide as described herein.

In another aspect, PCR amplification may be used to isolate from genomic DNA a substantially pure DNA (i.e., a DNA substantially free of contaminating nucleic acids) encoding the entire API or a part thereof. Preferably such a DNA is at least 95% pure, more preferably at least 99% pure. Oligonucleotide sequences, degenerate or otherwise, that correspond to peptide sequences of APIs disclosed herein can be used as primers.

PCR can be carried out, e.g., by use of a Perkin-Elmer Cetus thermal cycler and Taq polymerase (Gene Amp® or AmpliTaq DNA polymerase). One can choose to synthesize several different degenerate primers, for use in the PCR reactions. It is also possible to vary the stringency of hybridization conditions used in priming the PCR reactions, to allow for greater or lesser degrees of nucleotide sequence similarity between the degenerate primers and the corresponding sequences in the DNA. After successful amplification of a segment of the sequence encoding an API, that segment may be molecularly cloned and sequenced, and utilized as a probe to isolate a complete genomic clone. This, in turn, will permit the determination of the gene's complete nucleotide sequence, the analysis of its expression, and the production of its protein product for functional analysis, as described infra.

The gene encoding an API can also be identified by mRNA selection by nucleic acid hybridization followed by in vitro translation. In this procedure, fragments are used to isolate complementary mRNAs by hybridization. Such DNA fragments may represent available, purified DNA encoding an API of another species (e.g., mouse, human). Immunoprecipitation analysis or functional assays (e.g., aggregation ability in vitro; binding to receptor) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies that specifically recognize an API. A radiolabelled cDNA encoding an API can be synthesized using the selected mRNA (from the adsorbed polysomes) as a template. The radiolabelled mRNA or cDNA may then be used as a probe to identify the DNA fragments encoding an API from among other genomic DNA fragments.

Alternatives to isolating genomic DNA encoding an API include, but are not limited to, chemically synthesizing the gene sequence itself from a known sequence or making cDNA to the mRNA which encodes the API. For example, RNA for cDNA cloning of the gene encoding an API can be isolated from cells which express the API. Those skilled in the art will understand from the present description that other methods may be used and are within the scope of the invention.

Any suitable eukaryotic cell can serve as the nucleic acid source for the molecular cloning of the gene encoding an API. The nucleic acid sequences encoding the API can be isolated from vertebrate, mammalian, primate, human, porcine, bovine, feline, avian, equine, canine or murine sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell. (See, e.g., Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II.) Clones derived from genomic DNA may contain regulatory and intron DNA regions in addition to coding regions; clones derived from cDNA will contain only exon sequences.

The identified and isolated gene or cDNA can then be inserted into any suitable cloning vector. A large number of vector-host systems known in the art may be used. As those skilled in the art will appreciate, the vector system chosen should be compatible with the host cell used. Such vectors include, but are not limited to, bacteriophages such as lambda derivatives, plasmids such as PBR322 or pUC plasmid derivatives or the Bluescript vector (Stratagene) or modified viruses such as adenoviruses, adeno-associated viruses or retroviruses. The insertion into a cloning vector can be accomplished, for example, by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and the gene encoding an API may be modified by homopolymeric tailing. Recombinant molecules can be introduced into host cells via transformation, transfection, infection, electroporation, etc., so that many copies of the gene sequence are generated.

In specific embodiments, transformation of host cells with recombinant DNA molecules that incorporate the isolated gene encoding the API, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The nucleotide sequences of the present invention include nucleotide sequences encoding amino acid sequences with substantially the same amino acid sequences as native APIs, nucleotide sequences encoding amino acid sequences with functionally equivalent amino acids, nucleotide sequences encoding APIs, a fragments of APIs, API-related polypeptides, or fragments of API-related polypeptides.

In a specific embodiment, an isolated nucleic acid molecule encoding an API-related polypeptide can be created by introducing one or more nucleotide substitutions, additions or deletions into the nucleotide sequence of an API such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Expression of DNA Encoding APIs

The nucleotide sequence coding for an API, an API analog, an API-related peptide, or a fragment or other derivative of any of the foregoing, can be inserted into an appropriate expression vector, ie., a vector which contains the necessary elements for the transcription and translation of the inserted protein-coding sequence. The necessary transcriptional and translational signals can also be supplied by the native gene encoding the API or its flanking regions, or the native gene encoding the API-related polypeptide or its flanking regions. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In specific embodiments, a nucleotide sequence encoding a human gene (or a nucleotide sequence encoding a functionally active portion of a human API) is expressed. In yet another embodiment, a fragment of an API comprising a domain of the API is expressed.

Any of the methods previously described for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of nucleic acid sequence encoding an API or fragment thereof may be regulated by a second nucleic acid sequence so that the API or fragment is expressed in a host transformed with the recombinant DNA molecule. For example, expression of an API may be controlled by any promoter or enhancer element known in the art. Promoters which may be used to control the expression of the gene encoding an API or an API-related polypeptide include, but are not limited to, the SV40 early promoter region (Bernoist and Chambon, 1981, Nature 290:304-3 10), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the β-lactamase promoter (Villa-Kamaroff, et al;, 1978, Proc. Natl. Acad. Sci. U.S.A. 75:3727-3731), or the tac promoter (DeBoer, et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus 35S RNA promoter (Gardner, et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Corn. 253:818-823); glial fibrillary acidic protein (GFAP) promoter which is active in astrocytes (Gomes et al., 1999, Braz J Med Biol Res 32(5):619-631; Morelli et al., 1999, Gen. Virol. 80:571-83) and gonadotropic releasing hormone gene control region which is active in the hypothalamus (Mason et al., 1986, Science 234:1372-1378).

In a specific embodiment, a vector is used that comprises a promoter operably linked to an API-encoding nucleic acid, one or more origins of replication, and, optionally, one or more selectable markers (e.g., an antibiotic resistance gene).

In a specific embodiment, an expression construct is made by subcloning an API or an API-related polypeptide coding sequence into the EcoRI restriction site of each of the three pGEX vectors (Glutathione S-Transferase expression vectors; Smith and Johnson, 1988, Gene 7:31-40). This allows for the expression of the API product or API-related polypeptide from the subclone in the correct reading frame.

In mammalian host cells, a number of viral-based expression systems may be utilized. In cases where an adenovirus is used as an expression vector, the API coding sequence or API-related polypeptide coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, 1984, Proc. Natl. Acad. Sci. USA 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding an API or an API-related polypeptide can be identified, for example, by three general approaches: (a) nucleic acid hybridization, (b) presence or absence of “marker” gene functions, and (c) expression of inserted sequences. In the first approach, the presence of a gene encoding an API inserted in an expression vector can be detected by nucleic acid hybridization using probes comprising sequences that are homologous to an inserted gene encoding an API. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a gene encoding an API in the vector. For example, if the gene encoding the API is inserted within the marker gene sequence of the vector, recombinants containing the gene encoding the API insert can be identified by the absence of the marker gene function. In the third approach, recombinant expression vectors can be identified by assaying the gene product (i.e., API) expressed by the recombinant. Such assays can be based, for example, on the physical or functional properties of the API in in vitro assay systems, e.g., binding with anti-API antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered API or API-related polypeptide may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include but are not limited to CHO, VERO, BHK, Hela, COS, MDCK, 293, 3T3, WI38, and in particular, neuronal cell lines such as, for example, SK-N-AS, SK-N-FI, SK-N-DZ human neuroblastomas (Sugimoto et al., 1984, J. Natl. Cancer Inst. 73: 51-57), SK-N-SH human neuroblastoma (Biochim. Biophys. Acta, 1982, 704: 450-460), Daoy human cerebellar medulloblastoma (He et al., 1992, Cancer Res. 52: 1144-1148) DBTRG-05MG glioblastoma cells (Kruse et al., 1992, In vitro Cell. Dev. Biol. 28A: 609-614), IMR-32 human neuroblastoma (Cancer Res., 1970, 30: 2110-2118), 1321N1 human astrocytoma (Proc. Natl Acad. Sci. USA ,1977, 74: 4816), MOG-G-CCM human astrocytoma (Br. J. Cancer, 1984, 49: 269), U87MG human glioblastoma-astrocytoma (Acta Pathol. Microbiol. Scand., 1968, 74: 465-486), A172 human glioblastoma (Olopade et al., 1992, Cancer Res. 52: 2523-2529), C6 rat glioma cells (Benda et al., 1968, Science 161: 370-371), Neuro-2a mouse neuroblastoma (Proc. Natl. Acad. Sci. USA, 1970, 65: 129-136), NB41A3 mouse neuroblastoma (Proc. Natl. Acad. Sci. USA, 1962, 48: 1184-1190), SCP sheep choroid plexus (Bolin et al., 1994, J. Virol. Methods 48: 211-221), G355-5, PG-4 Cat normal astrocyte (Haapala et al., 1985, J. Virol. 53: 827-833), Mpf ferret brain (Trowbridge et al., 1982, In vitro 18: 952-960), and normal cell lines such as, for example, CTX TNA2 rat normal cortex brain (Radany et al., 1992, Proc. Natl. Acad. Sci. USA 89:-6467-6471) such as, for example, CRL7030 and Hs578Bst. Furthermore, different vector/host expression systems may effect processing reactions to different extents.

For long-term, high-yield production of recombi-nant proteins, stable expression is preferred. For example, cell lines which stably express the differentially expressed or pathway gene protein may be engineered. Rather than using expression vectors which cortain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the differentially expressed or pathway gene protein. Such engineered cell lines may be particularly useful in screening and evaluation of compounds that affect the endogenous activity of the differentially expressed or pathway gene protein.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt- cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

In other embodiments, the API, fragment, analog, or derivative may be expressed as a fusion, or chimeric protein product (comprising the protein, fragment, analog, or derivative joined via a peptide bond to a heterologous protein sequence). For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification, increase half-life in vivo, and enhance the delivery of an antigen across an epithelial barrier to the immune system. An increase in the half-life in vivo and facilitated purification has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT publications WO 96/22024 and WO 99/04813).

Nucleic acids encoding an API, a fragment of an API; an API-related polypeptide, or a fragment of an API-related polypeptide can be fused to an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897).

An API fusion protein can be made by ligating the appropriate nucleic acid sequences encoding the desired amino acid sequences to each other by methods known in the art, in the proper coding frame, and expressing the chimeric product by methods commonly known in the art. Alternatively, an API fusion protein may be made by protein synthetic techniques, e.g., by use of a peptide synthesizer.

Both cDNA and genomic sequences can be cloned and expressed.

Domain Structure of APIs

Domains of some of the APIs provided by the present invention are known in the art and have been described in the scientific literature. Moreover, domains of an API can be identified using techniques known to those of skill in the art. For. example, one or more domains of an API can be identified by using one or more of the following programs: ProDom, TMpred, and SAPS. ProDom compares the amino acid sequence of a polypeptide to a database of compiled domains (see, e.g., http://www.toulouse.inra.fr/prodom.html; Corpet F., Gouzy J. & Kahn D., 1999, Nucleic Acids Res., 27:263-267). TMpred predicts membrane-spanning regions of a polypeptide and their orientation. This program uses an algorithm that is based on the statistical analysis of TMbase, a database of naturally occuring transmembrane proteins (see, e.g., http://www.ch.embnet.org/software/TMPRED-form.html; Hoffmann & Stoffel. (1993) “TMbase—A database of membrane spanning proteins segments.” Biol. Chem. Hoppe-Seyler 347,166). The SAPS program analyzes polypeptides for statistically significant features like charge-clusters, repeats, hydrophobic regions, compositional domains (see, e.g., Brendel et al., 1992,, Proc. Natl. Acad. Sci. USA 89: 2002-2006). Thus, based on the present description, those skilled in the art can identify domains of an API having enzymatic or binding activity, and further can identify nucleotide sequences encoding such domains; These nucleotide sequences can then be used for recombinant expression of an API fragment that retains the enzymatic or binding activity of the API.

Based on the present description, those skilled in the art can identify domains of an API having enzymatic or binding activity, and further can identify nucleotide sequences encoding such domains. These nucleotide sequences can then be used for recombinant expression of API fragments that retain the enzymatic or binding activity of the API.

In one embodiment, an API has an amino acid sequence sufficiently similar to an identified domain of a known polypeptide. As used herein, the term “functionally similar” refers to a first amino acid or nucleotide sequence which contains a sufficient number of identical or equivalent (e.g., with a similar side chain) amino acid residues or nucleotides to a second amino acid or nucleotide sequence such that the first and second amino acid or nucleotide sequences have or encode a common structural domain or common functional activity or both.

An API domain can be assessed for its function using techniques well known to those of skill in the art. For example, a domain can be assessed for its kinase activity or for its ability to bind to DNA using techniques known to the skilled artisan. Kinase activity can be assessed, for example, by measuring the ability of a polypeptide to phosphorylate a substrate. DNA binding activity can be assessed, for example, by measuring the ability of a polypeptide to bind to a DNA binding element in a electromobility shift assay. In a preferred embodiment, the function of a domain of an API is determined using an assay described in one or more of the references identified in Table IX, infra.

Production of Antibodies to APIs

According to the invention an API, API analog, API-related protein or a fragment or derivative of any of the foregoing may be used as an immunogen to generate antibodies which immunospecifically bind such an immunogen. Such immunogens can be isolated by any convenient means, including the methods described above. Antibodies of the invention include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

In one embodiment, antibodies that recognize gene products of genes encoding APIs may be prepared. For example, antibodies that recognize these APIs and/or their isoforms include antibodies recognizing API-1, API-3, API-4, API-6, API-7, API-10, API-15, API-16, API-22, API-28, API-30, API-33, API-34, API-37, API-38, API-39, API-40, API-42, API-43, API-44, API-45, API-46, AP147, API-50, API-52, API-53, API-55, API-58, API-60, API-62, API-64, API-66, API-67, API-69, API-72, API-74, API-75, API-76, API-77, API-78, API-79, API-80, API-81, API-82, API-84, API-90, API-92, API-93, API-95, API-97, API-98, API-101, API-102, API-103, API-104, API-113, API-118, API-119, API-123, API-124, API-126, API-130, API-131, API-132, API-134, API-136, API-137, API-138, API-140, API-142, API-143, API-145, API-149, API-150, API-161, API-165, API-167, API-168, API-169, API-170, API-171, API-172, API-173, API-174, API-175,.API-176, API-178, API-179, API-181, API-182, API-186, API-188, API-189, API-191, API-194, API-196, API-201, API-215, API-220, API-221, API-223, API-225, or API-233. Certain antibodies are already known and can be purchased from commercial sources as shown in Table VII above. In another embodiment, methods known to those skilled in the art are used to produce antibodies that recognize an API, an API analog, an API-related polypeptide, or a derivative or fragment of any of the foregoing.

In one embodiment of the invention, antibodies to a specific domain of an API are produced. In a specific embodiment, hydrophilic fragments of an API are used as immunogens for antibody production.

In the production of antibodies, screening for the desired antibody can be accomplished by techniques known in the art, e.g. ELISA (enzyme-inked immunosorbent assay). For example, to select antibodies which recognize a specific domain of an API, one may assay generated hybridomas for a product which binds to an API fragment containing such domain. For selection of an antibody that specifically binds a first API homolog but which does not specifically bind to (or binds less avidly to) a second API homolog, one can select on the basis of positive binding to the first API homolog and a lack of binding to (or reduced binding to) the second API homolog. Similarly, for selection of an antibody that specifically binds an API but which does not specifically bind to (or binds less avidly to) a different isoform of the same protein (such as a different glycoform having the same core peptide as the API), one can select on the basis of positive binding to the API and a lack of binding to (or reduced binding to) the different isoform (e.g., a different glycoform). Thus, the present invention provides an antibody (particularly a monoclonal antibody) that binds with greater affinity (particularly at least 2-fold, more particularly at least 5-fold still more particularly at least 10-fold greater affinity) to an API than to a different isoform or isoforms (e.g., glycoforms) of the API.

Polyclonal antibodies which may be used in the methods of the invention are heterogeneous populations of antibody molecules derived from the sera of immunized animals. Unfractionated immune serum can also be used. Various procedures known in the art may be used for the production of polyclonal antibodies to an API, a fragment of an API, an API-related polypeptide, or a fragment of an API-related polypeptide. In a particular embodiment, rabbit polyclonal antibodies to an epitope of an API or an API-related polypeptide can be obtained. For example, for the production of polyclonal or monoclonal antibodies, various host animals can be immunized by injection with the native or a synthetic (e.g., recombinant) version of an API, a fragment of an API, an API-related polypeptide, or a fragment of an API-related polypeptide, including but not limited to rabbits, mice, rats, etc. Isolated APIs suitable for such immunization may be obtained by the use of discovery techniques, such as the preferred technology described herein. If the API is purified by gel electrophoresis, the API can be used for immunization with or without prior extraction from the polyacrylamide gel. Various adjuvants may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum. Additional adjuvants are also well known in the art.

For preparation of monoclonal antibodies (mAbs) directed toward an API, a fragment of an API, an API-related polypeptide, or a fragment of an API-related polypeptide, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Such antibodies may be of any immunoglobulin class including IgG, IgM, IgE, IgA, IgD and any subclass thereof The hybridoma producing the mAbs of the invention may be cultivated in vitro or in vivo. In an additional embodiment of the invention, monoclonal antibodies can be produced in germ-free animals utilizing known technology (PCT/US90/02545, incorporated herein by reference).

The monoclonal antibodies include but are not limited to human monoclonal antibodies and chimeric monoclonal antibodies (e.g., human-mouse chimeras). Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060.

Completely human antibodies (antibodies derived solely from human antigenic material) are particularly desirable for therapeutic treatment of human subjects. Such antibodies can be produced using transgenic mice which are incapable of expressing endogenous inununoglobulin heavy and light chains genes, but which can express human heavy and light chain genes. The transgenic mice are immunized in the normal fashion with selected antigens, e.g., all or a portion of an API of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Biotechnology 12:899-903).

The antibodies of the present invention can also be generated using various phage display methods known in the art. In phage display methods, functional antibody domains are displayed on the surface of phage particles which carry the polynucleotide sequences encoding them. In a particular, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VII protein. Phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of suitable techniques which can be used to produce single-chain Fvs and antibodies against APIs of the present invention include those described in U.S. Pat. Nos. 4,946,778 and 5,258,498; Huston et al., Methods in Enzymology 203:46-88 (1991); Shu et al., PNAS 90:7995-7999 (1993), and Skerra et al., Science 240:1038-1040 (1988).

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al., 1983, Nature 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published 13 May 1993, and in Traunecker et al., 1991, EMBO J. 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an inmnunoglobulin light chain in only one half of the bispecific molecule provides for a facile way of separation. This approach is disclosed in WO 94/04690 published March 3,1994. For further details for generating bispecific antibodies see, for example, Suresh et al., Methods in Enzymology,1986, 121:210.

The invention provides functionally active fragments, derivatives or analogs of the anti-API immunoglobulin molecules. Functionally active means that the fragment, derivative or analog is able to elicit anti-anti-idiotype antibodies (i.e., tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any suitable binding assay known in the art.

The present invention provides antibody fragments such as, but not limited to, F(ab′)2 fragments and Fab fragments. Antibody fragments which recognize specific epitopes may be generated by known techniques. F(ab′)2 fragments consist of the variable region, the light chain constant region and the CH1 domain of the heavy chain and are generated by pepsin digestion of the antibody molecule. Fab fragments are generated by reducing the disulfide bridges of the F(ab′)2 fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al., 1988, Science 242:1038-1041).

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, preferably at least 10, 20 or 5.0 amino acid poirtion of the protein) that is not the immunoglobulin. Preferably the immunoglobulin, or fragment thereof, is covalently linked to the other protein at the N-terninus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are either modified, i.e, by the covalent attachment of any type of molecule as long as such covalent attachment that does not impair inununospecific binding. For example, but not by way of limitation, the derivatives and analogs of the immunoglobulins include those that have been further modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical or unnatural amino acids.

The foregoing antibodies can be used in methods known in the art relating to the localization and activity of the APIs of the invention, e.g., for imaging these proteins, measuring levels thereof in appropriate physiological samples, in diagnostic methods, etc.

Expression of Antibodies

The antibodies of the invention can be produced by any suitable method known in the art for the synthesis of antibodies, in particular, by chemical synthesis or by recombinant expression, and are preferably produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al., 1994, BioTechniques 17:242), which, briefly, involves. the synthesis of overlapping oligonucleotides containing portions of the sequence encoding. antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridizable to the 3′ and 5′ ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating monoclonal antibodies. Alternatively, a clone encoding at least the Fab portion of the antibody may be obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Once a nucleic acid encoding at least the variable domain of the antibody molecule is obtained, it may be introduced into a vector containing the nucleotide sequence encoding the constant region of the antibody molecule (see, e.g., PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis, in vitro site directed mutagenesis (Hutchinson et al., 1978, J. Biol. Chem. 253:6551), PCT based methods, etc.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g., humanized antibodies.

Once a nucleic acid encoding an antibody molecule of the invention has been obtained, the vector for the production of the antibody molecule may be produced by recombinant DNA technology using techniques well known in the art. Thus, methods for preparing the protein of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al. (1990, Molecular Cloning, A Laboratory Manual, 2d Ed., Cold Spring Harbor Laboratory, Cold Spring Harbor, N.Y.) and Ausubel et al. (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; Cockett et al., 1990, Bio/Technology 8:2).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al., 1983, EMBO J. 2:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa califormica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g., an adenovirus expression system) may be utilized.

As discussed above, a host cell strain may be chosen based on the present description which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cells lines that stably express an antibody of interest can be produced by transfecting the cells with an expression vector comprising the nucleotide sequence of the antibody and the nucleotide sequence of a selectable. (e.g., neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, 1986, Nature 322:52; Kohler, 1980, Proc. Natl. Acad. Sci. USA 77:2197). The coding sequences for the heavy and light chains may comprise cDNA or genomic DNA.

Once the antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni2+ nitriloacetic acid-agarose columns and histidine-tagged proteins are selectively eluted with imidazole-containing buffers.

Conjugated Antibodies

In a preferred embodiment, anti-API antibodies or fragments thereof are conjugated to a diagnostic or a therapeutic molecule. The antibodies can be used, for example, for diagnosis or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 125I, 131I, 111In and 99Tc.

An anti-API antibodies or fragments thereof can be conjugated to a therapeutic agent or pharmaceutical product to modify a given biological response. The therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, e.g., angiostatin or endostatin; or, a biological response modifier such as a lymphokine, interleukin-1 (IL-1), interleukin-2 (IL-2), interleukin-⁶ (IL-6), granulocyte macrophage colony stimulating factor (GM-CSF), granulocyte colony stimulating factor (G-CSF), nerve growth factor (NGF) or other growth factor.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). These references are incorporated herein in their entirety.

Alternatively, an antibody can be conjugated to a second antibody to form an antibody heteroconjugate as described by Segal in U.S. Pat. No. 4,676,980.

An antibody with or without a therapeutic moiety conjugated to it can be used as a therapeutic that is administered alone or in combination with cytotoxic factor(s) and/or cytokine(s).

Diagnosis of Alzheimer's Disease

In accordance with the present invention, suitable test samples, e.g., of cerebrospinal fluid (CSF), serum, plasma or urine obtained from a subject suspected of having or known to have Alzheimer's disease can be used for diagnosis. In one embodiment, a decreased abundance of one or more AFs or APIs (or any combination of them) in a test sample relative to a control sample (from a subject or subjects free from Alzheimer's disease) or a previously determined reference range indicates the presence of Alzheimer's disease; AFs and APIs suitable for this purpose are identified in Tables I and IV, respectively, as described in detail above. In another embodiment of the invention, an increased abundance of one or more AFs or APIs (or any combination of them) in a test sample compared to a control sample or a previously determined reference range indicates the presence of Alzheimer's disease; AFs and APIs suitable for this purpose are identified in Tables II and V, respectively, as described in detail above. In another embodiment, the relative abundance of one or more AFs or APIs (or any combination of them) in a test sample compared to a control sample or a previously determined reference range indicates a subtype of Alzheimer's disease (e.g., familial or sporadic Alzheimer's disease). In yet another embodiment, the relative abundance of one or more AFs or APIs (or any combination of them) in a test sample relative to a control sample or a previously determined reference range indicates the degree or severity of Alzheimer's disease. In any of the aforesaid methods, detection of one or more APIs described herein may optionally be combined with detection of one or more additional biomarkers for Alzheimer's disease including, but not limited to apoplipoprotein E (ApoE), amyloid β-peptides (AP), tau and neural thread protein (NTP). Any suitable method in the art can be employed to measure the level of AFs and APIs, including but not limited to the Preferred Technology described herein, kinase assays, immunoassays to detect and/or visualize the API (e.g., Western blot, immunoprecipitation followed by sodium dodecyl sulfate polyacrylamide gel electrophoresis, immunocytochemistry, etc.). In cases where an API has a known function, an assay for that function may be used to measure API expression. In a further embodiment, a decreased abundance of mRNA encoding one or more APIs identified in Table IV (or any combination of them) in a test sample relative to a control sample or a previously determined reference range indicates the presence of Alzheimer's disease. In yet a further embodiment, an increased abundance of mRNA encoding one or more APIs identified in Table V (or any combination of them) in a test sample relative to a control sample or previously determined reference range indicates the presence of Alzheimer's disease. Any suitable hybridization assay can be used to detect API expression by detecting and/or visualizing mRNA encoding the API (e.g., Northern assays, dot blots, in situ hybridization, etc.).

In another embodiment of the invention, labeled antibodies, derivatives and analogs thereof, which specifically bind to an API can be used for diagnostic purposes, e.g., to detect, diagnose,,or monitor Alzheimer's disease. Preferably, Alzheimer's disease is detected in an animal, more preferably in a mammal and most preferably in a human.

Screening Assays

The invention provides methods for identifying agents (e.g., chemical compounds, proteins, or peptides) that bind to an API or have a stimulatory or inhibitory effect on the expression or activity of an API. The invention also provides methods of identifying agents, candidate compounds or test compounds that bind to an API-related polypeptide or an API fusion protein or have a stimulatory or inhibitory effect on the expression or activity of an API-related polypeptide or an API fusion protein. Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous suitable approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No.5,807,683, each of which is incorporated herein in its entirety by reference).

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233, each of which is incorporated herein in its entirety by reference.

Libraries of compounds may be presented, e.g., presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310), each of which is incorporated herein in its entirety by reference.

In one embodiment, agents that interact with (i.e., bind to) an API, an API fragment (e.g. a functionally active fragment), an API-related polypeptide, a fragment of an API-related polypeptide, or an API fusion protein are identified in a cell-based assay system. In accordance with this embodiment, cells expressing an API, a fragment of an API, an API-related polypeptide, a fragment of an API-related polypeptide, or an API fusion protein are contacted with a candidate compound or a control compound and the ability of the candidate compound to interact with the API is determined. If desired, this assay may be used to screen a plurality (e.g. a library) of candidate compounds. The cell, for example, can be of prokaryotic origin (e.g., E. coli) or eukaryotic origin (e.g., yeast or mammalian). Further, the cells can express the API, fragment of the API, API-related polypeptide, a fragment of the API-related polypeptide, or an API fusion protein endogenously or be genetically engineered to express the API, fragment of the API, API-related polypeptide, a fragment of the API-related polypeptide, or an API fusion protein. In some embodiments, the API, fragment of the API, API-related polypeptide, a fragment of the API-related polypeptide, or an API fusion protein or the candidate compound is labeled, for example with a radioactive label (such as 32P, 35S or 125I) or a fluorescent label (such as fluorescein isothiocyanate, rhodamine, phycoerythrin, phycocyanin, allophycocyanin, o-phthaldehyde or fluorescamine) to enable detection of an interaction between an API and a candidate compound. The ability of the candidate compound to interact directly or indirectly with an API, a fragment of an API, an API-related polypeptide, a fragment of an API-related polypeptide, or an API fusion protein can be determined by methods known to those of skill in the art. For example, the interaction between a candidate compound and an API, a fragment of an API, an API-related polypeptide, a fragment of an API-related polypeptide, or an API fusion protein can be determined by flow cytometry, a scintillation assay, immunoprecipitation or western blot analysis.

In another embodiment, agents that interact with (i.e., bind to) an API, an API fragment (e.g., a functionally active fragment) an API-related polypeptide, a fragment of an API-related polypeptide, or an API fusion protein are identified in a cell-free assay system. In accordance with this embodiment, a native or recombinant API or fragment thereof, or a native or recombinant API-related polypeptide or fragment thereof, or an API-fusion protein or fragment thereof, is contacted with a candidate compound or a control compound and the ability of the candidate compound to interact with the API or API-related polypeptide, or API fusion protein is determined. If desired, this assay may be used to screen a plurality (e.g. a library) of candidate compounds. Preferably, the API, API fragment, API-related polypeptide, a fragment of an API-related polypeptide, or an API-fusion protein is first immobilized, by, for example, contacting the API, API fragment, API-related polypeptide, a fragment of an API-related polypeptide, or an API fusion protein with an inmobilized antibody which specifically recognizes and binds it, or by contacting a purified preparation of the API, API fragment, API-related polypeptide, fragment of an API-related polypeptide, or an API fusion protein with a surface designed to bind proteins. The API, API fragment, API-related polypeptide, a fragment of an API-related polypeptide, or an API fusion protein may be partially or completely purified (e.g., partially or completely free of other polypeptides) or part of a cell lysate. Further, the API, API fragment, API-related polypeptide, a fragment of an API-related polypeptide may be a fusion protein comprising the API or a biologically active portion thereof, or API-related polypeptide and a domain such as glutathionine-S-transferase. Alternatively, the API, API fragment, API-related polyp eptide, fragment of an API-related polypeptide or API fusion protein can be biotinylated using techniques well known to those of skill in the art (e.g., biotinylation kit, Pierce Chemicals; Rockford, Ill.). The ability of the candidate compound to interact with an API, API fragment, API-related polypeptide, a fragment of an API-related polypeptide, or an API fusion protein can be can be determined by methods known to those of skill in the art.

In another embodiment, a cell-based assay system is used to identify agents that bind to or modulate the activity of a protein, such as an enzyme, or a biologically active portion thereof, which is, responsible for the production or degradation of an API or is responsible for the post- translational modification of an API. In a primary screen, a plurality (e.g., a library) of compounds are contacted with cells that naturally or recombinantly express: (i) an API, an isoform of an API, an API homolog an API-related polypeptide, an API fusion protein, or a biologically active fragment of any of the foregoing; and (ii) a protein that is responsible for processing of the API, API isoform, API homolog, API-related polypeptide, API fusion protein, or fragment in order to identify compounds that modulate the production, degradation, or post-translational modification of the API, API isoform, API homolog, API-related polypeptide, API fusion protein or fragment. If desired, compounds identified in the primary screen can then be assayed in a secondary screen against cells naturally or recombinantly expressing the specific API of interest. The ability of the candidate compound to modulate the production, degradation or post-translational modification of an API, isoform, homolog, API-related polypeptide, or API fusion protein-can be determined by methods known to those of skill in the art, including without limitation, flow cytometry, a scintillation assay, immunoprecipitation and western blot analysis.

In another embodiment, agents that competitively interact with (i.e., bind to) an API, API fragment, API-related polypeptide, a fragment of an API-related polypeptide, or an API fusion protein are identified in a competitive binding assay. In accordance with this embodiment, cells expressing an API, API fragment, API-related polypeptide, a fragment of an API-related polypeptide, or an API fusion protein are contacted with a candidate compound and a compound known to interact with the API, API fragment, API-related polypeptide, a fragment of an API-related polypeptide or an API fusion protein; the ability of the candidate compound to competitively interact with the API, API fragment, API-related polypeptide, fragment of an API-related polypeptide, or an API fusion protein is then determined. Alternatively, agents that competitively interact with (i.e., bind to) an API, API fragment, API-related polypeptide or fragment of an API-related polypeptide are identified in a cell-free assay system by contacting an API, API fragment, API-related polypeptide, fragment of an API-related polypeptide, or an API fusion protein with a candidate compound and a compound known to interact with the API, API-related polypeptide or API fusion protein. As stated above, the ability of the candidate compound to interact with an API, API fragment, API-related polypeptide, a fragment of an API-related polypeptide, or an API fusion protein can be determined by methods known to those of skill in the art. These assays, whether cell-based or cell-free, can be used to screen a plurality (e.g., a library) of candidate compounds.

In another embodiment, agents that modulate (i.e., upregulate or downregulate) the expression of an API, or an API-related polypeptide are identified by contacting cells (e.g., cells of prokaryotic origin or eukaryotic origin) expressing the API, or API-related polypeptide with a candidate compound or a control compound (e.g., phosphate buffered saline (PBS)) and determining the expression of the API, API-related polypeptide, or API fusion protein, mRNA encoding the API, or mRNA encoding the API-related polypeptide. The level of expression of a selected API, API-related polypeptide, mRNA encoding the API, or mRNA encoding the API-related polypeptide in the presence of the candidate compound is compared to the level of expression of the API, API-related polypeptide, mRNA encoding the API, or mRNA encoding the API-related polypeptide in the absence of the candidate compound (e.g., in the presence of a control compound). The candidate compound can then be identified as a modulator of the expression of the API, or an API-related polypeptide based on this comparison. For example, when expression of the API or mRNA is significantly greater in the presence of the candidate compound than in its absence, the candidate compound is identified as a stimulator of expression of the API or mRNA. Alternatively, when expression of the API or mRNA is significantly less in the presence of the candidate compound than in its absence, the candidate compound is identified as an inhibitor of the expression of the API or mRNA. The level of expression of an API or the mRNA that encodes it can be determined by methods known to those of skill in the art based on the present description. For example, mRNA expression-can be assessed by Northern blot analysis or RT-PCR, and protein levels can be assessed by western blot analysis.

In another embodiment, agents that modulate the activity of an API, or an API-related polypeptide are identified by contacting a preparation containing the API or API-related polypeptide, or cells (e.g., prokaryotic or eukaryotic cells) expressing the API or API-related polypeptide with a test compound or a control compound and determining the ability of the test compound to modulate (e.g., stimulate or inhibit) the activity of the API or API-related polypeptide. The activity of an API or an API-related polypeptide can be assessed by detecting induction of a cellular signal transduction pathway of the API or API-related polypeptide (e.g. , intracellular Ca2+, diacylglycerol, IP3, etc.), detecting catalytic or enzymatic activity of the target on a suitable substrate, detecting the induction of a reporter gene ( e.g., a regulatory element that is responsive to an API or an API-related polypeptide and is operably linked to a nucleic acid encoding a detectable marker, e.g., luciferase), or detecting a cellular response, for example, cellular differentiation, or cell proliferation as the case may be, based on the present description, techniques known to those of skill in the art can be used for measuring these activities. (see, e.g., U.S. Pat. No. 5,401,639, which is incorporated in its entirety herein by reference). The candidate agent can then be identified as a modulator of the activity of an API or API-related polypeptide by comparing the effects of the candidate compound to the control compound. Suitable control compounds include phosphate buffered saline (PBS) and normal saline (NS).

In another embodiment, agents that modulate (i.e., upregulate or downregulate) the expression, activity or both the expression and activity of an API or API-related polypeptide are identified in an animal model. Examples of suitable animals include, but are not limited to, mice, rats, rabbits, monkeys, guinea pigs, dogs and cats. Preferably, the animal used represent a model of Alzheimer's disease (e.g., animals that express human familial Alzheimer's disease (FAD) β-amyloid precursor (APP), animals that overexpress human wild-type APP, animals that overexpress β-amyloid 1-42 (βA), animals that express FAD presenillin-I (PS-1). See, e.g., Higgins, LS, 1999, Molecular Medicine Today 5:274-276. In accordance with this embodiment, the test compound or a control compound is administered (e.g., orally, rectally or parenterally such as intraperitoneally or intravenously) to a suitable animal and the effect on the expression, activity or both expression and activity of the API or API-related polypeptide is determined. Changes in the expression of an API or API-related polypeptide can be assessed by any suitable method described above, based on the present description.

In yet another embodiment, an API or API-related polypeptide is used as a “bait protein” in a two-hybrid assay or three hybrid assay to identify other proteins that bind to or interact with an API or API-related polypeptide (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300). As those skilled in the art will appreciate, such binding proteins are also likely to be involved in the propagation of signals by the APIs of the invention as, for example, upstream or downstream elements of a signaling pathway involving the APIs of the invention.

As those skilled in the art will appreciate, Table X enumerates scientific publications describing suitable assays for detecting or quantifying enzymatic or binding activity of an API, an API analog, an API-related polypeptide, or a fragment of any of the foregoing. Each such reference is hereby incorporated in its entirety. In a preferred embodiment, an assay referenced in Table X is used in the screens and assays described herein, for example, to screen for or to identify an agent that modulates the activity of (or that modulates both the expression and activity of) an API, API analog, or API-related polypeptide, a fragment of any of the foregoing or an API fusion protein.

TABLE X

| API | References |
|---|---|
| API-39, | Structural Biology 7, 312-321, 2000 |
| API-44, | J. Am. Chem. Soc. 122, 2178-2192, 2000 |
| API-178, | |
| API-188 | |
| API-76 | Clin Chem 1993 Feb 39: 2 309-12 |
| API-78 | J Immunol Methods 1987 Aug 24 102: 1 7-14 |
| API-79 | |
| API-80 | |
| API-82 | |
| API-140 | |
| API-38 | J Clin Lab Immunol 1986 Dec 21: 4 201-7 |
| API-74 | |
| API-105 | |
| API-124 | |
| API-130 | |
| API-138 | |
| API-169 | |
| API-172 | |
| API-123 | Neuroendocrinology 1992 Mar 55: 3 308-16 |
| API-126 | |
| API-186 | J Chromatogr 1991 Jul 5 567: 2 369-80; |
| | Clin Chem 1989 Apr 35: 4 582-6 |
| API-52 | J Chromatogr 1987 Dec 18 411: 498-501 |
| | Eisei Shikenjo Hokoku 1972 90: 89-92 |
| | Analyst 1990 Aug 115: 8 1143-4 |
| API-182 | Biochem J 1997 Mar 1 322 (Pt 2): 455-60; |
| | Biochem Soc Trans 1997 Nov 25: 4 S591; |
| | Biochim Biophys Acta 1986 Oct 10 888: 3 325-31 |
| | http://www.promega.com |

This invention further provides novel agents identified by the above-described screening assays and uses thereof for treatments as described herein.

Therapeutic uses of APIs

The invention provides for treatment or prevention of various diseases and disorders by administration of a therapeutic agent. Such agents include but are not limited to: APIs, API analogs, API-related polypeptides and derivatives (including fragments) thereof; antibodies to the foregoing; nucleic acids encoding APIs, API analogs, API-related polypeptides and fragments thereof; antisense nucleic acids to a gene encoding an API or API-related polypeptide; and modulator (e.g., agonists and antagonists) of a gene encoding an API or API-related polypeptide. An important feature of the present invention is the identification of genes encoding APIs involved in Alzheimer's disease. Alzheimer's disease can be treated (e.g. to ameliorate symptoms or to retard onset or progression) or prevented by administration of a therapeutic compound that promotes function or expression of one or more APIs that are decreased in the CSF of Alzheimer's disease subjects having Alzheimer's disease, or by administration of a therapeutic compound that reduces function or expression of one or more APIs that are increased in the CSF of subjects having Alzheimer's disease.

In one embodiment, one or more antibodies each specifically binding to an API are administered alone or in combination with one or more additional therapeutic compounds or treatments. Examples of such therapeutic compounds or treatments include, but are not limited to, tacrine, donepezil, α-tocopherol, selegeline, NSAIDs, estrogen replacement therapy, physostigrnine, rivastigmine, hepastigmine, metrifonate, ENA-713, ginkgo biloba extract, physostigmine, arridin, talsaclidine, zifrosilone, eptastigmine, methanesulfonyl chloride, nefiracetam, ALCAR, talsachidine, xanomeline, galanthamine, and propentofylline.

Preferably, a biological product such as an antibody is allogeneic to the subject to which it is administered. In a preferred embodiment, a human API or a human API-related polypeptide, a nucleotide sequence encoding a human API or a human API-related polypeptide, or an antibody to a human API or a human API-related polypeptide, is administered to a human subject for therapy (e.g. to ameliorate symptoms or to retard onset or progression) or prophylaxis.

Treatment and Prevention of Alzheimer's Disease

Alzheimer's disease can be treated or prevented by administration to a subject suspected of having or known to have Alzheimer's disease or to be at risk of developing Alzheimer's disease following administration of an agent that modulates (i.e., increases or decreases) the level or activity (i.e., function) of one or more APIs or the level of one or more AFs that are differentially present in the CSF of subjects having Alzheimer's disease compared with CSF of subjects free from Alzheimer's disease. In one embodiment, Alzheimer's disease is treated by administering to a subject suspected of having or known to have Alzheimer's disease or to be at risk of developing Alzheimer's disease following administration of an agent that upregulates (i.e., increases) the level or activity (i.e., function) of one or more APIs or the level of one or more AFs that are decreased in the CSF of subjects having Alzheimer's disease. In another embodiment, an agent is administered that upregulates the level or activity (i.e., function) of one or more APIs or the level of one or more AFs that are increased in the CSF of subjects having Alzheimer's disease. Examples of such a compound include but are not limited to: APIs, API fragments and API-related polypeptides; nucleic acids encoding an API, an API fragment and an API-related polypeptide (e.g., for use in gene therapy); and, for those APIs or API-related polypeptides with enzymatic activity, compounds or molecules known to modulate that enzymatic activity. Other compounds that can be used, e.g., API agonists, can be identified using in vitro assays, as defined or described above or earlier.

Alzheimer's disease is also treated or prevented by administration to a subject suspected of having or known to have Alzheimer's disease or to be at risk of developing Alzheimer's disease of a compound that downregulates the level or activity of one or more APIs or the level of one or more AFs that are increased in the CSF of subjects having Alzheimer's disease. In another embodiment, a compound is administered that downregulates the level or activity of one or more APIs or the level of one or more AFs that are decreased in the CSF of subjects having Alzheimer's disease. Examples of such a compound include, but are not limited to, API antisense oligonucleotides, ribozymes, antibodies directed against APIs, and compounds that inhibit the enzymatic activity of an API. Other useful compounds e.g. API antagonists and small molecule API antagonists, can be identified using in vitro assays.

In a preferred embodiment, therapy or prophylaxis is tailored to the needs of an individual subject. Thus, in specific embodiments, compounds that promote the level or function of one or more APIs, or the level of one or more AFs, are therapeutically or prophylactically administered to a subject suspected of having or known to have Alzheimer's disease, in whom the levels or functions of said one or more APIs, or levels of said one or more AFs, are absent or are decreased relative to a control or normal reference range. In further embodiments, compounds that promote the level or function of one or more APIs, or the level of one or more AFs, are therapeutically or prophylactically administered to a subject suspected of having or known to have Alzheimer's disease in whom the levels or functions of said one or more APIs, or levels of said one or more AFs, are increased relative to a control or to a reference range. In further embodiments, compounds that decrease the level or function of one or more APIs, or the level of one or more AFs, are therapeutically or prophylactically administered to a subject suspected of having or known to have Alzheimer's disease in whom the levels or functions of said one or more APIs, or levels of said one or more AFs, are increased relative to a control or to a reference range. In further embodiments, compounds that decrease the level or function of one or more APIs, or the level of one or more AFs, are therapeutically or prophylactically administered to a subject suspected of having or known to have Alzheimer's disease in whom the levels or functions of said one or more APIs, or levels of said one or more AFs, are decreased relative to a control or to a reference range. The change in API function or level, or AF level, due to the administration of such compounds can be readily detected, e.g., by obtaining a sample (e.g., a sample of CSF, blood or urine or a tissue sample such as biopsy tissue) and assaying in vitro the levels of said AFs or the levels or activities of said APIs, or the levels of mRNAs encoding said APIs. or any combination of the foregoing. Such assays can be performed before and after the administration of the compound as described herein.

The compounds of the invention include but are not limited to any compound, e.g., a small organic molecule, protein, peptide, antibody, nucleic acid, etc. that restores the Alzheimer's disease API or AF profile towards normal with the proviso that such compound is not an acetylcholinesterase (AChE) inhibitor (e.g., tacrine, donepezil, rivastigmine, hepastigmine, Metrigonate, physostigmine, Amridin, Talsaclidine, KA-672, Huperzine, P-11012, P-11149, Zifrosilone, Eptastigmine, Methanesulfonyl chloride, and S-9977), an acetylcholine receptor agonist (e.g.. Nefiracetam, LU-25109, and NS2330), a muscarinic receptor agonist (e.g., SB-20206, Talsachidine, AF-1025B, and SR-46559A), a nicotonic cholinergic receptor agonist (e.g., ABT-418), an acetylcholine modulator.(e.g., FKS-508 and Galantamine) or propentofylline.

Gene Therapy

In another embodiment, nucleic acids comprising a sequence encoding an API, an API fragment, API-related polypeptide or fragment of an API-related polypeptide, are administered to promote API function by way of gene therapy. Gene therapy refers to the administration of an expressed or expressible nucleic acid to a subject. In this embodiment, the nucleic acid produces its encoded polypeptide and the polypeptide mediates a therapeutic effect by promoting API function.

Any suitable methods for gene therapy available in the art can be used according to the present invention.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used in the present invention are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In a particular aspect, the compound comprises a nucleic acid encoding an API or fragment or chimeric protein thereof, said nucleic acid being part of an expression vector that expresses an API or fragment or chimeric protein thereof in a suitable host. In particular, such a nucleic acid has a promoter operably linked to the API coding region, said promoter being inducible or constitutive (and, optionally, tissue-specific). In another particular embodiment, a nucleic acid molecule is used in which the API coding sequences and any other desired sequences are flanked by regions that promote homologous recombination at a desired site in the genome, thus providing for intrachromosomal expression of the API nucleic acid (Koller and Smithies, 1989, Proc. Natl. Acad. Sci. USA 86:8932-8935; Zijlstra et al., 1989, Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomaI degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated December 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a further embodiment, a viral vector that contains a nucleic acid encoding an API is used. For example, a retroviral vector can be used (see Miller et al., 1993, Meth. Enzymol. 217:581-599). These retroviral vectors have been modified to delete retroviral sequences that are not necessary for packaging of the viral genome and integration into host cell DNA. The nucleic acid encoding the API to be used in gene therapy is cloned into the vector, which facilitates delivery of the gene into a subject. More detail about retroviral vectors can be found in Boesen et al., 1994, Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang, et al., 1995, Gene Therapy 2:775-783.

Adeno-associated virus (AAV) has also been proposed for use in gene therapy (Walsh et al., 1993, Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genies into cells (see, e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject. Recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver.

In a preferred embodiment, the cell used for gene therapy is autologous to the subject that is treated.

In an embodiment in which recombinant cells are used in gene therapy, a nucleic acid encoding an API is introduced into the cells such that it is expressible by the cells or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. PCT Publication WO 94/08598, dated Apr. 28, 1994; Stemple and Anderson, 1992, Cell 71:973-985; Rheinwald, 1980, Meth. Cell Bio. 21A:229; and Pittelkow and Scott, 1986, Mayo Clinic Proc. 61:771).

In another embodiment, the nucleic acid to be introduced for purposes of gene therapy may comprises an inducible promoter operably linked to the coding region, such that expression of the nucleic acid is controllable by controlling the presence or absence of the appropriate inducer of transcription.

Direct injection of a DNA coding for an API may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection and the elicitation of an immune response in the subject to the protein encoded by the injected DNA. In a preferred embodiment, naked DNA comprising (a) DNA encoding an API and (b) a promoter are injected into a subject to elicit an immune response to the API.

Inhibition of APIs to Treat Alzheimer's Disease

In one embodiment of the invention, Alzheimer's disease is treated or prevented by administration of a compound that antagonizes (inhibits) the level(s) and/or function(s) of one or more APIs which are elevated in the CSF of subjects having Alzheimer's disease as compared with CSF of subjects free from Alzheimer's disease. Compounds useful for this purpose include but are not limited to anti-API antibodies (and fragments and derivatives containing the binding region thereof), API antisense or ribozyme nucleic acids, and nucleic acids encoding dysfunctional APIs that are used to “knockout” endogenous API function by homologous recombination (see, e.g., Capecchi, 1989, Science 244:1288-1292). Other compounds that inhibit API function can be identified by use of known in vitro assays, e.g., assays for the ability of a test compound to inhibit binding of an API to another protein or a binding partner, or to inhibit a known API function. Preferably such inhibition is assayed in vitro or in cell culture, but genetic assays may also be employed. The Preferred Technology can also be used to detect levels of the API before and after the administration of the compound. Preferably, suitable in vitro or in vivo assays are utilized to determine the effect of a specific compound and whether its administration is indicated for treatment of the affected tissue, as described in more detail below.

In a particular embodiment, a compound that inhibits an API function is administered therapeutically or prophylactically to a subject in whom an increased CSF level or functional activity of the API (e.g., greater than the normal level or desired level) is detected as compared with CSF of subjects free from Alzhleimer's disease or a predetermined reference range. Methods standard in the art can be employed to measure the increase in an API level or function, as outlined above. Preferred API inhibitor compositions include small molecules, i.e., molecules of 1000 daltons or less. Such small molecules can be identified by the screening methods described herein.

Antisense Regulation of APIs

In a further embodiment, API expression is inhibited by use of API antisense nucleic acids. The present invention provides the therapeutic or prophylactic use of nucleic acids comprising at least six nucleotides that are antisense to a gene or cDNA encoding an API or a portion thereof. As used herein, an API “antisense” nucleic acid refers to a nucleic acid capable of hybridizing by virtue of some sequence complementarity to a portion of an RNA (preferably mRNA) encoding an API. The antisense nucleic acid may be complementary to a coding and/or noncoding region of an mRNA encoding an API. Such antisense nucleic acids have utility as compounds that inhibit API expression, and can be used in the treatment or prevention of Alzheimer's disease.

The antisense nucleic acids of the invention are double-stranded or single-stranded oligonucleotides, RNA or DNA or a modification or derivative thereof, and can be directly administered to a cell or produced intracellularly by transcription of exogenous, introduced sequences.

The invention further provides pharmaceutical compositions comprising a therapeutically effective amount of an API antisense nucleic acid, and a pharmaceutically-acceptable carrier, vehicle or diluent.

In another embodiment, the invention provides methods for inhibiting the expression of an API nucleic acid sequence in a prokaryotic or eukaryotic cell comprising providing the cell with an effective amount of a composition comprising an API antisense nucleic acid of the invention.

API antisense nucleic acids and their uses are described in detail below.

API Antisense Nucleic Acids

The API antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single-stranded or double-stranded. The oligonucleotide can be modified at the base moiety, sugar moiety, or phosphate backbone. The oligonucleotide may include other appended groups such as peptides; agents that facilitate transport across the cell membrane (see, e.g., Letsinger et al., 1989, Proc. Natl. Acad. Sci. USA 86:6553-6556; Lemaitre et al., 1987, Proc. Natl. Acad. Sci. 84:648-652; PCT Publication No. WO 88/09810, published Dec. 15, 1988) or blood-brain barrier (see, e.g., PCT Publication No. WO 89/10134, published Apr. 25, 1988); hybridization-triggered cleavage agents (see, e.g., Krol et al., 1988, BioTechniques 6:958-976) or intercalating agents (see, e.g., Zon, 1988, Pharm. Res. 5:539-549).

In a particular aspect of the invention, an API antisense oligonucleotide is provided, preferably of single-stranded DNA. The oligonucleotide may be modified at any position on its structure with substituents generally known in the art.

The API antisense oligonucleotide may comprise any suitable of the following modified base moieties, e.g. 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5 -methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, and other base analogs.

In another embodiment, the oligonucleotide comprises at least one modified sugar moiety, e.g., one of the following sugar moieties: arabinose, 2-fluoroarabinose, xylulose, and hexose.

In yet another embodiment, the oligonucleotide comprises at least one of the following modified phosphate backbones: a phosphorothioate, a phosphorodithioate, a phosphoramidothioate, a phosphoramidate, a phosphordiamidate, a methylphosphonate, an alkyl phosphotriester, a formacetal, or an analog of formacetal.

In yet another embodiment, the oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15:6625-6641).

The oligonucleotide may be conjugated to another molecule, e.g., a peptide, hybridization triggered cross-linking agent, transport agent, or hybridization-triggered cleavage agent.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), and methylphosphonate oligonucleotides can be prepared by use of controlled pore glass polymer supports (Sarin et al., 1988, Proc. Natl. Acad. Sci. USA 85:7448-7451).

In another embodiment, the API antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the API antisense nucleic acid. Such a vector can remain episomal or become chromosomally integrated, as long as it can be transcribed to produce the desired antisense RNA. Such vectors can be constructed by recombinant DNA technology standard in the art. Vectors can be plasmid, viral, or others known in the art, used for replication and expression in mammalian cells. Expression of the sequence encoding the API antisense RNA can be by any promoter known in the art to act in mammalian, preferably human, cells. Such promoters can be inducible or constitutive. Examples of such promoters are outlined above.

The antisense nucleic acids of the invention comprise a sequence complementary to at least a portion of an RNA transcript of a gene encoding an API, preferably a human gene encoding an API. However, absolute complementarity, although preferred, is not required. A sequence “complementary to at least a portion of an RNA,” as referred to herein, means a sequence having sufficient complementarity to be able to hybridize under stringent conditions (e.g. highly stringent conditions comprising hybridization in 7% sodium dodecyl sulfate (SDS), 1 mM EDTA at 65 C and washing in 0.1×SSC/0.1% SDS at 68 C, or moderately stringent conditions comprising washing in 0.2×SSC/0.1%; SDS at 42 C ) with the RNA, forming a stable duplex; in the case of double-stranded API antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA encoding an API it may contain and still form a stable duplex (or triplex, as the case may be). One skilled in the art can ascertain a tolerable degree of mismatch by use of standard procedures to determine the melting point of the hybridized complex.

Therapeutic use of API Antisense Nucleic Acids

The API antisense nucleic acids can be used to treat or prevent Alzheimer's disease when the target API is overexpressed in the CSF of subjects suspected of having or suffering from Alzheimer's disease. In a preferred embodiment, a single-stranded DNA antisense API oligonucleotide is used.

Cell types which express or overexpress RNA encoding an API can be identified by various methods known in the art. Such cell types include but are not limited to leukocytes (e.g., neutrophils, macrophages, monocytes) and resident cells (e.g., astrocytes, glial cells, neuronal cells, and ependymal cells). Such methods include, but are not limited to, hybridization with an API-specific nucleic acid (e.g. by Northern hybridization, dot blot hybridization, in situ hybridization), observing the ability of RNA from the cell type to be translated in vitro into an API, immunoassay, etc. In a preferred aspect, primary tissue from a subject can be assayed for API expression prior to treatment, e.g., by immunocytochemistry or in-situ hybridization.

Pharmaceutical compositions of the invention, comprising an effective amount of an API antisense nucleic acid in a pharmaceutically acceptable carrier, vehicle or diluent can be administered to a subject having Alzheimer's disease.

The amount of API antisense nucleic acid which will be effective in the treatment of Alzheimer's disease can be determined by standard clinical techniques.

In a specific embodiment, pharmaceutical compositions comprising one or more API antisense nucleic acids are administered via liposomes, microparticles, or microcapsules. In various embodiments of the invention, such compositions may be used to achieve sustained release of the API antisense nucleic acids.

Inhibitory Ribozyme and Triple Helix Approaches

In another embodiment, symptoms of Alzheimer's disease may be ameliorated by decreasing the level of an API or API activity by using gene sequences encoding the API in conjunction with well-known gene “knock-out,” ribozyme or triple helix methods to decrease gene expression of an API. In this approach ribozyme or triple helix molecules are used to modulate the activity, expression or synthesis of the gene encoding the API, and thus to ameliorate the symptoms of Alzheimer's disease. Such molecules may be designed to reduce or inhibit expression of a mutant or non-mutant target gene. Techniques for the production and use of such molecules are well known to those of skill in the art.

Ribozyme molecules designed to catalytically cleave gene mRNA transcripts encoding an API can be used to prevent translation of target gene mRNA anid, therefore, expression of the gene product. (See, e.g., PCT International Publication WO90/11364, published Oct. 4, 1990; Sarver et al., 1990, Science 247:1222-1225).

Ribozymes are enzymatic RNA molecules capable of catalyzing the specific cleavage of RNA. (For a review, see Rossi, 1994,Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the-well known catalytic sequence responsible for mRNA cleavage. For this sequence, see, e.g., U.S. Pat. No. 5,093,246, which is incorporated herein by reference in its entirety.

While ribozymes that cleave mRNA at site specific recognition sequences can be used to destroy mRNAs encoding an API, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334, 585-591, each of which is incorporated herein by reference in its entirety.

Preferably the ribozyme is engineered so that the cleavage recognition site is located near the 5′ end of the mRNA encoding the API, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al., 1984, Science, 224, 574-578; Zaug and Cech, 1986, Science, 231, 470475; Zaug, et al., 1986, Nature, 324, 429-433; published International patent application No. WO 88/04300 by University Patents Inc.; Been and Cech, 1986, Cell, 47, 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the gene encoding the API.

As in the antisense approach, the ribozymes can be composed of modified oligonucleotides (e.g., for improved stability, targeting, etc.) and should be delivered to cells that express the API in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA encoding the API and inhibit translation. Because ribozymes, unlike antisense molecules, are catalytic, a lower intracellular concentration is required for efficacy.

Endogenous API expression can also be reduced by inactivating or “knocking out” the gene encoding the API, or the promoter of such a gene, using targeted homologous recombination (e.g., see Smithies, et al., 1985,. Nature 317:230-234; Thomas and Capecchi, 1987, Cell 51:503-512; Thompson et al., 1989, Cell 5:313-321; and Zijlstra et al., 1989, Nature 342:435-438, each of which is incorporated by reference herein in its entirety). For example, a mutant gene encoding a non-functional API (or a completely unrelated DNA sequence) flanked by DNA homologous to the endogenous gene (either the coding regions or regulatory regions of the gene encoding the API) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However, this approach can be adapted for use in humans provided the recombinant DNA constructs are directly administered or targeted to the required site in vivo using appropriate viral vectors.

Alternatively, the endogenous expression of a gene encoding an API can be reduced by targeting deoxyribonucleotide sequences complementary to the regulatory region of the gene (i.e., the gene promoter and/or enhancers) to form triple helical structures that prevent transcription of the gene encoding the API in target cells in the body. (See generally, Helene, 1991, Anticancer Drug Des., 6(6), 569-584; Helene, et al., 1992, Ann. N.Y. Acad. Sci., 660, 27-36; and Maher, 1992, Bioassays 14(12), 807-815).

Nucleic acid molecules to be used in triplex helix formation for the inhibition of transcription in the present invention should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In one embodiment, wherein the antisense, ribozyme, or triple helix molecules described herein are utilized to inhibit mutant gene expression, it is possible that the technique may so efficiently reduce or inhibit the transcription (triple helix) or translation (antisense, ribozyme) of mRNA produced by normal gene alleles of an API that the situation may arise wherein the concentration of API present may be lower than is necessary for a normal phenotype. In such cases, to ensure that substantially normal levels of activity of a gene encoding an API are maintained, gene therapy may be used to introduce into cells nucleic acid molecules that encode and express the API that exhibit normal gene activity and that do not contain sequences susceptible to whatever antisense, ribozyme, or triple helix treatments are being utilized. Alternatively, in instances whereby the gene encodes an extracellular protein, normal API can be co-administered in order to maintain the requisite level of API activity.

Antisense RNA and DNA, ribozyme, and triple helix molecules of the invention may be prepared by any method known in the art for the synthesis of DNA and RNA molecules, as discussed above. These include techniques for chemically synthesizing oligodeoxyri-bonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Assays for Therapeutic or Prophylactic Compounds

The present invention also provides assays for use in discovery of pharmaceutical products in order to identify or verify the efficacy of compounds for treatment or prevention of Alzheimer's disease. Agents can be assayed for their ability to restore AF or API levels in a subject having Alzheimer's disease towards levels found in subjects free from Alzheimer's disease or to produce similar changes in experimental animal models of Alzheimer's disease. Compounds able to restore AF or API levels in a subject having Alzheimer's disease towards levels found in subjects free from Alzheimer's disease or to produce similar changes in experimental animal models of Alzheimer's disease can be used as lead compounds for further drug discovery, or used therapeutically. AF and API expression can be assayed by the Preferred Technology, immunoassays, gel electrophoresis followed by visualization, detection of API activity, or any other method taught herein or known to those skilled in the art. Such assays can be used to screen candidate drugs, in clinical monitoring or in drug development, where abundance of an AF or API can serve as a surrogate marker for clinical disease.

In various embodiments, in vitro assays can be carried out with cells representative of cell types involved in a subject's disorder, to determine if a compound has a desired effect upon such cell types.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used. Examples of animal models of Alzheimer's disease include, but are not-limited to, animals that express human familial Alzheimer's disease (FAD) P-amyloid precursor (APP), animals that overexpress human wild-type APP, animals that overexpress β-amyloid 1-42 (βA), animals that express FAD presenillin-1 (PS-1) (see, e.g., Higgins, LS, 1999, Molecular Medicine Today 5:274-276). Further, animal models for Downs syndrome (e.g. TgSOD1, TgPFKL, TgS100β, TgAPP, TgEts2, TgHMG14, TgMNB, Ts65Dn, and Ts1Cje (see, e.g., Kola et al., 1999, Molecular Medicine Today 5:276-277) can be utilized to test compounds that modulate AF or API levels since the neuropathology exhibited by individuals with Downs syndrome is similar to that of Alzheimer's disease. It is also apparent to the skilled artisan that, based upon the present disclosure, transgenic animals can be produced with “knock-out” mutations of the gene or genes encoding one or more APIs. A “knock-out” mutation of a gene is a mutation that causes the mutated gene to not be expressed, or expressed in an aberrant form or at a low level, such that the activity associated with the gene product is nearly or entirely absent. Preferably, the transgenic animal is a mammal, more preferably, the transgenic animal is a mouse.

In one embodiment, test compounds that modulate the expression of an API are identified in non-human animals (e.g., mice, rats, monkeys, rabbits, and guinea pigs), preferably non-human animal models for Alzheimer's disease or Downs syndrome, expressing the API. In accordance with this embodiment, a test compound or a control compound is administered to the animals, and the effect of the test compound on expression of one or more APIs is determined. A test compound that alters the expression of an API (or a plurality of APIs) can be identified by comparing the level of the selected API or APIs (or mRNA(s) encoding the same) in an animal or group of animals treated with a test compound with the level of the API(s) or mRNA(s) in an animal or group of animals treated with a control compound. Techniques known to those of skill in the art can be used to determine the mRNA and protein levels, for example, in situ hybridization. The animals may or may not be sacrificed to assay the effects of a test compound.

In another embodiment, test compounds that modulate the activity of an API or a biologically active portion thereof are identified in non-human animals (e.g., mice, rats, monkeys, rabbits, and guinea pigs), preferably non-human animal models for Alzheimer's disease or Downs syndrome, expressing the API. In accordance with this embodiment, a test compound or a control compound is administered to the animals, and the effect of a test compound on the activity of an API is determined. A test compound that alters the activity of an API (or a plurality of APIs) can be identified by assaying animals treated with a control compound and animals treated with the test compound. The activity of the API can be assessed by detecting induction of a cellular second messenger of the API (e.g., intracellular Ca2+, diacylglycerol, IP3, etc.), detecting catalytic or enzymatic activity of the API or binding partner thereof, detecting the induction of a reporter gene (e.g., a regulatory element that is responsive to an API of the invention operably linked to a nucleic acid encoding a detectable marker, such as luciferase or green fluorescent protein), or detecting a cellular response (e.g., cellular differentiation or cell proliferation). Techniques known to those of skill in the art can be utilized to detect changes in the activity of an API (see, e.g., U.S. Pat. No. 5,401,639, which is incorporated herein in its entirety by reference).

In yet another embodiment, test compounds that modulate the level or expression of an API (or plurality of APIS) are identified in human subjects having Alzheimer's disease or Downs syndrome, preferably those having mild to severe Alzheimer's disease and most preferably those having mild Alzheimer's disease. In accordance with this embodiment, a test compound or a control compound is administered to the human subject, and the effect of a test compound on API expression is determined by analyzing the expression of the API or the mRNA encoding the same in a biological sample (e.g., CSF, serum, plasma, or urine). A test compound that alters the expression of an API can be identified by comparing the level of the API or mRNA encoding the same in a subject or group of subjects treated with a control compound to that in a subject or group of subjects treated with a-test compound. Alternatively, alterations in the expression of an API can be identified by comparing the level of the API or mRNA encoding the same in a subject or group of subjects before and after the administration of a test compound. Any suitable techniques known to those of skill in the art can be used to obtain the biological sample and analyze the mRNA or protein expression. For example, the Preferred Technology described herein can be used to assess changes in the level of an API.

In another embodiment, test compounds that modulate the activity of an API (or plurality of APIs) are identified in human subjects having Alzheimer's disease or Downs syndrome, preferably those having mild to severe Alzheimer's disease and most preferably those with mild Alzheimer's disease. In this embodiment, a test compound or a control compound is administered to the human subject, and the effect of a test compound on the activity of an API is determined. A test compound that alters the activity of an API can be identified by comparing biological samples from subjects treated with a control compound to samples from subjects treated with the test compound. Alternatively, alterations in the activity of an API can be identified by comparing the activity of an API in a subject or group of subjects before and after the administration of a test compound. The activity of the API can be assessed by detecting in a biological sample (e.g., CSF, serum, plasma, or urine) induction of a cellular signal transduction pathway of the API (e.g., intracellular Ca2+, diacylglycerol, IP3, etc.), catalytic or enzymatic activity of the API or a binding partner thereof, or a cellular response, for example, cellular differentiation, or cell proliferation. Techniques known to those of skill in the art can be used to detect changes in the induction of a second messenger of an API or changes in a cellular response.. For example, RT-PCR can be used to detect changes in the induction of a cellular second messenger.

In a particular embodiment, an agent that changes the level or expression of an API towards levels detected in control subjects (e.g., humans free from Alzheimer's disease) is selected for further testing or therapeutic use. In another preferred embodiment, a test compound that changes the activity of an API towards the activity found in control subjects (e.g., humans free from Alzheimer's disease) is selected for further testing or therapeutic use.

In another embodiment, test compounds that reduce the severity of one or more symptoms associated with Alzheimer's disease are identified in human subjects having Alzheimer's disease or Downs syndrome, preferably subjects having mild to severe Alzheimer's disease and most preferably subjects with mild Alzheimer's disease. In accordance with this embodiment, a test compound or a control compound is administered to the subjects, and the effect of a test compound on one or more symptoms of Alzheimer's disease is determined. A test compound that reduces one or more symptoms can be identified by comparing the subjects treated with a control compound to the subjects treated with the test compound. Techniques known to physicians familiar with Alzheimer's disease can be used to determine whether a test compound reduces one or more symptoms associated with Alzheimer's disease. For example, a test compound that enhances memory or reduces confusion in a subject having Alzheimer's disease will be beneficial for treating subjects having Alzheimer's disease.

In a preferred embodiment, an agent that reduces the severity of one or more symptoms associated with Alzheimer's disease in a human having Alzheimer's disease is selected for further testing or therapeutic use.

Therapeutic and Prophylactic Compositions and Their Use

The invention provides methods of treatment comprising administering to a subject an effective amount of an agent of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into CSF or at the site (or former site) of neurodegeneration or to CNS tissue.

In another embodiment, the compound can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105 ). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other suitable controlled release systems are discussed in the review by Langer (1990, Science 249:1527-1533).

In another embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

The present invention also provides pharmaceutical compositions. Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations arid the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of Alzheimer's disease can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### Example: Identification of Proteins Differentially Expressed in the CSF in Alzheimer's Disease

Using the following exemplary and non-limiting procedure, proteins in CSF samples from (a) 148 subjects having Alzheimer's disease, (b) 60 family members of these Alzheimer's disease subjects, and (c) 32 unrelated controls were separated by isoelectric focusing followed by SDS-PAGE and analyzed. From some subjects, serial samples were taken over time. Parts 6.1.1 to 6.1.9 (inclusive) of the procedure set forth below are hereby designated as the “Reference Protocol”.

Materials and Methods

Sample Preparation

A protein assay (Pierce BCA Cat #23225) was performed on each CSF sample as received. Prior to protein separation, each sample was processed for selective depletion of certain proteins, in order to enhance and simplify protein separation and facilitate analysis by removing proteins that may interfere with or limit analysis of proteins of interest. See International Patent Application No. PCT/GB99/01742, filed Jun. 1, 1999, which is incorporated by reference in its entirety, with particular reference to pages 3 and 6.

Removal of albumin, haptoglobin, transferrin and immunoglobin G (IgG) from CSF (“CSF depletion”) was achieved by an affinity chromatography purification step in which the sample was passed through a series of ‘Hi-Trap’ columns containing immobilized antibodies for selective removal of albumin, haptoglobin and transferrin, and protein G for selective removal of immunoglobin G. Two affinity columns in a tandem assembly were prepared by coupling antibodies to protein G-sepharose contained in Hi-Trap columns (Protein G-Sepharose Hi-Trap columns (1 ml) Pharmacia Cat. No. 17-0404-01). This was done by circulating the following solutions sequentially through the columns: (1) Dulbecco's Phosphate Buffered Saline (Gibco BRL Cat. No. 14190-094); (2) concentrated antibody solution; (3) 200 mM sodium carbonate buffer, pH 8.35; (4) cross-linking solution (200 nimsodium carbonate buffer, pH 8.35, 20 mM dimethylpimelimidate); and (5) 500 mM ethanolamine, 500 mM NaCl. A third (un-derivatised) protein G Hi-Trap column was then attached to the lower end of the tandem column assembly.

The chromatographic procedure was automated using an Akta Fast Protein Liquid Chromatography (FPLC) System such that a series of up to seven runs could be performed sequentially. The samples were passed through the series of 3 Hi-Trap columns in which the affinity chromatography media selectively bind the above proteins thereby removing them from the sample. Fractions (typically 3 ml per tube) were collected of unbound material (“Flowthrough fractions”) that eluted through the column during column loading and washing stages and of bound proteins (“Bound/Eluted fractions”) that were eluted by step elution with Immunopure Gentle Ag/Ab Elution Buffer (Pierce Cat. No. 21013). The eluate containing unbound material was collected in fractions which were pooled, desalted/concentrated by centrifugal ultrafiltration and stored to await further analysis by 2D PAGE.

A volume of depleted CSF containing approximately 300 μg of total protein was aliquoted and an equal volume of 10% (w/v) SDS (Fluka 71729), 2.3% (w/v) dithiothreitol (BDH 443852A) was added. The sample was heated at 95° C. for 5 mins, and then allowed to cool to 20° C. 125 μl of the following buffer was then added to the sample:

8M urea (3DH 452043w)

4% CHAPS (Sigma C3023)

65 mM dithiotheitol (DTT)

2% (v/v) Resolytes 3.5-10 (BDH 44338 2x)

This mixture was vortexed, and centrifuged at 13000 rpm for 5 mins at 15° C., and the supernatant was analyzed by isoelectric focusing.

Isoelectric Focusing

Isoelectric focusing (IEF), was performed using the Immobiline® DryStrip Kit (Pharmacia BioTech), following the procedure described in the manufacturer's instructions, see Instructions for Immobiline® DryStrip Kit, Pharmacia, # 18-1038-63, Edition AB (incorporated herein by reference in its entirety). Immobilized pH Gradient (IPG) strips (18 cm, pH 3-10 non-linear strips; Pharmacia Cat. # 17-1235-01) were rehydrated overnight at 20° C. in a solution of 8M urea, 2% (w/v) CHAPS, 10 mM DTT, 2% (v/v) Resolytes 3.5-10, as described in the Immobiline DryStrip Users Manual. For IEF, 50 μl of supernatant (prepared as above) was loaded onto a strip, with the cup-loading units being placed at the basic end of the strip. The loaded gels were then covered with mineral oil (Pharmacia 17-3335-01) and a voltage was immediately applied to the strips according to the following profile, using a Pharmacia EPS3500XL power supply (Cat 19-3500-01):

Initial voltage 300V for 2 hrs

Linear Ramp from 300V to 3500V over 3 hrs

Hold at 3500V for 19 hrs

For all stages of the process, the current limit was set to 10 mA for 12 gels, and the wattage limit to 5 W. The temperature was held at 20° C. throughout the run.

Gel Equilibration and SDS-PAGE

After the final 19 hr step, the strips were immediately removed and immersed for 10 mins at 20° C. in a first solution of the following composition: 6M urea; 2% (w/v) DTT; 2% (w/v) SDS; 30% (v/v) glycerol (Fluka 49767); 0.05M Tris/HCl, pH 6.8 (Sigma Cat T-1503). The strips were removed from the first solution and immersed for 10 mins at 20° C. in a second solution of the following composition: 6M urea; 2% (w/v) iodoacetamide (Sigma I-6125); 2% (w/v) SDS; 30% (v/v) glycerol; 0.05M Tris/HCl, pH 6.8. After removal from the second solution, the strips were loaded onto supported gels for SDS-PAGE according to Hochstrasser et al., 1988, Analytical Biochemistry 173: 412-423 (incorporated herein by reference in its entirety), with modifications as specified below.

Preparation of Supported Gels

The gels were cast between two glass plates of the following dimensions: 23 cm wide×24 cm long (back plate); 23 cm wide×24 cm long with a 2 cm deep notch in the central 19 cm (front plate). To promote covalent attachment of SDS-PAGE gels, the back plate was treated with a 0.4% solution of γ-methacryl-oxypropyltrimethoxysilane in ethanol (BindSilane; Pharmacia Cat. # 17-1330-01). The front plate was treated with (RepelSilane™ Pharmacia Cat. # 17-1332-01) to reduce adhesion of the gel. Excess reagent was removed by washing with water, and the plates were allowed to dry. At this stage, both as identification for the gel, and as a marker to identify the coated face of-the plate, an adhesive bar-code was attached to the back plate in a position such that it would not come into contact with the gel matrix.

The dried plates were assembled into a casing box with a capacity of 13 gel sandwiches. The top and bottom plates of each sandwich were spaced by means of 1 mm thick spacers, 2.5 cm wide. The sandwiches were interleaved with acetate sheets to facilitate separation of the sandwiches after gel polymerization. Casting was then carried out according to Hochstrasser et al., op. cit.

A 9-16% linear polyacrylamide gradient was cast, extending up to a point 2 cm below the level of the notch in the front plate, using the Angelique gradient casting system (Large Scale Biology). Stock solutions were as follows. Acrylamide (40% in water) was from Serva (Cat. # 10677). The cross-linking agent was PDA (BioRad 161-0202), at a concentration of 2.6% (w/w) of the total starting monomer content. The gel buffer was 0.375M Tris/HCl, pH 8.8. The polymerization catalyst was 0.05% (v/v) TEMED (BioRad 161-0801), and the initiator was 0.1% (w/v) APS (BioRad 161-0700). No SDS was included in the gel and no stacking gel was used. The cast gels were allowed to polymerize at 20° C. overnight, and then stored at 4° C. in sealed polyethylene bags with 6 ml of gel buffer, and were used within 4 weeks.

SDS-PAGE

A solution of 0.5% (w/v) agarose (Fluka Cat 05075) was prepared in running buffer (0.025M Tris, 0.198M glycine (Fluka 50050), 1% (w/v) SDS, supplemented by a trace of bromophenol blue). The agarose suspension was heated to 70° C. with stirring, until the agarose had dissolved. The top of the supported 2nd D gel was filled with the agarose solution, and the equilibrated strip was placed into the agarose, and tapped gently with a palette knife until the gel was intimately in contact with the 2nd D gel. The gels were placed in the 2nd D running tank, as described by Amess et al., 1995, Electrophoresis 16: 1255-1267 (incorporated herein by reference in its entirety). The tank was filled with running buffer (as above) until the level of the buffer was just higher than the top of the region of the 2nd D gels which contained polyacrylamide, so as to achieve efficient cooling of the active gel area. Running buffer was added to the top buffer compartments formed by the gels, and then voltage was applied immediately to the gels using a Consort E-833 power supply. For 1 hour, the gels were run at 20 mA/gel. The wattage limit was set to 150W for a tank containing 6 gels, and the voltage limit was set to 600V. After 1 hour, the gels were then run at 40 mA/gel, with the same voltage and wattage limits as before, until the bromophenol blue line was 0.5 cm from the bottom of the gel. The temperature of the buffer was held at 16° C. throughout the run. Gels were not run in duplicate.

Staining

Upon completion of the electrbphoresis run, the gels were immediately removed from the tank for fixation. The top plate of the gel cassette was carefully removed, leaving the gel bonded to the bottom plate. The bottom plate with its attached gel was then placed into a staining apparatus, which can accommodate 12 gels. The gels were completely immersed in fixative solution of 40% (v/v) ethanol (BDH 28719), 10% (v/v) acetic acid (BDH 100016X), 50% (v/v) water (MilliQ-Millipore), which was continuously circulated over the gels. After an overnight incubation, the fixative was drained from the tank, and the gels were primed by immersion in 7.5% (v/v) acetic acid, 0.05% (w/v) SDS, 92.5% (v/v) water for 30 mins. The priming solution was then drained, and the gels were stained by complete immersion for 4 hours in a staining solution of Pyridinium, 4-[2-[4-(dipentylamino)-2-trifluoromethylphenyl]ethenyl]-1-(sulfobutyl)-, inner salt, prepared by diluting a stock solution of this dye (2 mg/ml in DMSO) in 7.5% (v/v) aqueous acetic acid to give a final concentration of 1.2 mg/l; the staining solution was vacuum filtered through a 0.4 μm filter (Duropore) before use.

Imaging of the Gel

A computer-readable output was produced by imaging the fluorescently stained gels with the Apollo 2 scanner (Oxford Glycosciences, Oxford, UK) described in section 5.1, supra. This scanner has a gel carrier with four integral fluorescent markers (Designated M1, M2, M3, M4) that are used to correct the image geometry and are a quality control feature to confirm that the scanning has been performed correctly.

For scanning, the gels were removed from the stain, rinsed with water and allowed to air dry briefly, and imaged on the Apollo 2. After imaging, the gels were sealed in polyethylene bags containing a small volume of staining solution, and then stored at 4° C.

Digital Analysis of the Data

The data were processed as described in U.S. application Ser. No. 08/980,574, (published as WO 98/23950) at Sections 5.4 and 5.5 (incorporated herein by reference), as set forth more particularly below.

The output from the scanner was first processed using the MELANIE® II 2D PAGE analysis program (Release 2.2, 1997, BioRad Laboratories, Hercules, Calif., Cat. # 170-7566) to autodetect the registration points, M1, M2, M3 and M4; to autocrop the images (i.e., to eliminate signals originating from areas of the scanned image lying outside the boundaries of the gel, e.g. the reference frame); to filter out artifacts due to dust; to detect and quantify features; and to create image files in GIF format. Features were detected using the following parameters:

Smooths=2

Laplacian threshold 50

Partials threshold 1

Saturation=100

Peakedness=0

Minimum Perimeter=10

6.1.9 Assignment of pI and MW Values

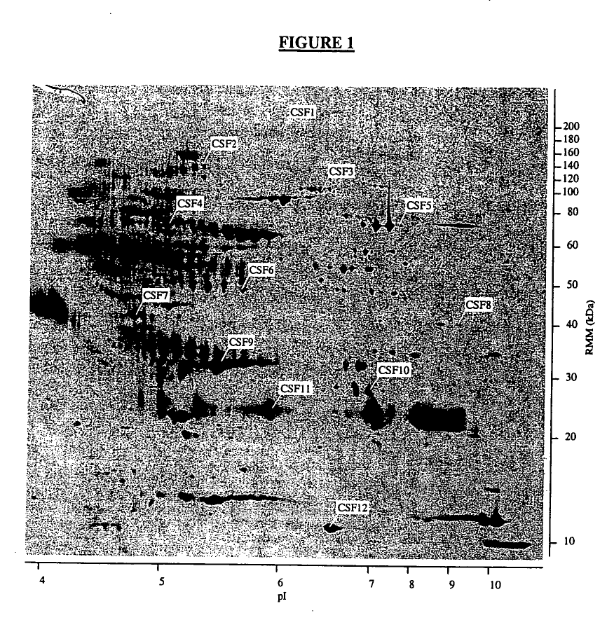

Landmark identification was used to determine the pI and MW of features detected in the images. Twelve landmark features, designated CSF1 to CSF12, were identified in a standard CSF image obtained from a pooled sample. These landmark features are identified in FIG. 1 and were assigned the pI and/or MW values identified in Table XI.

TABLE XI
Landmark Features Used In This Study

| | | MW |
|---|---|---|
| Name | pl | (Da) |
| CSF1 | 5.96 | 185230 |
| CSF2 | 5.39 | 141700 |
| CSF3 | 6.29 | 100730 |
| CSF4 | 5.06 | 71270 |
| CSF5 | 7.68 | 68370 |
| CSF6 | 5.67 | 48090 |
| CSF7 | 4.78 | 41340 |
| CSF8 | 9.2 | 40000 |
| CSF9 | 5.5 | 31900 |
| CSF10 | 6.94 | 27440 |
| CSF11 | 5.9 | 23990 |
| CSF12 | 6.43 | 10960 |

As many of these landmarks as possible were identified in each gel image of the dataset. Each feature in the study gels was then assigned a pI value by linear interpolation or extrapolation (using the MELANIE®-II software) to the two nearest landmarks, and was assigned a MW value by linear interpolation or extrapolation (using the MELANIE®-II software) to the two nearest landmarks.

Matching With Primary Master Image

Images were edited to remove gross artifacts such as dust, to reject images which had gross abnormalities such as smearing of protein features, or were of too low a loading or overall image intensity to allow identification of more than the most intense features, or were of too poor a resolution to allow accurate detection of features. Images were then compared by pairing with one common image from the whole sample set. This common image, the “primary master image”, was selected on the basis of protein load (maximum load consistent with maximum feature detection), a well resolved myoglobin region, (myoglobin was used as an internal standard), and general image quality. Additionally, the primary master image was chosen to be an image which appeared to be generally representative of all those to be included in the analysis. (This process by which a primary master gel was judged to be representative of the study gels was rechecked by the method described below and in the event that the primary master gel was seen to be unrepresentative, it was rejected and the process repeated until a representative primary master gel was found.)

Each of the remaining study gel images was individually matched to the primary master image such that common protein features were paired between the primary master image and each individual study gel image as described below.

6.1.1 Cross-Matching Between Samples

To facilitate statistical analysis of large numbers of samples for purposes of identifying features that are differentially expressed, the geometry of each study gel was adjusted for maximum alignment between its pattern of protein features, and that of the primary master, as follows. Each of the study gel images was individually transformed into the geometry of the primary master image using a multi-resolution warping procedure. This procedure corrects the image geometry for the distortions brought about by small changes in the physical parameters of the electrophoresis separation process from one sample to another. The observed changes are such that the distortions found are not simple geometric distortions, but rather a smooth flow, with variations at both local and global scale.

The fundamental principle in multi-resolution modeling is that smooth signals may be modeled as an evolution through ‘scale space’, in which details at successively finer scales are added to a low resolution approximation to obtain the high resolution signal. This type of model is applied to the flow field of vectors (defined at each pixel position on the reference image) and allows flows of arbitrary smoothness to be modeled with relatively few degrees of freedom. Each image is first reduced to a stack, or pyramid, of images derived from the initial image, but smoothed and reduced in resolution by a factor of 2 in each direction at every level (Gaussian pyramid) and a corresponding difference image is also computed at each level, representing the difference between the smoothed image and its progenitor (Lapiacian pyramid). Thus the Laplacian images represent the details in the image at different scales.

To estimate the distortion between any 2 given images, a calculation was performed at level 7 in the pyramid. (i.e. after 7 successive reductions in resolution). The Laplacian images were segmented into a grid of 16×16 pixels, with 50% overlap between adjacent grid positions in both directions, and the cross correlation between corresponding grid squares on the reference and the test images was computed. The distortion displacement was then given by the location of the maximum in the correlation matrix. After all displacements had been calculated at a particular level, they were interpolated to the next level in the pyramid, applied to the test image, and then further corrections to the displacements were calculated at the next scale.

The warping process brought about good alignment between the common features in the primary master image, and the images for the other samples. The MELANIE® II 2D PAGE analysis program was used to calculate and record approximately 500-700 matched feature pairs between the primary master and each of the other images. The accuracy of this program was significantly enhanced by the alignment of the images in the manner described above. To improve accuracy still further, all pairings were finally examined by eye in the MelView interactive editing program and residual recognizably incorrect pairings were removed. Where the number of such recognizably incorrect pairings exceeded the overall reproducibility of the Preferred Technology (as measured by repeat analysis of the same biological sample) the gel selected to be the primary master gel was judged to be insufficiently representative of the study gels to serve as a primary master gel. In that case, the gel chosen as the primary master gel was rejected, and different gel was selected as the primary master gel, and the process was repeated.

All the images were then added together to create a composite master image, and the positions and shapes of all the gel features of all the component images were super-imposed onto this composite master as described below.

Once all the initial pairs had been computed, corrected and saved, a second pass was performed whereby the original (unwarped) images were transformed a second time to the geometry of the primary master, this time using a flow field computed by smooth interpolation of the multiple tie-points defined by the centroids of the paired gel features. A composite master image was thus generated by initialising the primary master image with its feature descriptors. As each image was transformed into the primary master geometry, it was digitally summed pixel by pixel into the composite master image, and the features that had not been paired by the procedure outlined above were likewise added to the composite master image description, with their centroids adjusted to the master geometry using the flow field correction.

The final stage of processing was applied to the composite master image and its feature descriptors, which now represent all the features from all the images in the study transformed to a common geometry. The features were grouped together into linked sets or “clusters”, according to the degree of overlap between them. Each cluster was then given a unique identifying index, the molecular cluster index (MCI).

An MCI identifies a set of matched features on different images. Thus an MCI represents a protein or proteins eluting at equivalent positions in the 2D separation in different samples.

Construction of Profiles

After matching all component gels in the study to the final composite master image, the intensity of each feature was measured and stored. The end result of this analysis was the generation of a digital profile which contained, for each identified feature: 1) a unique identification code relative to corresponding feature within the composite master image (MCI), 2) the x, y coordinates of the features within the gel, 3) the isoelectric point (pI) of the AFs, 4) the apparent molecular weight (MW) of the AFs, 5) the signal value, 6) the standard deviation for each of the preceding measurements, and 7) a method of linking the MCI of each feature to the master gel to which this feature was matched. By virtue of a Laboratory Information Management System (LIMS), this MCI profile was traceable to the actual stored gel from which it was generated, so that proteins identified by computer analysis of gel profile databases could be retrieved. The LIMS also permitted the profile to bet traced back to an original sample or patient.

Differental Analysis of the Profiles

For the pooled gel data within each sample set (Alzheimer's CSF and normal CSF), the profiles were analyzed to identify and select those features differentially present in the profiles. These selected features were then assembled into an Alzheimer's pooled gel feature set. Matching features of each feature set were then compared to identify those features showing at least a 2-fold difference in mean intensity between Alzheimer's CSF and normal CSF. Differentially present features were identified as Alzheimer's Disease Associated Features (AFs).

Statistical Analysis of the Profiles

The MCI data was represented in statistical models in two forms: 1) percent of total protein volume for a given gel (PCTVOL) and 2) absolute volume, scaled by the total volume loaded on the gel (VOL). A value of 0 was entered for PCTVOL and VOL if an MCI did not appear on a particular gel. For most analyzes, in order for an MCI to be considered in the statistical model, it had to have non-zero values for PCTVOL and VOL in at least 75% of gels in at least one of the diagnosis groups in the analysis (described below).

The complementary statistical strategies specified below were used to identify AFs from the MCIs within the mastergroup.

(I) Group Analysis

The purpose of these analyses was to characterize differences among gels from individuals with different clinical diagnoses. The diagnosis groups were 1) autopsy-confirmed (AD) vs. normal controls (NCO) at their first sample, 2) Dementia Alzheimer's type (DAT) with an initial sample within 3 years of disease onset vs. NCO, and 3) last sample of first-degree relatives of individuals diagnosed with dementia of Alzheimer's type without a clinical diagnosis of dementia (NCF) vs. NCO. The following statistical techniques were used in the group analyses:

(1) Linear model

A linear model controlling for age and gender that compared a DAT group vs the NCO group with regard to the rank of the volume.

(2) Classification Trees

Classification trees were used with the MCI volumes as predictors, and clinical diagnosis as the response. The algorithm looks for ‘split points’ in the predictors that partition the data into homogeneous sets according to the response variable. After evaluating all possible splits for a given node of the tree, the split is chosen that maximizes the change in deviance according to a multinomial likelihood model. Tree models were fit to both the original data and data from bootstrap samples of the original data (sampling with replacement). The statistical test involved whether a given MCI proved to be an important ‘split point’ to determine diagnosis, either in the original data tree or a bootstrap sample tree.

(3) Logistic Regression Model

A logistic regression model was used to model the probability of being AD. The volumes of the various MCI's were used as the explanatory variables. A stepwise procedure was used to select 5 MCI's.

Criteria for inclusion based on the group analyses:

Information from all of the above described analyses were used to select MCI's that:

1. Were among the 5-6 MCI's with the smallest p-value for a given analysis

2. Appeared in the smallest 100 p-values for 2 or more analysis

3. Appeared as an important split-point in a classification tree

4. Had desired distributional properties

(II) Longitudinal Analysis

The purpose of the longitudinal analyses was to identify AF's associated with changes in disease state as measured by the MMSEM, a combination of the MMSE, CDR, and GDS assessment measures. DAT subjects with two or more samples were used in these analyses.

There were two models employed in the longitudinal analyses. In the first, the goal was to identify AF's for which changes in volume were significantly correlated with changes in the MMSEM. For each AF, MMSEM was regressed on the rank of the volume after controlling for age and subject. AF's with p-values less than 0.05, in the top 100 of any of the group analyses, and consistent with the group analyses in terms of up or down regulation were included.

The goal of the second model was to identify AF's for which volume in a subject's first sample was a significant predictor of disease progression rate during the period following the time of the first sample. First, a simple linear regression model was used to estimate a progression rate based on the MMSEM for each subject. Only subjects with an initial MMSEM greater than or equal to 12 and with greater than four months between the first and last samples were used. In addition, only samples within the first three years of the first sample were used. Regression modeling and split-sample validation were then used to identify significant AF's. More specifically, subjects were first randomly divided into two groups. For each group, stepwise weighted least-squares (WLS) regression using the rank of volume from each subject's first sample was used to select the five best AF's for predicting progression rate. If an AF was in the top five in one group and yielded a slope estimate with the same sign when included in the other group, it was included. In addition, the top five AF's from a stepwise WLS on both groups combined were included.

Recovery and Analysis of Selected Proteins

Proteins in AFs were robotically excised and processed to generate tryptic digest peptides. Tryptic peptides were analyzed by mass spectrometry using a PerSeptive Biosystems Voyager- DETM STR Matrix-Assisted Laser Desorption Ionization Time-of-Flight (MALDI-TOF) mass spectrometer, and selected tryptic peptides were analyzed by tandem mass spectrometry (MS/MS) using a Micromass Quadrupole Time-of-Flight (Q-TOF) mass spectrometer (Micromass, Altrincham, U.K.) equipped with a nanoflow™ electrospray Z-spray source. For partial amino acid sequencing and identification of APIs uninterpreted tandem mass spectra of tryptic peptides were searched using the SEQUEST search program (Eng et al., 1994, J. Am. Soc. Mass Spectrom. 5:976-989), version v.C.1. Criteria for database identification included: the cleavage specificity of trypsin; the detection of a suite of a, b and y ions in peptides returned from the database, and a mass increment for all Cys residues to account for carbamidomethylation. The database searched was database constructed of protein entries in the non-redundant database held by the National Centre for Biotechnology Information (NCBI) which is accessible at http://www.ncbi.nlm.nih.gov/. Following identification of proteins through spectral-spectral correlation using the SEQUEST program, masses detected in MALDI-TOF mass spectra were assigned to tryptic digest peptides within the proteins identified. In cases where no proteins could be identified through searching with unint.erpreted MS/MS spectra of tryptic digest peptides using the SE QUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662).

Results

These initial experiments identified 117 features that were decreased and 64 features that were increased in Alzheimer's disease CSF as compared with normal CSF. Details of these AFs are provided in Tables I and II. Each AF was differentially present in Alzheimer's disease CSF as compared with normal CSF. For some preferred AFs (AF-1, AF-2, AF-3., AF-4, AF-5, AF-6, AF-7, AF-8, AF-9, AF-10, AF-13, AF-14, AF-15, AF-16, AF-17, AF-19, AF-10, AF-21,-AF-23, AF-24, AF-25, AF-26, AF-28, AF-29, AF-30, AF-32, AF-33, AF-35, AF-37, AF-38,.AF-39, AF-40, AF-42, AF43, AF-46, AF-47, AF-48, AF-51, AF-54, AF-55, AF-56, AF-57, AF-59, AF-60, AF-62, AF-64, AF-65, AF-66, AF-67, AF-68, AF-69, AF-71, AF-73, AF-75, AF-76, AF-149, AF-150, AF-152, AF-153, AF-154, AF-155, AF-156, AF-157, AF-159, AF-160, AF-161, AF-162, AF-163, AF-165, AF-166, AF-167, AF-168, AF-169, AF-170, AF-171, AF-173, AF-174, AF-177, AF-180, AF-181, AF-182, AF-183, AF-184, AF-185, AF-186, AF-187, AF-188, AF-189, AF-190, AF-191, AF-192) the difference was highly significant (p<0.01), and for certain highly preferred AFs (AF-2, AF-3, AF-5, AF-6, AF-9, AF-10, AF-13, AF-15, AF-16, AF-17, AF-20, AF-21, AF-23, AF-24, AF-28, AF-29, AF-30, AF-33, AF-37, AF-52, AF-55, AF-57, AF-62, AF-64, AF-66, AF-73, AF-150, AF-154, AF-155, AF-159, AF-161, AF-165, AF-166, AF-168, AF-169, AF-183, AF-187, AF-189, AF-190, AF-191, AF-192), the difference was still more significant (p<0.001).

Partial amino acid sequences were determined for the differentially present APIs in these AFs. Details of these APIs are provided in Tables IV and V. Computer searches of public databases identified at least one API for which neither the partial amino acid sequence, nor any oligonucleotide encoding such a peptide sequence, was described in any public database examined.

### Example: Diagnosis and Treatment of Alzheimer's Disease

The following example illustrate the use of an API of the invention for screening, treatment or diagnosis of Alzheimer's disease. The following example also illustrates the use of modulators (e.g., agonist or antagonists) of an API of the invention to treat or prevent Alzheimer's disease.

Pigment epithelium-derived factor (PEDF) is a neurotrophic protein synthesized and secreted by retinal pigment epithelial cells in early embryogenesis and has been shown to be present in the extracellular matrix between the RPE cells and the neural retina. It induces neuronal differentiation and promotes survival of neurons of the central nervous system from degeneration caused by serum withdrawal or glutamate cytotoxicity. PEDF has been shown to protect immature but not mature cerebellar cells from apoptotic death, acting as a survival factor for such cells, as well as protecting them against glutamate and hydrogen peroxide toxicity. PEDF binds to glycosaminoglycans and to an 80 kDa receptor present on the surface of retinoblastoma and cerebellar granule cells. PEDF binding to the 80 kDa receptor, as well as PEDF activity, may be blocked by antibodies recognizing PEDF, and by a 44 amino acid fragment (amino acids 78-121) of PEDF.

The expression of an isoform of PEDF with a molecular weight of 33,401 kDa and pI of 6.74 has been shown herein to be significantly increased in the cerebrospinal fluid (CSF) of subjects having Alzheimer's disease as compared with the CSF of subjects free from Alzheimer's disease. Thus, quantitative detection of PEDF in CSF can be used to diagnose Alzheimer's disease, determine the progression of Alzheimer's disease or monitor the effectiveness of a therapy for Alzheimer's disease.

In one embodiment of the invention, compounds that modulate (i.e., upregulate or downregulate) the expression, activity or both the expression and activity of PEDF are administered to a subject in need of treatment or for prophylaxis of Alzheimer's disease. Antibodies that modulate the expression, activity or both the expression and activity of PEDF are suitable for this purpose. In addition, nucleic acids coding for all or a portion of PEDF, or nucleic acids complementary to all or a portion of PEDF, may be administered. PEDF, or fragments of the PEDF polypeptide may also be administered.

The invention also provides screening assays to identify additional compounds that modulate the expression of PEDF or activity of PEDF. Compounds that modulate the expression of PEDF in vitro can be identified by comparing the expression of PEDF in cells treated with a test compound to the expression of PEDF in cells treated with a control compound (e.g., saline).. Methods for detecting expression of PEDF are known in the art and include measuring the level of PEDF RNA (e.g.; by northern blot analysis or RT-PCR) and measuring PEDF protein (e.g., by immunoassay or western blot analysis). Compounds that modulate the activity of PEDF can be identified by comparing the ability of a test compound to agonize or antagonize a function of PEDF, such as its neurotrophic activity or its binding to the 80 kDa receptor, to the ability of a control compound (e.g., saline) to inhibit the same function of PEDF. Compounds capable of modulating PEDF binding to its receptor or PEDF activity are identified as compounds suitable for further development as a compound useful for the treatment of Alzheimer's disease.

Binding between PEDF and its receptor can be determined by, for example, contacting PEDF with cells known to express the PEDF receptor and assaying the extent of binding between PEDF and the cell surface receptor, or by contacting PEDF with its receptor in a cell-free assay, i.e., an assay where the PEDF and PEDF receptor are isolated, and, preferably, recombinantly produced, and assaying the extent of binding between PEDF and its receptor. Through the use of such assays, candidate compounds may be tested for their ability to agonize or antagonize the binding of PEDF to its receptor.

Compounds identified in vitro that affect the expression or activity of PEDF can be tested in vivo in animal models of Alzheimer's disease or Downs syndrome, or in subjects having Alzheimer's disease, to determine their therapeutic efficacy.

The present invention is not to be limited in terms of the particular embodiments described in this application, which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The contents of each reference, patent and patent application cited in this application is hereby incorporated by reference in its entirety.

1. A method for screening, diagnosis or prognosis of Alzheimer's disease in a mammal, for identifying a mammal at risk of developing Alzheimer's disease, and/or for monitoring the effect of therapy administered to a mammal having Alzheimer's disease, said method comprising: (a) analyzing a test sample of cerebrospinal fluid from the mammal by two dimensional electrophoresis to generate a two-dimensional array of one or more of the following Alzheimer's Disease-Associated Features (AFs): AF-1, AF-2, AF-3, AF-4, AF-5, AF-6, AF-7, AF-8, AF-9, AF-10, AF-13, AF-14, AF-15, AF-16, AF-17, AF-18, AF-19, AF-20, AF-21, AF-22, AF-23, AF-24, AF-25, AF-26, AF-27, AF-28, AF-29, AF-30, AF-31, AF-32, AF-33, AF-34, AF-35, AF-36, AF-37, AF-38, AF-39, AF-40, AF-41, AF-42, AF-43, AF-44, AF-45, AF-46, AF-47, AF-48, AF-49, AF-50, AF-51, AF-52, AF-53, AF-54, AF-55, AF-56, AF-57, AF-58, AF-59, AF-60, AF-61, AF-62, AF-63, AF-64, AF-65, AF-66, AF-67, AF-68, AF-69, AF-70, AF-71, AF-72, AF-73, AF-74, AF-75, AF-76, AF-77, AF-78, AF-79, AF-80, AF-81, AF-82, AF-83, AF-84, AF-85, AF-86, AF-87, AF-88, AF-89, AF-90, AF-91, AF-92, AF-93, AF-94, AF-95, AF-96, AF-98, AF-99, AF-100, AF-101, AF-102, AF-103, AF-104, AF-105, AF-107, AF-108, AF-110, AF-111, AF-112, AF-114, AF-115, AF-116, AF-117, AF-118, AF-119, AF-121, AF-122, AF-123, AF-124, AF-125, AF-126, AF-127, AF-128, AF-129, AF-130, AF-131, AF-132, AF-133, AF-134, AF-137, AF-139, AF-140, AF-141, AF-142, AF-143, AF-144, AF-145, AF-146, AF-147, AF-148, AF-149, AF-150, AF-151, AF-152, AF-153, AF-154, AF-155, AF-156, AF-157, AF-159, AF-160, AF-161, AF-162, AF-163, AF-164, AF-165, AF-166, AF-167, AF-168, AF-169, AF-170, AF-171, AF-172, AF-173, AF-174, AF-175, AF-176, AF-177, AF-178, AF-179, AF-180, AF-181, AF-182, AF-183, AF-184, AF-185, AF-186, AF-187, AF-188, AF-189, AF-190, AF-191, or AF-191; and (b) comparing the abundance of each chosen feature in the test sample with the abundance of that chosen feature in body fluid from one or more persons free from Alzheimer's disease, or with a previously determined reference range for that feature in subjects free from Alzheimer's disease, or with the abundance at least one Expression Reference Feature (ERF) in the test sample.

2. The method of claim 1, wherein said method is for screening or diagnosis of Alzheimer's disease and the relative abundance of at least one chosen feature correlates with the presence or absence of Alzheimer's disease.

3. The method of claim 1, wherein said method is for monitoring the effect of therapy administered to a subject having Alzheimer's disease and the relative abundance of at least one chosen feature correlates with the severity of Alzheimer's disease.

4. A method for screening, diagnosis or prognosis of Alzheimer's disease in a mammal for identifying a mammal at risk of developing Alzheimer's disease, or for monitoring the effect of therapy administered to a mammal having Alzheimer's disease, said method comprising; (a) quantitatively detecting, in a sample of cerebrospinal fluid from the mammal, at least one of the following Alzheimer's Disease-Associated Protein Isoforms (APIs): API-1, API-2, API-3, API-4, API-5, API-6, API-7, API-8, API-9, API-10, API-14, API-15, API-16, API-17, API-18, API-19, API-20, API-22, API-23, API-24, API-25, API-26, API-27, API-28, API-30, API-33, API-34, API-35, API-36, API-37, API-38, API-39, API-40, API-41, API-42, API-43, API-44, API-45, API-46, API-47, API-48, API-49, API-50, API-51, API-52, API-53, API-54, API-55, API-56, API-57, API-58, API-59, API-60, API-61, API-62, API-63, API-64, API-65, API-66, API-67, API-68, API-69, API-70, API-71, API-72, API-73, API-74, API-75, API-76, API-77, API-78, API-79, API-80, API-81, API-82, API-83, API-84, API-85, API-86, API-88, API-89, API-90, API-91, API-92, API-93, API-95, API-97, API-98, API-99, API-101, API-102, API-103, API-104, API-107, API-108, API-111, API-112, API-113, API-114, API-116, API-118, API-119, API-120, API-121, API-122, API-123, API-124, API-125, API-126, API-127, API-128, API-130, API-131, API-132, API-134, API-135, API-136, API-137, API-138, API-139, API-140, API-141, API-142, API-143, API-144, API-145, API-146, API-147, API-148, API-149, API-150, API-151, API-152, API-153, API-155, API-158, API-159, API-160, API-161, API-162, API-163, API-165, API-166, API-167, API-168, API-169, API-170, API-171, API-172, API-173, API-174, API-175, API-176, API-177, API-178, API-179, API-180, API-181, API-182, API-183, API-184, API-185, API-186, API-187, API-188, API-189, API-190, API-191, API-192, API-194, API-196, API-197, API-198, API-199, API-200, API-201, API-202, API-210, API-214, API-215, API-217, API-219, API-220, API-221, API-222, API-223, API-224, API-225, API-232, API-233, API-234, API-237, API-238, API-239, API-240, API-241, API-242, API-243, API-244, API-245, API-246, API-247, or API-248; and (b) comparing the level or amount of said isoform or isoforms detected in step (a) with a control.

5. The method according to claim 4, wherein the step of quantitatively detecting comprises testing at least one aliquot of the sample, said testing comprising: (a) contacting the aliquot with an antibody that is immunospecific for a preselected API; (b) quantitatively measuring any binding that has occurred between the antibody and at least one species in the aliquot; and (c) comparing the results of step (b) to a control.

6. The method according to claim 5, wherein the antibody is a monoclonal antibody.

7. The method according to claim 5, wherein the antibody is chimeric.

8. The method according to claim 5, wherein the step of quantitatively detecting comprises testing a plurality of aliquots with a plurality of antibodies for quantitative detection of a plurality of preselected APIs.

9. The method according to claim 8, wherein the antibodies are monoclonal antibodies.

10. The method according to claim 8, wherein the antibodies are chimeric.

11. A preparation comprising at least one of the following isolated Alzheimer's Disease-Associated Protein Isoform (API) said API selected from API-1, API-2, API-3, API-4, API-5, API-6, API-7, API-8, API-9, API-10, API-14, API-15, API-16, API-17, API-18, API-19, API-20, API-22, API-23, API-24, API-25, API-26, API-27, API-28, API-30, API-33, API-34, API-35, API-36, API-37, API-38, API-39, API-40, API-41, API-42, API-43, API-44, API-45, API-46, API-47, API-48, API-49, API-50, API-51, API-52, API-53, API-54, API-55, API-56, API-57, API-58, API-59, API-60, API-61, API-62, API-63, API-64, API-65, API-66, API-67, API-68, API-69, API-70, API-71, API-72, API-73, API-74, API-75, API-76, API-77, API-78, API-79, API-80, API-81, API-82, API-83, API-84, API-85, API-86, API-88, API-89, API-90, API-91, API-92, API-93, API-95, API-97, API-98, API-99, API-101, API-102, API-103, API-104, API-107, API-108, API-111, API-112, ASPI-113, API-114, API-116, API-118, API-119, API-120, API-121, API-122, API-123, API-124, API-125, API-126, API-127, API-128, API-130, API-131, API-132, API-134, API-135, API-136, API-137, API-138, API-139, API-140, API-141, API-142, API-143, API-144, API-145, API-146, API-147, API-148, API-149, API-150, API-151, API-152, API-153, API-155, API-158, API-159, API-160, API-161, API-162, API-163, API-165, API-166, API-167, API-168, API-169, API-170, API-171, API-172, API-173, API-174, API-175, API-176, API-177, API-178, API-179, API-180, API-181, API-182, API-183, API-184, API-185, API-186, API-187, API-188, API-189, API-190, API-191, API-192, API-194, API-196, API-197, API-198, API-199, API-200, API-201, API-202, API-210, API-214, API-215, API-217, API-219, API-220, API-221, API-222, API-223, API-224, API-225, API-232, API-233, API-234, API-237, API-238, API-239, API-240, API-241, API-242, API-243, API-244, API-245, API-246, API-247, or API-248.

12. A kit comprising the preparation of claim 11, other reagents, and directions for use.

13. The kit of claim 12 comprising a plurality of said preparations.

14. A preparation comprising an isolated human protein, said protein comprising a tryptic digest peptide having the following partial sequence as determined by mass spectrometry: PGLGM.

15. A preparation comprising an isolated human protein, said protein comprising a tryptic digest peptide having the following partial sequence as determined by mass spectroscopy: GPLGM.

16. A preparation comprising an isolated human protein, said protein comprising a tryptic digest peptide having the following partial sequence as determined by mass spectroscopy: PGLGF.

17. A preparation comprising an isolated human protein, said protein comprising a tryptic digest peptide having the following partial sequence as determined by mass spectroscopy: GPLGF.

18. A preparation comprising an isolated human protein, said protein comprising a tryptic digest peptide having the following partial sequence as determined by mass spectrometry: PGIGM.

19. A preparation comprising an isolated human protein, said protein comprising a tryptic digest peptide having the following partial sequence as determined by mass spectroscopy: GPIGM.

20. A preparation comprising an isolated human protein, said protein comprising a tryptic digest peptide having the following partial sequence as determined by mass spectroscopy: PGIGF.

21. A preparation comprising an isolated human protein, said protein comprising a tryptic digest peptide having the following partial sequence as determined by mass spectroscopy: GPIGF.

22. The preparation according to any one of claims 14, 15, 16, 17, 18, 19, 20 or 21, wherein the tryptic digest peptide has a mass of 1546.73 Da, and an N-terminal mass of 0 Da, and a C-terminal mass of 1076.63 Da, said masses having an error of measurement of 100 parts-per-million or less.

23. The preparation according to any one of claims 14, 15, 16, 17, 18, 19, 20 or 21, wherein the protein further comprising a tryptic digest peptide having the following partial sequence as determined by mass spectrometry: HQV.

24. The preparation according to any one of claims 14, 15, 16, 17, 18, 19, 20 or 21, wherein the protein further comprising a tryptic digest peptide having the following partial sequence as determined by mass spectrometry: HQV, wherein the tryptic digest peptide has a mass of 1096.56 Da, and an N-terminal mass of 0 Da, and a C-terminal mass of 733.50 Da, said masses having an error of measurement of 100 parts-per-million or less.

25. A preparation comprising an isolated human protein, said protein comprising a tryptic digest peptide having the following partial sequence as determined by mass spectroscopy: HQV.

26. The preparation according to claim 25 wherein the tryptic digest peptide has a mass of 1096.56 Da, and an N-terminal mass of 0 Da, and a C-terminal mass of 733.50 Da, said masses having an error of measurement of 100 parts-per-million or less.

27. The preparation according to any one of claims 14, 15, 16, 17, 18, 19, 20, 21, 25 or 26, wherein the protein has an isoelectric point (pI) of about 6.80 and an apparent molecular weight (MW) of about 18,741.

28. An isolated nucleic acid molecule that hybridizes to a nucleotide sequence encoding API-111, a nucleotide sequence encoding API-112, or their complements.

29. An isolated nucleic acid molecule that hybridizes to a nucleotide sequence encoding at least 10 consecutive amino acids of API-111 a nucleotide sequence encoding at least 10 consecutive amino acids of API-112, or their complements.

30. A vector comprising the nucleic acid molecule of claim 28 or 29.

31. A host cell comprising the vector of claim 28.

32. A host cell genetically engineered to express the nucleic acid molecule of claim 28 or 29.

33. An isolated nucleic acid molecule that hybridizes under highly stringent conditions or moderately stringent conditions to the following nucleic acid sequence: CCNGGNYTNGGNATG.

34. An isolated nucleic acid molecule that hybridizes under highly stringent conditions or moderately stringent conditions to the following nucleic acid sequence: GGNCCNYTNGGNATG.

35. An isolated nucleic acid molecule that hybridizes under highly stringent conditions or moderately stringent conditions to the following nucleic acid sequence: CCNGGNYTNGGNTTY.

36. An isolated nucleic acid molecule that hybridizes under highly stringent conditions or moderately stringent conditions to the following nucleic acid sequence: GGNCCNYTNGGNTTY.

37. An isolated nucleic acid molecule that hybridizes under highly stringent conditions or moderately stringent conditions to the following nucleic acid sequence: CCNGGNATHGGNATG.

38. An isolated nucleic acid molecule that hybridizes under highly stringent conditions or moderately stringent conditions to the following nucleic acid sequence: CCNGGNATHGGNTTY.

39. An isolated nucleic acid molecule that hybridizes under highly stringent conditions or moderately stringent conditions to the following nucleic acid sequence: GGNCCNATHGGNATG.

40. An isolated nucleic acid molecule that hybridizes under highly stringent conditions or moderately stringent conditions to the following nucleic acid sequence: GGNCCNATHGGNTTY.

41. The isolated nucleic acid molecule according to any one of claims 33, 34, 35, 36, 37, 38, 39, or 40, wherein the nucleic acid also hybridizes under highly stringent conditions or moderately stringent conditions to the following nucleic acid sequence: CAYCARGTN.

42. An isolated nucleic acid molecule that hybridizes under highly stringent conditions or moderately stringent conditions to the following nucleic acid sequence: CCCGGCCTGGGCATG.

43. An isolated nucleic acid molecule that hybridizes under highly stringent conditions or moderately stringent conditions to the following nucleic acid sequence: GGCCCCCTGGGCATG.

44. An isolated nucleic acid molecule that hybridizes under highly stringent conditions or moderately stringent conditions to the following nucleic acid sequence: CCCGGCCTGGGCTTC.

45. An isolated nucleic acid molecule that hybridizes under highly stringent conditions or moderately stringent conditions to the following nucleic acid sequence: GGCCCCCTGGGCTTC.

46. An isolated nucleic acid molecule that hybridizes under highly stringent conditions or moderately stringent conditions to the following nucleic acid sequence: CCCGGCATCGGCATG.

47. An isolated nucleic acid molecule that hybridizes under highly stringent conditions or moderately stringent conditions to the following nucleic acid sequence: CCCGGCATCGGCTTC.

48. An isolated nucleic acid molecule that hybridizes under highly stringent conditions or moderately stringent conditions to the following nucleic acid sequence: GGCCCCATCGGCATG.

49. An isolated nucleic acid molecule that hybridizes under highly stringent conditions or moderately stringent conditions to the following nucleic acid sequence: GGCCCCATCGGCTTC.

50. The isolated nucleic acid molecule according to any one of claims 42, 43, 44, 45, 46, 47, 48 or 49, wherein the nucleic acid also hybridizes under highly stringent conditions or moderately stringent conditions to the following nucleic acid sequence: CACCAGGTG.

51. A method for screening, diagnosis, or prognosis of Alzheimer's disease in a subject, the method comprising detecting, in a biological sample, a neural cell adhesion molecule (NCAM) that is an Alzheimer's Disease-Associated Protein Isoform (API), wherein a decreased level of said NCAM that is an API, relative to a control sample or a reference range, indicates the presence or degree of Alzheimer's disease or a subject at risk of developing Alzheimer's disease.

52. The method according to claim 51, wherein the NCAM that is an API is selected from the group consisting of: API-194 or an isoform or derivative thereof and API-214 or an isoform or derivative thereof.

53. The method of claim 51, wherein said detecting step comprises determining the presence of said NCAM that is an API by mass spectrometry.

54. The method of claim 51, wherein the step of detecting comprises:
(a) contacting the sample with a capture reagent that is specific for a NCAM that is an API; and
(b) detecting whether binding has occurred between said capture reagent and said NCAM that is an API in the sample.

55. The method of claim 54, wherein step (b) comprises detecting said captured NCAM that is an API using a directly or indirectly labeled detection reagent.

56. The method of claim 54, wherein the capture reagent is an antibody.

57. The method of claim 51, wherein said NCAM that is an API is quantitatively detected.

58. The method of claim 57, wherein the quantitatively detected NCAM that is an API is compared to a previously determined reference range or control.

59. An antibody capable of immunospecific binding to a NCAM that is an API.

60. The antibody of claim 59, wherein the antibody is capable of immunospecific binding to a NCAM that is an API selected from the group consisting of: API-194 or an isoform or derivative or fragment thereof and API-214 or an isoform or derivative or fragment thereof.

61. The method of claim 51, wherein the step of detecting comprises:
(a) contacting at least one oligonucleotide probe comprising 10 or more consecutive nucleotides complementary to a nucleotide sequence encoding a NCAM that is an API with RNA obtained from a biological sample from the subject or with cDNA copied from the RNA wherein said contacting occurs under conditions that permit hybridization of the probe to the nucleotide sequence if present;
(b) detecting hybridization, if any, between the probe and the nucleotide sequence; and
(c) comparing the hybridization, if any, detected in step (b) with the hybridization detected in a control sample, or with a previously determined reference range.

62. The method according to claim 61, wherein said NCAM that is an API is selected from the group consisting of: API-194 or an isoform or derivative thereof and API-214 or an isoform or derivative thereof.

63. The method as claimed in claim 61, wherein step (a) includes the step of hybridizing the nucleotide sequence to a DNA array, wherein one or more members of the array are the probes complementary to a plurality of nucleotide sequences encoding NCAMs that are APIs.

64. A kit comprising at least one reagent for detecting one or more NCAMs that are APIs according to the method of claim 1.

65. The kit of claim 64, further comprising instructions for use.

66. The kit of claim 64, wherein at least one reagent is an antibody capable of immunospecific binding to a NCAM that is an API or an isoform or derivative or fragment thereof.

67. The kit of claim 64, wherein at least one reagent is an nucleic acid encoding a NCAM that is an API or an isoform or derivative or fragment thereof.

68. A method of screening for agents that interact with a NCAM that is an API, said method comprising:
(a) contacting a NCAM that is an API or an isoform or derivative or fragment thereof or a fusion protein comprising a NCAM that is an API or an isoform or derivative or fragment thereof with a candidate agent; and
(b) determining whether or not the candidate agent interacts with said NCAM that is an API or an isoform or derivative or fragment thereof or said fusion protein comprising a NCAM that is an API or an isoform or derivative or fragment thereof.

69. The method according to claim 68, wherein the NCAM that is an API is selected from the group consisting of: API-1 94 and API-214.

70. A method of screening for or identifying agents that modulate the expression or activity of a NCAM that is an API, comprising:
(a) contacting a first cell expressing a NCAM that is an API or an isoform or derivative or fragment thereof or a fusion protein comprising a NCAM that is an API or an isoform or derivative or fragment thereof with a candidate agent;
(b) contacting a second cell expressing said NCAM that is an API or an isoform or derivative or fragment thereof or said fusion protein comprising a NCAM that is an API or an isoform or derivative or fragment thereof with a control agent; and
(c) comparing the levels of said NCAM that is an API or an isoform or derivative or fragment thereof or said fusion protein comprising a NCAM that is an API or an isoform or derivative or fragment thereof in the first and second populations of cells, or comparing the level of induction of a downstream effector of said NCAM that is an API or an isoform or derivative or fragment thereof or said fusion protein comprising a NCAM that is an API or an isoform or derivative or fragment thereof in the first and second cells.


SEQUENCE LISTING (492 sequences)

SEQ ID NO: 1 - protein, 8 aa, homo sapien
EDYICYAR

SEQ ID NO: 2 - protein, 11 aa, homo sapien
GKPPPSFSWTR

SEQ ID NO: 3 - protein, 9 aa, homo sapien
QPEYAVVQR

SEQ ID NO: 4 - protein, 12 aa, homo sapien
IIMLFTDGGEER

SEQ ID NO: 5 - protein, 10 aa, homo sapien
FVVTDGGITR

SEQ ID NO: 6 - protein, 10 aa, homo sapien
SGELEQEEER

SEQ ID NO: 7 - protein, 15 aa, homo sapien
EEEEEMAVVPQGLFR

SEQ ID NO: 8 - protein, 11 aa, homo sapien
LVNIYDSMPLR

SEQ ID NO: 9 - protein, 11 aa, homo sapien
VIVVWNNIGEK

SEQ ID NO: 10 - protein, 7 aa, homo sapien
YLELFQR

SEQ ID NO: 11 - protein, 11 aa, homo sapien
DCSGVSLHLTR

SEQ ID NO: 12 - protein, 9 aa, homo sapien
TEAYLEAIR

SEQ ID NO: 13 - protein, 11 aa, homo sapien
DGNPFYFTDHR

SEQ ID NO: 14 - protein, 13 aa, homo sapien
AETYEGVYQCTAR

SEQ ID NO: 15 - protein, 6 aa, homo sapien
FWDYLR

SEQ ID NO: 16 - protein, 15 aa, homo sapien
GEVQAMLGQSTEELR

SEQ ID NO: 17 - protein, 15 aa, homo sapien
KVEQAVETEPEPELR

SEQ ID NO: 18 - protein, 15 aa, homo sapien
SELEEQLTPVAEETR

SEQ ID NO: 19 - protein, 11 aa, homo sapien
VNSDGGLVALR

SEQ ID NO: 20 - protein, 12 aa, homo sapien
HYDGSYSTFGER

SEQ ID NO: 21 - protein, 11 aa, homo sapien
VGFYESDVMGR

SEQ ID NO: 22 - protein, 11 aa, homo sapien
LPPNVVEESAR

SEQ ID NO: 23 - protein, 10 aa, homo sapien
ADLSGITGAR

SEQ ID NO: 24 - protein, 9 aa, homo sapien
EIGELYLPK

SEQ ID NO: 25 - protein, 12 aa, homo sapien
FEDGVLDPDYPR

SEQ ID NO: 26 - protein, 11 aa, homo sapien
ELDESLQVAER

SEQ ID NO: 27 - protein, 10 aa, homo sapien
TEVQLEHLSR

SEQ ID NO: 28 - protein, 9 aa, homo sapien
EGPVLILGR

SEQ ID NO: 29 - protein, 9 aa, homo sapien
EPGEFALLR

SEQ ID NO: 30 - protein, 16 aa, homo sapien
TALASGGVLDASGDYR

SEQ ID NO: 31 - protein, 9 aa, homo sapien
YEAAVPDPR

SEQ ID NO: 32 - protein, 10 aa, homo sapien
VAMHLVCPSR

SEQ ID NO: 33 - protein, 14 aa, homo sapien
IVIGMDVAASEFYR

SEQ ID NO: 34 - protein, 10 aa, homo sapien
LGAEVYHTLK

SEQ ID NO: 35 - protein, 12 aa, homo sapien
VEQATQAIPMER

SEQ ID NO: 36 - protein, 10 aa, homo sapien
LSPYVNYSFR

SEQ ID NO: 37 - protein, 13 aa, homo sapien
AETYEGVYQCTAR

SEQ ID NO: 38 - protein, 11 aa, homo sapien
GKPPPSFSWTR

SEQ ID NO: 39 - protein, 14 aa, homo sapien
IDGDTIIFSNVQER

SEQ ID NO: 40 - protein, 13 aa, homo sapien
EGLDLQVLEDSGR

SEQ ID NO: 41 - protein, 9 aa, homo sapien
LICSELNGR

SEQ ID NO: 42 - protein, 10 aa, homo sapien
RTMRDQDTGK

SEQ ID NO: 43 - protein, 9 aa, homo sapien
YIFHNFMER

SEQ ID NO: 44 - protein, 16 aa, homo sapien
SPEQQETVLDGNLIIR

SEQ ID NO: 45 - protein, 13 aa, homo sapien
NGIDIYSLTVDSR

SEQ ID NO: 46 - protein, 8 aa, homo sapien
ILDDLSPR

SEQ ID NO: 47 - protein, 9 aa, homo sapien
IPTTFENGR

SEQ ID NO: 48 - protein, 13 aa, homo sapien
EDEEEEEGENYQK

SEQ ID NO: 49 - protein, 16 aa, homo sapien
GEAGAPGEEDIQGPTK

SEQ ID NO: 50 - protein, 16 aa, homo sapien
HLEEPGETQNAFLNER

SEQ ID NO: 51 - protein, 9 aa, homo sapien
EGPVLILGR

SEQ ID NO: 52 - protein, 9 aa, homo sapien
IVQFSPSGK

SEQ ID NO: 53 - protein, 8 aa, homo sapien
NNLVIFHR

SEQ ID NO: 54 - protein, 12 aa, homo sapien
ASSIIDELFQDR

SEQ ID NO: 55 - protein, 16 aa, homo sapien
TMLLQPAGSLGSYSYR

SEQ ID NO: 56 - protein, 16 aa, homo sapien
APEAQVSVQPNFQQDK

SEQ ID NO: 57 - protein, 10 aa, homo sapien
WLQGSQELPR

SEQ ID NO: 58 - protein, 10 aa, homo sapien
LSPYVNYSFR

SEQ ID NO: 59 - protein, 13 aa, homo sapien
AETYEGVYQCTAR

SEQ ID NO: 60 - protein, 11 aa, homo sapien
GKPPPSFSWTR

SEQ ID NO: 61 - protein, 14 aa, homo sapien
IDGDTIIFSNVQER

SEQ ID NO: 62 - protein, 13 aa, homo sapien
NALGAIHHTISVR

SEQ ID NO: 63 - protein, 9 aa, homo sapien
IALVITDGR

SEQ ID NO: 64 - protein, 12 aa, homo sapien
ALYLQYTDETFR

SEQ ID NO: 65 - protein, 12 aa, homo sapien
QSEDSTFYLGER

SEQ ID NO: 66 - protein, 13 aa, homo sapien
GAYPLSIEPIGVR

SEQ ID NO: 67 - protein, 27 aa, homo sapien
LVGGPMDASVEEEGVRALDFAVGEYNK

SEQ ID NO: 68 - protein, 14 aa, homo sapien
LAAAVSNFGYDLYR

SEQ ID NO: 69 - protein, 12 aa, homo sapien
TSLEDFYLDEER

SEQ ID NO: 70 - protein, 9 aa, homo sapien
YIETDPANR

SEQ ID NO: 71 - protein, 13 aa, homo sapien
AGALNSNDAFVLK

SEQ ID NO: 72 - protein, 11 aa, homo sapien
HVVPNEVVVQR

SEQ ID NO: 73 - protein, 9 aa, homo sapien
EPGEFALLR

SEQ ID NO: 74 - protein, 16 aa, homo sapien
TALASGGVLDASGDYR

SEQ ID NO: 75 - protein, 10 aa, homo sapien
VAMHLVCPSR

SEQ ID NO: 76 - protein, 10 aa, homo sapien
TGLEAISNHK

SEQ ID NO: 77 - protein, 9 aa, homo sapien
FFEECDPNK

SEQ ID NO: 78 - protein, 10 aa, homo sapien
QQTEWQSGQR

SEQ ID NO: 79 - protein, 15 aa, homo sapien
SELEEQLTPVAEETR

SEQ ID NO: 80 - protein, 13 aa, homo sapien
DVIATDKEDVAFK

SEQ ID NO: 81 - protein, 8 aa, homo sapien
ENFSCLTR

SEQ ID NO: 82 - protein, 12 aa, homo sapien
FVEGLPINDFSR

SEQ ID NO: 83 - protein, 9 aa, homo sapien
EVGVYEALK

SEQ ID NO: 84 - protein, 12 aa, homo sapien
LSELIQPLPLER

SEQ ID NO: 85 - protein, 9 aa, homo sapien
LVHGGPCDK

SEQ ID NO: 86 - protein, 11 aa, homo sapien
EKPGVYTNVCR

SEQ ID NO: 87 - protein, 7 aa, homo sapien
YTNWIQK

SEQ ID NO: 88 - protein, 10 aa, homo sapien
CSVFYGAPSK

SEQ ID NO: 89 - protein, 11 aa, homo sapien
LVNIYDSMPLR

SEQ ID NO: 90 - protein, 7 aa, homo sapien
YLELFQR

SEQ ID NO: 91 - protein, 12 aa, homo sapien
ITWSNPPAQGAR

SEQ ID NO: 92 - protein, 14 aa, homo sapien
VGGVQSLGGTGALR

SEQ ID NO: 93 - protein, 8 aa, homo sapien
IGADFLAR

SEQ ID NO: 94 - protein, 8 aa, homo sapien
NFGLYNER

SEQ ID NO: 95 - protein, 9 aa, homo sapien
HIYLLPSGR

SEQ ID NO: 96 - protein, 12 aa, homo sapien
LEGEACGVYTPR

SEQ ID NO: 97 - protein, 11 aa, homo sapien
LIVHNGYCDGR

SEQ ID NO: 98 - protein, 9 aa, homo sapien
LGPLVEQGR

SEQ ID NO: 99 - protein, 9 aa, homo sapien
LEEQAQQIR

SEQ ID NO: 100 - protein, 16 aa, homo sapien
LVGGPMDASVEEEGVR

SEQ ID NO: 101 - protein, 10 aa, homo sapien
EELLPAQDIK

SEQ ID NO: 102 - protein, 11 aa, homo sapien
GCPTEEGCGER

SEQ ID NO: 103 - protein, 11 aa, homo sapien
AASGTQNNVLR

SEQ ID NO: 104 - protein, 17 aa, homo sapien
ALYYDLISSPDIHGTYK

SEQ ID NO: 105 - protein, 11 aa, homo sapien
ELLDTVTAPQK

SEQ ID NO: 106 - protein, 14 aa, homo sapien
LAAAVSNFGYDLYR

SEQ ID NO: 107 - protein, 12 aa, homo sapien
TSLEDFYLDEER

SEQ ID NO: 108 - protein, 10 aa, homo sapien
THPHFVIPYR

SEQ ID NO: 109 - protein, 13 aa, homo sapien
QSLEASLAETEGR

SEQ ID NO: 110 - protein, 8 aa, homo sapien
YENEVALR

SEQ ID NO: 111 - protein, 10 aa, homo sapien
YEELQQTAGR

SEQ ID NO: 112 - protein, 9 aa, homo sapien
EPGEFALLR

SEQ ID NO: 113 - protein, 16 aa, homo sapien
TALASGGVLDASGDYR

SEQ ID NO: 114 - protein, 9 aa, homo sapien
YEAAVPDPR

SEQ ID NO: 115 - protein, 10 aa, homo sapien
VAMHLVCPSR

SEQ ID NO: 116 - protein, 10 aa, homo sapien
YLELESSGHR

SEQ ID NO: 117 - protein, 9 aa, homo sapien
AFLFQESPR

SEQ ID NO: 118 - protein, 13 aa, homo sapien
GLVSWGNIPCGSK

SEQ ID NO: 119 - protein, 11 aa, homo sapien
EKPGVYTNVCR

SEQ ID NO: 120 - protein, 29 aa, homo sapien
EKPGVYTNVCRDSCQGDSGGPLVCGDHLR

SEQ ID NO: 121 - protein, 16 aa, homo sapien
TMLLQPAGSLGSYSYR

SEQ ID NO: 122 - protein, 13 aa, homo sapien
NVPLPVIAELPPK

SEQ ID NO: 123 - protein, 8 aa, homo sapien
CFEPQLLR

SEQ ID NO: 124 - protein, 8 aa, homo sapien
EQPPSLTR

SEQ ID NO: 125 - protein, 11 aa, homo sapien
YWNDCEPPDSR

SEQ ID NO: 126 - protein, 14 aa, homo sapien
DSPVLIDFFEDTER

SEQ ID NO: 127 - protein, 14 aa, homo sapien
GGEGTGYFVDFSVR

SEQ ID NO: 128 - protein, 10 aa, homo sapien
VYLFDFPEGK

SEQ ID NO: 129 - protein, 9 aa, homo sapien
CISIYSSER

SEQ ID NO: 130 - protein, 12 aa, homo sapien
ASSIIDELFQDR

SEQ ID NO: 131 - protein, 10 aa, homo sapien
SADTLWDIQK

SEQ ID NO: 132 - protein, 10 aa, homo sapien
LKDDEVAQLK

SEQ ID NO: 133 - protein, 16 aa, homo sapien
LIAPVAEEEATVPNNK

SEQ ID NO: 134 - protein, 9 aa, homo sapien
EGPVLILGR

SEQ ID NO: 135 - protein, 8 aa, homo sapien
NNLVIFHR

SEQ ID NO: 136 - protein, 11 aa, homo sapien
LPPNVVEESAR

SEQ ID NO: 137 - protein, 16 aa, homo sapien
LVESGGGLVQPGGSLR

SEQ ID NO: 138 - protein, 15 aa, homo sapien
GEASVCVEDWESGDR

SEQ ID NO: 139 - protein, 14 aa, homo sapien
VSSQNIQDFPSVLR

SEQ ID NO: 140 - protein, 11 aa, homo sapien
LLEACTFHSAK

SEQ ID NO: 141 - protein, 11 aa, homo sapien
HSTVLENLPDK

SEQ ID NO: 142 - protein, 8 aa, homo sapien
DQYELLCR

SEQ ID NO: 143 - protein, 13 aa, homo sapien
QMDFELLCQNGAR

SEQ ID NO: 144 - protein, 15 aa, homo sapien
IECVSAENTEDCIAK

SEQ ID NO: 145 - protein, 13 aa, homo sapien
SPDFQLFSSSHGK

SEQ ID NO: 146 - protein, 12 aa, homo sapien
GSNFQWNQLQGK

SEQ ID NO: 147 - protein, 12 aa, homo sapien
CGLVPVLAENYK

SEQ ID NO: 148 - protein, 11 aa, homo sapien
WCTISNQEANK

SEQ ID NO: 149 - protein, 15 aa, homo sapien
FDQFFGEGCAPGSQR

SEQ ID NO: 150 - protein, 13 aa, homo sapien
EPVDNAENCHLAR

SEQ ID NO: 151 - protein, 11 aa, homo sapien
WCAIGHEETQK

SEQ ID NO: 152 - protein, 11 aa, homo sapien
HSTVLENLPDK

SEQ ID NO: 153 - protein, 14 aa, homo sapien
DNPQTHYYAVAVVK

SEQ ID NO: 154 - protein, 8 aa, homo sapien
DQYELLCR

SEQ ID NO: 155 - protein, 13 aa, homo sapien
QMDFELLCQNGAR

SEQ ID NO: 156 - protein, 10 aa, homo sapien
VTCVAEELLK

SEQ ID NO: 157 - protein, 11 aa, homo sapien
WCTISNQEANK

SEQ ID NO: 158 - protein, 13 aa, homo sapien
EPVDNAENCHLAR

SEQ ID NO: 159 - protein, 15 aa, homo sapien
FDQFFGEGCAPGSQR

SEQ ID NO: 160 - protein, 11 aa, homo sapien
HSTVLENLPDK

SEQ ID NO: 161 - protein, 14 aa, homo sapien
IPIEDGSGEVVLSR

SEQ ID NO: 162 - protein, 8 aa, homo sapien
DQYELLCR

SEQ ID NO: 163 - protein, 15 aa, homo sapien
FDQFFGEGCAPGSQR

SEQ ID NO: 164 - protein, 13 aa, homo sapien
SPDFQLFSSSHGK

SEQ ID NO: 165 - protein, 13 aa, homo sapien
EPVDNAENCHLAR

SEQ ID NO: 166 - protein, 12 aa, homo sapien
CGLVPVLAENYK

SEQ ID NO: 167 - protein, 11 aa, homo sapien
HSTVLENLPDK

SEQ ID NO: 168 - protein, 14 aa, homo sapien
ECCHGDLLECADDR

SEQ ID NO: 169 - protein, 12 aa, homo sapien
IYEATLEDCCAK

SEQ ID NO: 170 - protein, 13 aa, homo sapien
LGEYGFQNALIVR

SEQ ID NO: 171 - protein, 13 aa, homo sapien
DVFLGTFLYEYSR

SEQ ID NO: 172 - protein, 9 aa, homo sapien
FQPLVDEPK

SEQ ID NO: 173 - protein, 9 aa, homo sapien
GYTQQLAFR

SEQ ID NO: 174 - protein, 14 aa, homo sapien
AGDFLEANYMNLQR

SEQ ID NO: 175 - protein, 11 aa, homo sapien
LPLEYSYGEYR

SEQ ID NO: 176 - protein, 7 aa, homo sapien
LFEELVR

SEQ ID NO: 177 - protein, 14 aa, homo sapien
DPVQEAWAEDVDLR

SEQ ID NO: 178 - protein, 8 aa, homo sapien
GIFPVLCK

SEQ ID NO: 179 - protein, 9 aa, homo sapien
GDYPLEAVR

SEQ ID NO: 180 - protein, 14 aa, homo sapien
LSCAEDYLSLVLNR

SEQ ID NO: 181 - protein, 13 aa, homo sapien
LGEYGFQNALIVR

SEQ ID NO: 182 - protein, 12 aa, homo sapien
YICENQDTISTK

SEQ ID NO: 183 - protein, 9 aa, homo sapien
CCTESLVNR

SEQ ID NO: 184 - protein, 13 aa, homo sapien
DVFLGTFLYEYSR

SEQ ID NO: 185 - protein, 11 aa, homo sapien
HPDYSVSLLLR

SEQ ID NO: 186 - protein, 11 aa, homo sapien
INHGILYDEEK

SEQ ID NO: 187 - protein, 11 aa, homo sapien
EIMENYNIALR

SEQ ID NO: 188 - protein, 13 aa, homo sapien
ITCTEEGWSPTPK

SEQ ID NO: 189 - protein, 11 aa, homo sapien
YVMLPVADQEK

SEQ ID NO: 190 - protein, 13 aa, homo sapien
GSPAINVAVHVFR

SEQ ID NO: 191 - protein, 13 aa, homo sapien
ITVVDALHEIPVK

SEQ ID NO: 192 - protein, 8 aa, homo sapien
DNLAIQTR

SEQ ID NO: 193 - protein, 14 aa, homo sapien
KLVVENVDVLTQMR

SEQ ID NO: 194 - protein, 11 aa, homo sapien
GYCAPGMECVK

SEQ ID NO: 195 - protein, 14 aa, homo sapien
GTCEQGPSIVTPPK

SEQ ID NO: 196 - protein, 17 aa, homo sapien
AGAAAGGPGVSGVCVCK

SEQ ID NO: 197 - protein, 15 aa, homo sapien
KVEQAVETEPEPELR

SEQ ID NO: 198 - protein, 15 aa, homo sapien
SELEEQLTPVAEETR

SEQ ID NO: 199 - protein, 13 aa, homo sapien
GTFATLSELHCDK

SEQ ID NO: 200 - protein, 12 aa, homo sapien
VVAGVANALAHK

SEQ ID NO: 201 - protein, 10 aa, homo sapien
LLVVYPWTQR

SEQ ID NO: 202 - protein, 10 aa, homo sapien
VEEVKPLEGR

SEQ ID NO: 203 - protein, 12 aa, homo sapien
GPPGPPGGVVVR

SEQ ID NO: 204 - protein, 9 aa, homo sapien
VEVLAGDLR

SEQ ID NO: 205 - protein, 7 aa, homo sapien
FTFEYSR

SEQ ID NO: 206 - protein, 11 aa, homo sapien
FTDSENVCQER

SEQ ID NO: 207 - protein, 9 aa, homo sapien
VIALINDQR

SEQ ID NO: 208 - protein, 13 aa, homo sapien
TATSEYQTFFNPR

SEQ ID NO: 209 - protein, 10 aa, homo sapien
ELLESYIDGR

SEQ ID NO: 210 - protein, 13 aa, homo sapien
QEDDLANINQWVK

SEQ ID NO: 211 - protein, 11 aa, homo sapien
LCQDLGPGAFR

SEQ ID NO: 212 - protein, 13 aa, homo sapien
DVVLTTTFVDDIK

SEQ ID NO: 213 - protein, 12 aa, homo sapien
AIEDYINEFSVR

SEQ ID NO: 214 - protein, 10 aa, homo sapien
WLQGSQELPR

SEQ ID NO: 215 - protein, 16 aa, homo sapien
LVGGPMDASVEEEGVR

SEQ ID NO: 216 - protein, 11 aa, homo sapien
ALDFAVGEYNK

SEQ ID NO: 217 - protein, 10 aa, homo sapien
DQDGEILLPR

SEQ ID NO: 218 - protein, 12 aa, homo sapien
TSLEDFYLDEER

SEQ ID NO: 219 - protein, 9 aa, homo sapien
EPGEFALLR

SEQ ID NO: 220 - protein, 16 aa, homo sapien
TALASGGVLDASGDYR

SEQ ID NO: 221 - protein, 7 aa, homo sapien
YYTVFDR

SEQ ID NO: 222 - protein, 8 aa, homo sapien
QVFGEATK

SEQ ID NO: 223 - protein, 9 aa, homo sapien
IPTTFENGR

SEQ ID NO: 224 - protein, 14 aa, homo sapien
CPNPPVQENFDVNK

SEQ ID NO: 225 - protein, 11 aa, homo sapien
NILTSNNIDVK

SEQ ID NO: 226 - protein, 13 aa, homo sapien
NPNLPPETVDSLK

SEQ ID NO: 227 - protein, 16 aa, homo sapien
GEAGAPGEEDIQGPTK

SEQ ID NO: 228 - protein, 11 aa, homo sapien
ELDESLQVAER

SEQ ID NO: 229 - protein, 8 aa, homo sapien
FMETVAEK

SEQ ID NO: 230 - protein, 16 aa, homo sapien
EILSVDCSTNNPSQAK

SEQ ID NO: 231 - protein, 10 aa, homo sapien
LGQYASPTAK

SEQ ID NO: 232 - protein, 13 aa, homo sapien
GSFEFPVGDAVSK

SEQ ID NO: 233 - protein, 13 aa, homo sapien
EELVYELNPLDHR

SEQ ID NO: 234 - protein, 11 aa, homo sapien
ELDESLQVAER

SEQ ID NO: 235 - protein, 13 aa, homo sapien
GSPAINVAVHVFR

SEQ ID NO: 236 - protein, 13 aa, homo sapien
AADDTWEPFASGK

SEQ ID NO: 237 - protein, 12 aa, homo sapien
SWFEPLVEDMQR

SEQ ID NO: 238 - protein, 11 aa, homo sapien
LGADMEDVCGR

SEQ ID NO: 239 - protein, 9 aa, homo sapien
LEEQAQQIR

SEQ ID NO: 240 - protein, 15 aa, homo sapien
SELEEQLTPVAEETR

SEQ ID NO: 241 - protein, 15 aa, homo sapien
AATVGSLAGQPLQER

SEQ ID NO: 242 - protein, 8 aa, homo sapien
GPCWCVDR

SEQ ID NO: 243 - protein, 15 aa, homo sapien
HLDSVLQQLQTEVYR

SEQ ID NO: 244 - protein, 10 aa, homo sapien
KPNLQVFLGK

SEQ ID NO: 245 - protein, 13 aa, homo sapien
GLVSWGNIPCGSK

SEQ ID NO: 246 - protein, 11 aa, homo sapien
EKPGVYTNVCR

SEQ ID NO: 247 - protein, 18 aa, homo sapien
DSCQGDSGGPLVCGDHLR

SEQ ID NO: 248 - protein, 16 aa, homo sapien
SNLDEDIIAEENIVSR

SEQ ID NO: 249 - protein, 7 aa, homo sapien
NEQVEIR

SEQ ID NO: 250 - protein, 14 aa, homo sapien
SVTEQGAELSNEER

SEQ ID NO: 251 - protein, 16 aa, homo sapien
APEAQVSVQPNFQQDK

SEQ ID NO: 252 - protein, 16 aa, homo sapien
TMLLQPAGSLGSYSYR

SEQ ID NO: 253 - protein, 17 aa, homo sapien
AQGFTEDTIVFLPQTDK

SEQ ID NO: 254 - protein, 16 aa, homo sapien
TMLLQPAGSLGSYSYR

SEQ ID NO: 255 - protein, 17 aa, homo sapien
AQGFTEDTIVFLPQTDK

SEQ ID NO: 256 - protein, 9 aa, homo sapien
LPFVINDGK

SEQ ID NO: 257 - protein, 16 aa, homo sapien
TMLLQPAGSLGSYSYR

SEQ ID NO: 258 - protein, 17 aa, homo sapien
AQGFTEDTIVFLPQTDK

SEQ ID NO: 259 - protein, 16 aa, homo sapien
APEAQVSVQPNFQQDK

SEQ ID NO: 260 - protein, 17 aa, homo sapien
TQGFTEDAIVFLPQTDK

SEQ ID NO: 261 - protein, 12 aa, homo sapien
HVGDLGNVTADK

SEQ ID NO: 262 - protein, 14 aa, homo sapien
GDGPVQGIINFEQK

SEQ ID NO: 263 - protein, 16 aa, homo sapien
LVGGPMDASVEEEGVR

SEQ ID NO: 264 - protein, 11 aa, homo sapien
ALDFAVGEYNK

SEQ ID NO: 265 - protein, 11 aa, homo sapien
ELLDTVTAPQK

SEQ ID NO: 266 - protein, 12 aa, homo sapien
TSLEDFYLDEER

SEQ ID NO: 267 - protein, 9 aa, homo sapien
IPTTFENGR

SEQ ID NO: 268 - protein, 9 aa, homo sapien
QPEYAVVQR

SEQ ID NO: 269 - protein, 8 aa, homo sapien
ELDVLQGR

SEQ ID NO: 270 - protein, 7 aa, homo sapien
NNYMYAR

SEQ ID NO: 271 - protein, 9 aa, homo sapien
GLQDEDGYR

SEQ ID NO: 272 - protein, 7 aa, homo sapien
FACYYPR

SEQ ID NO: 273 - protein, 12 aa, homo sapien
AVMDDFAAFVEK

SEQ ID NO: 274 - protein, 12 aa, homo sapien
YICENQDSISSK

SEQ ID NO: 275 - protein, 15 aa, homo sapien
SELEEQLTPVAEETR

SEQ ID NO: 276 - protein, 16 aa, homo sapien
LVGGPMDASVEEEGVR

SEQ ID NO: 277 - protein, 11 aa, homo sapien
ALDFAVGEYNK

SEQ ID NO: 278 - protein, 15 aa, homo sapien
NYCGLPGEYWLGNDK

SEQ ID NO: 279 - protein, 8 aa, homo sapien
IRPFFPQQ

SEQ ID NO: 280 - protein, 13 aa, homo sapien
LESDVSAQMEYCR

SEQ ID NO: 281 - protein, 11 aa, homo sapien
DNDGWLTSDPR

SEQ ID NO: 282 - protein, 13 aa, homo sapien
AGALNSNDAFVLK

SEQ ID NO: 283 - protein, 8 aa, homo sapien
TGAQELLR

SEQ ID NO: 284 - protein, 7 aa, homo sapien
MTLDDFR

SEQ ID NO: 285 - protein, 13 aa, homo sapien
VFLDCCNYITELR

SEQ ID NO: 286 - protein, 13 aa, homo sapien
QSLEASLAETEGR

SEQ ID NO: 287 - protein, 15 aa, homo sapien
KVEQAVETEPEPELR

SEQ ID NO: 288 - protein, 12 aa, homo sapien
TSLEDFYLDEER

SEQ ID NO: 289 - protein, 15 aa, homo sapien
GEVQAMLGQSTEELR

SEQ ID NO: 290 - protein, 15 aa, homo sapien
KVEQAVETEPEPELR

SEQ ID NO: 291 - protein, 15 aa, homo sapien
SELEEQLTPVAEETR

SEQ ID NO: 292 - protein, 11 aa, homo sapien
QELSEAEQATR

SEQ ID NO: 293 - protein, 12 aa, homo sapien
TIYTPGSTVLYR

SEQ ID NO: 294 - protein, 14 aa, homo sapien
IPIEDGSGEVVLSR

SEQ ID NO: 295 - protein, 9 aa, homo sapien
GLQDEDGYR

SEQ ID NO: 296 - protein, 9 aa, homo sapien
ITQVLHFTK

SEQ ID NO: 297 - protein, 7 aa, homo sapien
FACYYPR

SEQ ID NO: 298 - protein, 7 aa, homo sapien
IWDVVEK

SEQ ID NO: 299 - protein, 11 aa, homo sapien
QPVPGQQMTLK

SEQ ID NO: 300 - protein, 12 aa, homo sapien
EVVADSVWVDVK

SEQ ID NO: 301 - protein, 10 aa, homo sapien
DSCVGSLVVK

SEQ ID NO: 302 - protein, 10 aa, homo sapien
DFDFVPPVVR

SEQ ID NO: 303 - protein, 16 aa, homo sapien
SNLDEDIIAEENIVSR

SEQ ID NO: 304 - protein, 14 aa, homo sapien
IPIEDGSGEVVLSR

SEQ ID NO: 305 - protein, 8 aa, homo sapien
CLVNLIEK

SEQ ID NO: 306 - protein, 17 aa, homo sapien
FLCTGGVSPYADPNTCR

SEQ ID NO: 307 - protein, 10 aa, homo sapien
VGDTLNLNLR

SEQ ID NO: 308 - protein, 13 aa, homo sapien
VFLDCCNYITELR

SEQ ID NO: 309 - protein, 9 aa, homo sapien
FISLGEACK

SEQ ID NO: 310 - protein, 16 aa, homo sapien
LVGGPMDASVEEEGVR

SEQ ID NO: 311 - protein, 11 aa, homo sapien
ALDFAVGEYNK

SEQ ID NO: 312 - protein, 13 aa, homo sapien
AADDTWEPFASGK

SEQ ID NO: 313 - protein, 14 aa, homo sapien
LISWYDNEFGYSNR

SEQ ID NO: 314 - protein, 14 aa, homo sapien
VPTANVSVVDLTCR

SEQ ID NO: 315 - protein, 11 aa, homo sapien
ISYQSSSTEER

SEQ ID NO: 316 - protein, 11 aa, homo sapien
TQVNTQAEQLR

SEQ ID NO: 317 - protein, 10 aa, homo sapien
ALVQQMEQLR

SEQ ID NO: 318 - protein, 15 aa, homo sapien
VLSLAQEQVGGSPEK

SEQ ID NO: 319 - protein, 13 aa, homo sapien
AEMADQAAAWLTR

SEQ ID NO: 320 - protein, 9 aa, homo sapien
QGSFQGGFR

SEQ ID NO: 321 - protein, 10 aa, homo sapien
LVMGIPTFGR

SEQ ID NO: 322 - protein, 13 aa, homo sapien
EGDGSCFPDALDR

SEQ ID NO: 323 - protein, 13 aa, homo sapien
FSNTDYAVGYMLR

SEQ ID NO: 324 - protein, 14 aa, homo sapien
GNQWVGYDDQESVK

SEQ ID NO: 325 - protein, 8 aa, homo sapien
QHFTTLIK

SEQ ID NO: 326 - protein, 18 aa, homo sapien
DAIPEDLPPLTADFAEDK

SEQ ID NO: 327 - protein, 7 aa, homo sapien
YLYEIAR

SEQ ID NO: 328 - protein, 13 aa, homo sapien
VFLDCCNYITELR

SEQ ID NO: 329 - protein, 16 aa, homo sapien
SNLDEDIIAEENIVSR

SEQ ID NO: 330 - protein, 9 aa, homo sapien
GYTQQLAFR

SEQ ID NO: 331 - protein, 9 aa, homo sapien
IDQTVEELR

SEQ ID NO: 332 - protein, 11 aa, homo sapien
TQVNTQAEQLR

SEQ ID NO: 333 - protein, 10 aa, homo sapien
ALVQQMEQLR

SEQ ID NO: 334 - protein, 9 aa, homo sapien
LEPYADQLR

SEQ ID NO: 335 - protein, 13 aa, homo sapien
AADDTWEPFASGK

SEQ ID NO: 336 - protein, 15 aa, homo sapien
GECQAEGVLFFQGDR

SEQ ID NO: 337 - protein, 11 aa, homo sapien
YYCFQGNQFLR

SEQ ID NO: 338 - protein, 12 aa, homo sapien
TIYTPGSTVLYR

SEQ ID NO: 339 - protein, 15 aa, homo sapien
TVMVNIENPEGIPVK

SEQ ID NO: 340 - protein, 11 aa, homo sapien
ELDESLQVAER

SEQ ID NO: 341 - protein, 16 aa, homo sapien
EILSVDCSTNNPSQAK

SEQ ID NO: 342 - protein, 9 aa, homo sapien
LGPLVEQGR

SEQ ID NO: 343 - protein, 15 aa, homo sapien
AATVGSLAGQPLQER

SEQ ID NO: 344 - protein, 9 aa, homo sapien
DNCCILDER

SEQ ID NO: 345 - protein, 13 aa, homo sapien
YEASILTHDSSIR

SEQ ID NO: 346 - protein, 10 aa, homo sapien
TSTADYAMFK

SEQ ID NO: 347 - protein, 13 aa, homo sapien
VAQLEAQCQEPCK

SEQ ID NO: 348 - protein, 9 aa, homo sapien
VELEDWNGR

SEQ ID NO: 349 - protein, 12 aa, homo sapien
YLQEIYNSNNQK

SEQ ID NO: 350 - protein, 9 aa, homo sapien
RLDGSVDFK

SEQ ID NO: 351 - protein, 13 aa, homo sapien
GLIDEVNQDFTNR

SEQ ID NO: 352 - protein, 16 aa, homo sapien
ADSGEGDFLAEGGGVR

SEQ ID NO: 353 - protein, 13 aa, homo sapien
GLIDEVNQDFTNR

SEQ ID NO: 354 - protein, 15 aa, homo sapien
ESSSHHPGIAEFPSR

SEQ ID NO: 355 - protein, 11 aa, homo sapien
SGNENGEFYLR

SEQ ID NO: 356 - protein, 10 aa, homo sapien
DQDGEILLPR

SEQ ID NO: 357 - protein, 12 aa, homo sapien
DCQPGLCCAFQR

SEQ ID NO: 358 - protein, 10 aa, homo sapien
DQDGEILLPR

SEQ ID NO: 359 - protein, 13 aa, homo sapien
SLDFTELDVAAEK

SEQ ID NO: 360 - protein, 12 aa, homo sapien
ALQDQLVLVAAK

SEQ ID NO: 361 - protein, 12 aa, homo sapien
LNMGITDLQGLR

SEQ ID NO: 362 - protein, 10 aa, homo sapien
VGDTLNLNLR

SEQ ID NO: 363 - protein, 8 aa, homo sapien
KLCMAALK

SEQ ID NO: 364 - protein, 8 aa, homo sapien
ELPEHTVK

SEQ ID NO: 365 - protein, 10 aa, homo sapien
THLPEVFLSK

SEQ ID NO: 366 - protein, 11 aa, homo sapien
HLSLLTTLSNR

SEQ ID NO: 367 - protein, 8 aa, homo sapien
FEDCCQEK

SEQ ID NO: 368 - protein, 11 aa, homo sapien
LPEATPTELAK

SEQ ID NO: 369 - protein, 11 aa, homo sapien
VCSQYAAYGEK

SEQ ID NO: 370 - protein, 7 aa, homo sapien
YTFELSR

SEQ ID NO: 371 - protein, 8 aa, homo sapien
LCDNLSTK

SEQ ID NO: 372 - protein, 13 aa, homo sapien
SLDFTELDVAAEK

SEQ ID NO: 373 - protein, 12 aa, homo sapien
DPTFIPAPIQAK

SEQ ID NO: 374 - protein, 12 aa, homo sapien
LQSLFDSPDFSK

SEQ ID NO: 375 - protein, 14 aa, homo sapien
LAAAVSNFGYDLYR

SEQ ID NO: 376 - protein, 12 aa, homo sapien
TSLEDFYLDEER

SEQ ID NO: 377 - protein, 9 aa, homo sapien
EPGEFALLR

SEQ ID NO: 378 - protein, 16 aa, homo sapien
TALASGGVLDASGDYR

SEQ ID NO: 379 - protein, 10 aa, homo sapien
VAMHLVCPSR

SEQ ID NO: 380 - protein, 9 aa, homo sapien
DHAVDLIQK

SEQ ID NO: 381 - protein, 12 aa, homo sapien
TEQWSTLPPETK

SEQ ID NO: 382 - protein, 15 aa, homo sapien
VLSLAQEQVGGSPEK

SEQ ID NO: 383 - protein, 9 aa, homo sapien
QGSFQGGFR

SEQ ID NO: 384 - protein, 11 aa, homo sapien
ADGSYAAWLSR

SEQ ID NO: 385 - protein, 13 aa, homo sapien
AEMADQASAWLTR

SEQ ID NO: 386 - protein, 11 aa, homo sapien
TQVNTQAEQLR

SEQ ID NO: 387 - protein, 11 aa, homo sapien
ELDESLQVAER

SEQ ID NO: 388 - protein, 7 aa, homo sapien
KYNELLK

SEQ ID NO: 389 - protein, 13 aa, homo sapien
AQLGDLPWQVAIK

SEQ ID NO: 390 - protein, 10 aa, homo sapien
VFSLQWGEVK

SEQ ID NO: 391 - protein, 9 aa, homo sapien
LGPIEAIQK

SEQ ID NO: 392 - protein, 9 aa, homo sapien
LGPLVEQGR

SEQ ID NO: 393 - protein, 9 aa, homo sapien
LEEQAQQIR

SEQ ID NO: 394 - protein, 7 aa, homo sapien
KMEENEK

SEQ ID NO: 395 - protein, 11 aa, homo sapien
ELDESLQVAER

SEQ ID NO: 396 - protein, 9 aa, homo sapien
IDSLLENDR

SEQ ID NO: 397 - protein, 15 aa, homo sapien
EDALNETRESETKLK

SEQ ID NO: 398 - protein, 16 aa, homo sapien
EILSVDCSTNNPSQAK

SEQ ID NO: 399 - protein, 10 aa, homo sapien
TLLSNLEEAK

SEQ ID NO: 400 - protein, 15 aa, homo sapien
SELEEQLTPVAEETR

SEQ ID NO: 401 - protein, 15 aa, homo sapien
AATVGSLAGQPLQER

SEQ ID NO: 402 - protein, 9 aa, homo sapien
GLQDEDGYR

SEQ ID NO: 403 - protein, 7 aa, homo sapien
FACYYPR

SEQ ID NO: 404 - protein, 8 aa, homo sapien
LLEVPEGR

SEQ ID NO: 405 - protein, 11 aa, homo sapien
TNFDNDIALVR

SEQ ID NO: 406 - protein, 16 aa, homo sapien
SNLDEDIIAEENIVSR

SEQ ID NO: 407 - protein, 16 aa, homo sapien
VELLHNPAFCSLATTK

SEQ ID NO: 408 - protein, 12 aa, homo sapien
LSELIQPLPLER

SEQ ID NO: 409 - protein, 9 aa, homo sapien
LLIYWASTR

SEQ ID NO: 410 - protein, 16 aa, homo sapien
SGTASVVCLLNNFYPR

SEQ ID NO: 411 - protein, 14 aa, homo sapien
EVDSGNDIYGNPIK

SEQ ID NO: 412 - protein, 9 aa, homo sapien
SDGSCAWYR

SEQ ID NO: 413 - protein, 13 aa, homo sapien
AETYEGVYQCTAR

SEQ ID NO: 414 - protein, 11 aa, homo sapien
GKPPPSFSWTR

SEQ ID NO: 415 - protein, 14 aa, homo sapien
IDGDTIIFSNVQER

SEQ ID NO: 416 - protein, 12 aa, homo sapien
LNMGITDLQGLR

SEQ ID NO: 417 - protein, 10 aa, homo sapien
VGDTLNLNLR

SEQ ID NO: 418 - protein, 9 aa, homo sapien
EPGEFALLR

SEQ ID NO: 419 - protein, 16 aa, homo sapien
TALASGGVLDASGDYR

SEQ ID NO: 420 - protein, 9 aa, homo sapien
YEAAVPDPR

SEQ ID NO: 421 - protein, 9 aa, homo sapien
IDQTVEELR

SEQ ID NO: 422 - protein, 11 aa, homo sapien
TQVNTQAEQLR

SEQ ID NO: 423 - protein, 12 aa, homo sapien
SLAPYAQDTQEK

SEQ ID NO: 424 - protein, 10 aa, homo sapien
ALVQQMEQLR

SEQ ID NO: 425 - protein, 9 aa, homo sapien
LEPYADQLR

SEQ ID NO: 426 - protein, 11 aa, homo sapien
RVEPYGENFNK

SEQ ID NO: 427 - protein, 12 aa, homo sapien
TSLEDFYLDEER

SEQ ID NO: 428 - protein, 9 aa, homo sapien
AVFPSIVGR

SEQ ID NO: 429 - protein, 16 aa, homo sapien
SYELPDGQVITIGNER

SEQ ID NO: 430 - protein, 10 aa, homo sapien
AGFAGDDAPR

SEQ ID NO: 431 - protein, 10 aa, homo sapien
GYSFTTTAER

SEQ ID NO: 432 - protein, 13 aa, homo sapien
QEYDESGPSIVHR

SEQ ID NO: 433 - protein, 18 aa, homo sapien
VAPEEHPVLLTEAPLNPK

SEQ ID NO: 434 - protein, 13 aa, homo sapien
EELVYELNPLDHR

SEQ ID NO: 435 - protein, 14 aa, homo sapien
EPFLSCCQFAESLR

SEQ ID NO: 436 - protein, 11 aa, homo sapien
GLCVATPVQLR

SEQ ID NO: 437 - protein, 13 aa, homo sapien
EELVYELNPLDHR

SEQ ID NO: 438 - protein, 12 aa, homo sapien
ASSIIDELFQDR

SEQ ID NO: 439 - protein, 10 aa, homo sapien
TLLSNLEEAK

SEQ ID NO: 440 - protein, 24 aa, homo sapien
GEVQAMLGQSTEELRLEEQAQQIR

SEQ ID NO: 441 - protein, 15 aa, homo sapien
SELEEQLTPVAEETR

SEQ ID NO: 442 - protein, 12 aa, homo sapien
ALEESNYELEGK

SEQ ID NO: 443 - protein, 13 aa, homo sapien
GSFEFPVGDAVSK

SEQ ID NO: 444 - protein, 11 aa, homo sapien
GLCVATPVQLR

SEQ ID NO: 445 - protein, 13 aa, homo sapien
EELVYELNPLDHR

SEQ ID NO: 446 - protein, 14 aa, homo sapien
EPFLSCCQFAESLR

SEQ ID NO: 447 - protein, 16 aa, homo sapien
TMLLQPAGSLGSYSYR

SEQ ID NO: 448 - protein, 17 aa, homo sapien
AQGFTEDTIVFLPQTDK

SEQ ID NO: 449 - protein, 13 aa, homo sapien
GSPAINVAVHVFR

SEQ ID NO: 450 - protein, 13 aa, homo sapien
AADDTWEPFASGK

SEQ ID NO: 451 - protein, 11 aa, homo sapien
LIVHNGYCDGR

SEQ ID NO: 452 - protein, 8 aa, homo sapien
QEELCLAR

SEQ ID NO: 453 - protein, 10 aa, homo sapien
FSGTWYAMAK

SEQ ID NO: 454 - protein, 10 aa, homo sapien
CSVFYGAPSK

SEQ ID NO: 455 - protein, 9 aa, homo sapien
GLQDEDGYR

SEQ ID NO: 456 - protein, 13 aa, homo sapien
AADDTWEPFASGK

SEQ ID NO: 457 - protein, 9 aa, homo sapien
VGYVSGWGR

SEQ ID NO: 458 - protein, 11 aa, homo sapien
SGNENGEFYLR

SEQ ID NO: 459 - protein, 9 aa, homo sapien
ADQVCINLR

SEQ ID NO: 460 - protein, 11 aa, homo sapien
ELLDTVTAPQK

SEQ ID NO: 461 - protein, 14 aa, homo sapien
LAAAVSNFGYDLYR

SEQ ID NO: 462 - protein, 12 aa, homo sapien
TSLEDFYLDEER

SEQ ID NO: 463 - protein, 17 aa, homo sapien
ALYYDLISSPDIHGTYK

SEQ ID NO: 464 - protein, 3 aa, homo sapien
HQV

SEQ ID NO: 465 - DNA, 9 bp, Artificial Sequence
  misc_feature (0)...(0): primer
caccaggtg

SEQ ID NO: 466 - DNA, 9 bp, Artificial Sequence
  misc_feature (1)...(9): n = A,T,C or G
  misc_feature (1)...(90): primer
caycargtn

SEQ ID NO: 467 - protein, 5 aa, homo sapien
PGLGM

SEQ ID NO: 468 - DNA, 15 bp, Artificial Sequence
  misc_feature (1)...(15): primer
cccggcctgggcatg

SEQ ID NO: 469 - DNA, 15 bp, Artificial Sequence
  misc_feature (1)...(15): n = A,T,C or G
  misc_feature (1)...(15): primer
ccnggnytnggnatg

SEQ ID NO: 470 - protein, 5 aa, homo sapien
PGLGF

SEQ ID NO: 471 - DNA, 15 bp, Artificial Sequence
  misc_feature (1)...(15): primer
cccggcctgggcttc

SEQ ID NO: 472 - DNA, 15 bp, Artificial Sequence
  misc_feature (1)...(15): n = A,T,C or G
  misc_feature (1)...(15): primer
ccnggnytnggntty

SEQ ID NO: 473 - protein, 5 aa, homo sapien
PGIGM

SEQ ID NO: 474 - DNA, 15 bp, Artificial Sequence
  misc_feature (1)...(15): primer
cccggcatcggcatg

SEQ ID NO: 475 - DNA, 15 bp, Artificial Sequence
  misc_feature (1)...(15): n = A,T,C or G
  misc_feature (1)...(15): primer
ccnggnathggnatg

SEQ ID NO: 476 - protein, 5 aa, homo sapien
PGIGF

SEQ ID NO: 477 - DNA, 15 bp, Artificial Sequence
  primer
cccggcatcggcttc

SEQ ID NO: 478 - DNA, 15 bp, Artificial Sequence
  misc_feature (1)...(15): n = A,T,C or G
  misc_feature (1)...(15): primer
ccnggnathggntty

SEQ ID NO: 479 - protein, 5 aa, homo sapien
GPLGM

SEQ ID NO: 480 - DNA, 15 bp, Artificial Sequence
  misc_feature (1)...(15): primer
ggccccctgggcatg

SEQ ID NO: 481 - DNA, 15 bp, Artificial Sequence
  misc_feature (1)...(15): n = A,T,C or G
  misc_feature (1)...(15): primer
ggnccnytnggnatg

SEQ ID NO: 482 - protein, 5 aa, homo sapien
GPLGF

SEQ ID NO: 483 - DNA, 15 bp, Artificial Sequence
  misc_feature (1)...(15): primer
ggccccctgggcttc

SEQ ID NO: 484 - DNA, 15 bp, Artificial Sequence
  misc_feature (1)...(15): n = A,T,C or G
  misc_feature (1)...(15): primer
ggnccnytnggntty

SEQ ID NO: 485 - protein, 5 aa, homo sapien
GPIGM

SEQ ID NO: 486 - DNA, 15 bp, Artificial Sequence
  misc_feature (1)...(15): primer
ggccccatcggcatg

SEQ ID NO: 487 - DNA, 15 bp, Artificial Sequence
  misc_feature (1)...(15): n = A,T,C or G
  misc_feature (1)...(15): primer
ggnccnathggnatg

SEQ ID NO: 488 - protein, 5 aa, homo sapien
GPIGF

SEQ ID NO: 489 - DNA, 15 bp, Artificial Sequence
  misc_feature (1)...(15): primer
  misc_feature (1)...(15): n = A,T,C or G
ggnccnathggntty

SEQ ID NO: 490 - DNA, 15 bp, Artificial Sequence
  misc_feature (1)...(15): primer
  misc_feature (1)...(15): n = A,T,C or G
ggnccnathggntty

SEQ ID NO: 491 - DNA, 15 bp, Artificial Sequence
  misc_feature (1)...(15): n = A, T, C or G
  misc_feature (1)...(15): primer
ggccccatcggcatg

SEQ ID NO: 492 - DNA, 15 bp, Artificial Sequence
  misc_feature (1)...(15): n = A, T, C or G
  misc_feature (1)...(15): primer
ggccccatcggcttg